SEQUENCE LISTING (8 sequences)

SEQ ID NO: 1 - DNA, 20 bp, Artificial Sequence
  iNOS forward
caccttggagttcacccagt

SEQ ID NO: 2 - DNA, 20 bp, Artificial Sequence
  iNOS  reverse
accactcgtacttgggatgc

SEQ ID NO: 3 - DNA, 20 bp, Artificial Sequence
  COX2 forward
cccccacagtcaaagacact

SEQ ID NO: 4 - DNA, 20 bp, Artificial Sequence
  COX2 reverse
ctcatcaccccactcaggat

SEQ ID NO: 5 - DNA, 20 bp, Artificial Sequence
  TNFalpha forward
agaagttcccaaatggcctc

SEQ ID NO: 6 - DNA, 20 bp, Artificial Sequence
  TNFalpha reverse
gtctttgagatccatgccgt

SEQ ID NO: 7 - DNA, 20 bp, Artificial Sequence
  IL1 beta forward
tgtgaaatgccaccttttga

SEQ ID NO: 8 - DNA, 20 bp, Artificial Sequence
  IL1 beta reverse
tgagtgatactgcctgcctg

(19) United States
(12) Patent Application Publication
(10) Pub. No.: US 2012/0136057 A1
(43) Pub. Date: May 31, 2012

(54) HYDROXY FATTY ACID COMPOUNDS AND USES THEREOF FOR DISEASE TREATMENT AND DIAGNOSIS

(75) Inventors: Shawn Ritchie, Saskatoon (CA); Dayan Goodenowe, Saskatoon (CA); M. Amin Khan, Saskatoon (CA); Pearson W.K. Ahiahonu, Saskatoon (CA)

(73) Assignee: PHENOMENOME DISCOVERIES INC., Saskatoon, SK (CA)

(21) Appl. No.: 13/387,110

(22) PCT Filed: Jul. 29, 2010
(86) PCT No.: PCT/CA2010/001179

§ 371 Date: Jan. 25, 2012

Related U.S. Application Data
(60) Provisional application No. 61/229,566, filed on Jul. 29, 2009.

(51) Int. Cl. A61K 31/20; A61P 35/00; A61P 1/00; A61P 29/00; H01J 49/26; C07K 14/00; C12N 9/96; C07F 7/08; C07C 31/18; C07C 69/52; C07C 59/42; G01N 33/566

(52) U.S. Cl. 514/560; 554/219; 435/7.92; 530/350; 435/188; 556/431; 556/446; 568/852; 560/205; 250/282

(57) ABSTRACT

A compound of formula (I): wherein R represents a hydroxy substituted C₂₄-C₄₀ straight chain aliphatic group containing at least one double bond in the carbon chain; and at least one carbon in the chain is substituted with a hydroxy group. Such compounds are useful for detecting inflammation, inflammatory disorders and cancer in a subject, and can also be used in therapeutic applications including treatment and/or prevention of these conditions. Pharmaceutical compositions, combinations and supplements, as well as methods of treatment using the described compounds are therefore also described.

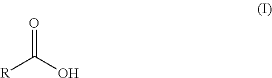

### FIELD OF INVENTION

The present invention relates to compounds useful in detection and treatment of diseases and physiological conditions. More specifically, the invention relates to hydroxy fatty acid compounds, compositions comprising same, and methods using these compounds for treating and detecting colorectal cancer, inflammation and inflammatory diseases.

### BACKGROUND OF THE INVENTION

Colorectal cancer (CRC) mortality remains one of the highest among all cancers, second to only lung cancer (Canadian Cancer Statistics, 2008). Despite the known benefits of early detection, screening programs based on colonoscopy and fecal occult blood testing have been plagued with challenges such as public acceptance, cost, limited resources, accuracy, and standardization. There is consensus in the field that the use of colonoscopy alone for CRC screening is not practical¹, and that a minimally-invasive serum-based test capable of accurately identifying subjects who are high risk for the development of CRC would result in a higher screening compliance than current approaches and better utilization of existing endoscopy resources¹⁻³. Although there have been multiple reports of altered transcript levels⁴⁻¹¹, aberrantly methylated gene products¹²⁻¹⁴ and proteomic patterns¹⁵⁻¹⁸ associated with biological samples from CRC patients, few if any have advanced into clinically useful tests. This may be due to a number of reasons including technical hurdles in assay design, challenges obtaining reproducible results, costs, and lengthy regulatory processes. Furthermore, most of the tests currently used or in development are based upon the detection of tumor-specific markers, and have poor sensitivity for identifying subjects who are either very early stage, or are predisposed to risk but show no clinical presentation of disease.

Although causal genetic alterations for CRC have been well characterized, the number of cases due to adenomatous polyposis coli (APC) and hereditary nonpolyposis colorectal cancer (HNPCC) are less than 5% of the total, with approximately 15% claimed to be attributable to inheritable family risk likely due to complex patterns of low penetrance mutations which have yet to be delineated¹⁹. The fact remains that approximately 80% of CRC cases are thought to arise sporadically, with diet and lifestyle as key risk factors^{20, 21}. In addition, an individual's microbiome is intricately linked to their gastrointestinal physiological status, and may itself be involved as a risk factor²². Given that metabolism is heavily influenced by both diet and lifestyle, and that the microbiome contributes its own metabolic processes, it is surprising that there has been little effort aimed at identifying metabolic markers as risk indicators of CRC. This may, in part, have been due to the lack of platform technologies and informatics approaches capable of comprehensively characterizing metabolites in a similar way that DNA microarrays or surface-enhanced laser desorption/ionization (SELDI) can characterize transcripts or proteins, respectively.

The need for accurate methods for detecting CRC therefore remains, particularly for methods to detect early stages of the disease.

### Mass Spectrometric-Based Systems for Metabolite Analysis

Recently there have been advances made in mass spectrometric-based systems which can identify large numbers of metabolic components within samples in a parallel manner²³⁻²⁵.

Fourier transform ion cyclotron resonance mass spectrometry (FTICR-MS) is based upon the principle that charged particles exhibit cyclotron motion in a magnetic field, where the spin frequency is proportional to the mass²⁶. FTICR-MS is known for its high resolving power and capability of detecting ions with mass accuracy below 1 part per million (ppm). Liquid sample extracts can be directly infused using electrospray ionization (ESI) and atmospheric pressure chemical ionization (APCI) without chromatographic separation²³, where ions with differing mass to charge (M/Z) ratios can be simultaneously resolved using a Fourier transformation. Using informatics approaches, spectral files from multiple samples can be accurately aligned and peak intensities across the samples compared²³. High resolution also enables the prediction of elemental composition of all ions detected in a sample, providing a solid foundation for metabolite classification and identification, as well as the ability to construct de novo metabolic networks^{23, 27}.

Nevertheless, without accurate serum markers available for detecting CRC in the early stages of disease, mass-spectrometry-based diagnostic systems have not been widely used in clinical testing.

### Role of Bioactive Lipids in Inflammation and Disease

Inflammation is a critical underlying component to many human diseases, including cancer. Understanding how inflammation arises and is controlled by the body, and how dietary and environmental factors impact inflammation is important for disease prevention and treatment.

The role of bioactive lipids in inflammation was reported in 1979 by Borgeat and Samuelsson, who showed that arachidonic acid gives rise to various pro-inflammatory mediators, the prostaglandins and leukotrienes, through the activity of cyclooxygenases and lipoxygenases (Borgeat P, Samuelsson B. Metabolism of arachidonic acid in polymorphonuclear leukocytes. Structural analysis of novel hydroxylated compounds. J. Biol. Chem., 1979, 254:7865-9). Since that time there has been a preponderance of data reported suggesting that polyunsaturated fatty acids (PUFAs) can also have beneficial health effects and can protect against a number of inflammation-associated disorders including cancer (Chapkin R S, Davidson L A, Ly L, et al. Immunomodulatory effects of (n-3) fatty acids: putative link to inflammation and colon cancer. J. Nutr. 2007, 137:200 S-204S; Chapkin R S, McMurray D N, Lupton J R. Colon cancer, fatty acids and anti-inflammatory compounds. Curr. Opin. Gastroenterol, 2007, 23:48-54; Chapkin R S, Seo J, McMurray D N, et al. Mechanisms by which docosahexaenoic acid and related fatty acids reduce colon cancer risk and inflammatory disorders of the intestine. Chem. Phys. Lipids 2008, 153:14-23). Of particular interest are the roles of n-3, as well as n-6 fatty acids in the resolution of inflammation.

The n-3 class of PUFAs are enriched in fish oils, and are defined by the position of the first double-bond from the methyl position of the acyl chain. The very long-chain docosahexaenoic acid (DHA; 22:6n-3) and eicosapentaenoic acid (EPA; 20:5n-3) are high abundance n-3 PUFAs in fish oils, while the shorter-chain linolenic acid (LNA; 18:3n-3) is abundant in seed oils such as flax and canola. Endogenous levels of n-3 fatty acids are heavily influenced by diet, although their synthesis in vivo is possible. The exact mechanisms of n-3 anti-inflammatory activity are poorly understood and diverse in nature. However, many of the pleiotropic effects can be attributed to modulation of cell membrane architecture and fluidity, inhibition of prostaglandin synthesis, regulation of NF-κB through PPARs and Toll-like receptors, alterations in protein targeting, and the conversion into various inflammation-resolving products (Chapkin R S, Davidson L A, Ly L, et al. Immunomodulatory effects of (n-3) fatty acids: putative link to inflammation and colon cancer. J. Nutr. 2007, 137:200 S-204S; Chapkin R S, McMurray D N, Lupton J R. Colon cancer, fatty acids and anti-inflammatory compounds. Curr. Opin. Gastroenterol. 2007, 23:48-54; Chapkin R S, Seo J, McMurray D N, et al. Mechanisms by which docosahexaenoic acid and related fatty acids reduce colon cancer risk and inflammatory disorders of the intestine. Chem. Phys. Lipids 2008, 153:14-23).

Acute inflammation is a short-term response to infection, injury or trauma, and is characterized by the release of pro-inflammatory mediators such as leukotrienes and prostaglandins derived from n-6 arachidonic acid, which in combination with other chemo-attractants results in the recruitment of leukocytes to the site of infection or injury. This initial wave of inflammation is soon thereafter accompanied by a wave of resolution, in which further PMN recruitment is checked through a platelet-leukocyte interaction that generates lipoxygenase-derived eicosanoids, also from arachidonic acid. The resulting lipoxins are highly potent and act at pictogram quantities. They can also be aspirin-triggered, giving aspirin a unique ability among non-steroidal anti-inflammatory drugs (NSAIDs) to promote the resolution of inflammation.

In addition to the lipoxins, a parallel role for n-3 very-long-chain fatty acid (VLCFA) mediators has been identified. These fall into two distinct classes; resolvins (resolution-phase interaction products) and protectins (stemming from initial protection of neural tissue also referred to as the neuroprotectins). Resolvins originating from EPA are referred to as the E-series, with resolvin E1 (RvE1) as the prototypical member, while resolvins originating from DHA represent the D series, typified by resolvin D1 (RvD1). However, DHA can also give rise to the protectins, such as neuroprotectin D1 (Serhan C N. Novel chemical mediators in the resolution of inflammation: resolvins and protectins. Anesthesiol. Clin. 2006, 24:341-64; Serhan C N. Novel eicosanoid and docosanoid mediators: resolvins, docosatrienes, and neuroprotectins. Curr. Opin. Clin. Nutr. Metab. Care 2005, 8:115-21; Serhan C N, Gotlinger K, Hong S, et al. Anti-inflammatory actions of neuroprotectin D1/protectin D1 and its natural stereoisomers: assignments of dihydroxy-containing docosatrienes. J. Immunol. 2006, 176:1848-59). Like lipoxins, both E and D series resolvins can also be triggered by aspirin (Serhan C N, Hong S, Gronert K, et al. Resolvins: a family of bioactive products of omega-3 fatty acid transformation circuits initiated by aspirin treatment that counter proinflammation signals. J. Exp. Med. 2002, 196:1025-37).

Given the central role of inflammation in many diseases, the identification of endogenous metabolic systems involved in inflammation control are of paramount interest. The inability to sufficiently “resolve” acute inflammation is the leading theory behind the establishment of chronic inflammatory states which underlie conditions such as cancer and Alzheimer's Disease. Of particular relevance is the effect of pro-resolution mediators on intestinal inflammatory conditions such as inflammatory bowel disease (IDB), Crohn's Disease, Colitis, and colon cancer.

Both RvE1 and LXA4 have been implicated with protective effects against colonic inflammation. RvE1 was shown to protect against the development of 2,4,6-trinitrobenzene sulfonic acid-induced colitis in mice, accompanied by a block in leukocyte infiltration, decreased proinflammatory gene expression, induced nitric oxide synthase, with improvements in survival rates and sustained body weight (Arita M, Yoshida M, Hong S, et al. Resolvin E1, an endogenous lipid mediator derived from omega-3 eicosapentaenoic acid, protects against 2,4,6-trinitrobenzene sulfonic acid-induced colitis. Proc. Natl. Acad. Sci. USA 2005, 102:7671-6). Similarly, LXA4 analogues have been shown to attenuate chemokine secretion in human colon ex vivo (Goh J, Baird A W, O'Keane C, et al. Lipoxin A(4) and aspirin-triggered 15-epi-lipoxin A(4) antagonize TNF-alpha-stimulated neutrophil-enterocyte interactions in vitro and attenuate TNF-alpha-induced chemokine release and colonocyte apoptosis in human intestinal mucosa ex vivo. J. Immunol. 2001, 167:2772-80), and attenuated 50% of genes, particularly those regulated by NFκB, induced in response to pathogenically induced gastroenteritis (Gewirtz A T, Collier-Hyams L S, Young A N, et al. Lipoxin a4 analogs attenuate induction of intestinal epithelial proinflammatory gene expression and reduce the severity of dextran sodium sulfate-induced colitis. J. Immunol. 2002, 168:5260-7). In vivo, LXA4 analogues reduced intestinal inflammation in DSS-induced inflammatory colitis, resulting in significantly reduced weight loss, hematochezia and mortality (Gewirtz A T, Collier-Hyams L S, Young A N, et al. Lipoxin a4 analogs attenuate induction of intestinal epithelial proinflammatory gene expression and reduce the severity of dextran sodium sulfate-induced colitis. J. Immunol. 2002, 168:5260-7).

Structurally, lipoxins, resolvins and protectins are mono-, di- and tri-hydroxylated products of the parent VLCFAs, catalyzed by various lipoxygenases, cyclooxygenases and p450 enzymes. While certain physiological effects of these molecules have been documented as discussed above, mechanisms describing how these resolution “stop signals” are exerted remain a mystery.

The inventors describe herein a novel class of hydroxylated fatty acids which play a role in reducing inflammation and promotion of pro-apoptotic, anti-cancer activity. These hydroxylated fatty acids are also useful biomarkers for diagnosing diseases and physiological conditions.

### SUMMARY OF THE INVENTION

It is an object of the invention to provide compounds for the treatment and mitigation of inflammation, inflammatory disorders and cancer, as well as related pharmaceutical compositions and methods of treatment.

It is also an object of the invention to provide compounds useful for detecting inflammation, inflammatory disorders and cancer in a subject, as well as related methods of detection or diagnosis.

According to an aspect of the present invention there is provided a compound of formula (I):

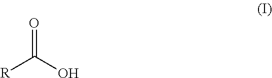

wherein R represents a hydroxy substituted C₂₄-C₄₀ straight chain aliphatic group containing at least one double bond in the carbon chain; and at least one carbon in the chain is substituted with a hydroxy group.

In the above formula (I), R may preferably be a C₂₈-C₃₆ aliphatic group, more preferably a C₂₈ aliphatic group. By straight chain aliphatic group, as used herein, it is meant an open chain saturated hydrocarbon, as, for example, an olefinic or alkenyl group. By hydroxy substituted, as used herein, it is meant that the compound may have one or more hydroxy substituents, in replacement of one or more hydrogen atoms in the hydrocarbon chain.

Without wishing to be limiting in any way, the above compound may in certain embodiments be one of the following compounds:

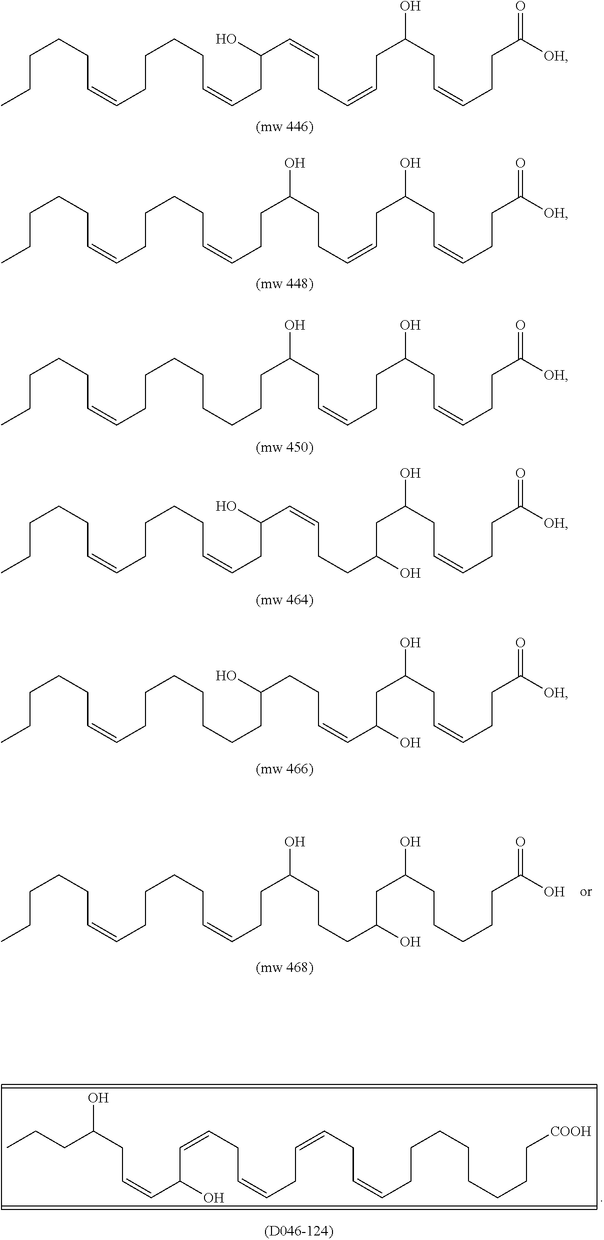

The above compound may be isolated from natural sources, or synthesized chemically. In addition, all compounds can be provided as a single stereoisomer or as a mixture thereof and/or as a pharmaceutically acceptable salt or ester thereof.

The above compound may also be labeled to facilitate use as a standard, for instance in diagnostic assays, in quantitation of analyte levels in vivo, and the like. In a non limiting embodiment, the compound is labeled with a stable isotope such as ¹³C, a radioisotope such as ³²P or ³⁵S, fluorescent tag such as fluorescein or equivalent. In an alternate non-limiting embodiment, the compound is labeled with or conjugated to an enzyme or protein, such as horse radish peroxidase (HRP), alkaline phosphatase, biotin, or the like, so as to facilitate detection in vitro or in vivo.

Thus, the invention further provides a standard comprising a compound of formula (I), labeled with a detection agent.

A kit comprising the above-described standard is also provided. Such a kit may comprise instructions and other materials useful for quantitating an analyte, or for performing a diagnostic assay as described herein.

As another aspect of the invention, there is provided a method of treating a subject diagnosed with CRC, or suspected of having CRC, comprising administering a compound of formula (I) in an amount sufficient to treat, prevent or mitigate the disease.

There is additionally provided a method of inhibiting tumor growth, comprising administering a compound of formula (I) in an amount sufficient to inhibit growth of the tumor. In certain embodiments, inhibition of tumor growth may include various degrees of tumor growth retardation including complete inhibition of growth. Such treatment may also involve a reduction in tumor size. Tumors may include, but are not limited to, cancers of the large intestine and rectum, such as adenocarcinomas, gastric and stomach cancers, pancreatic cancers, ovarian cancer, esophageal cancer, and other gastro-intestinal/abdominal cancers.

The invention further provides a method of treating or preventing a gastrointestinal (GI) disorder in a subject, comprising administering a compound of formula (I) to the subject in an amount sufficient to treat, prevent or mitigate the disease in the subject. The GI disorder may be a non-malignant disorder such as inflammatory bowel disease (IBD), Crohn's, and/or colitis, or the presence of polyps or various-grade dysplasias.

Also provided herein is a method of preventing inflammation and/or an inflammation-related disorder in a subject in need thereof, comprising administering a compound of formula (I) to the subject in an amount effective to prevent said inflammation and/or inflammation-related disorder.

As mentioned above, the invention also relates to methods of diagnosis and detecting disease, including early signs of disease. Accordingly, there is further provided herein a method for diagnosing a subject's CRC health state or change in health state, or for diagnosing CRC or the risk of CRC in a subject, comprising steps of:

a) analyzing a sample from the subject to quantify the amount of a compound of formula (I) in said sample;

b) comparing the quantified amount of the compound in the subject sample to a corresponding amount of the compound in one or more than one reference sample to determine the presence or absence of an increase or decrease in the amount of the compound in the subject sample; and

c) using said increase or decrease for diagnosing the subject's CRC health state or change in health state, or for diagnosing CRC or the risk of CRC in the subject.

The invention further relates to a method of identification and/or diagnosis of a subject having a hPULCFA deficiency disorder (hPDD), comprising measuring levels of a compound of formula (I) in the subject and comparing said levels to a corresponding standard level of hydroxylated polyunsaturated ultra long-chain fatty acids (hPULCFAs) in a normal state. Such a method may include steps of:

a) analyzing a sample from the subject to quantify the amount of a compound of formula (I) in the sample;

b) comparing the quantified amount of the compound in the subject sample to a corresponding amount of the compound in one or more than one reference sample to determine the presence or absence of an increase or decrease in the amount of the compound in the subject sample; and

c) using the increase or decrease for diagnosing hPDD in the subject.

The invention further relates to a method of treating hPDD in a subject by administering a compound of formula (I) in an amount sufficient to ameliorate the hPDD in the subject. Preferably the amount of compound administered is effective to elevate hPULCFA levels, and more preferably restore hPULCFA levels to a normal state.

By a ‘normal state’ is meant the level of hPULCFAs in subjects considered to be healthy or otherwise which do not have hPDD.

The present invention also relates to the use of one or more compounds of formula (I) as markers of inflammation, and for monitoring the effects of anti-inflammatory drugs. Thus, methods are provided which include steps of:

a) analyzing a sample from the subject to quantify the amount of a compound of formula (I) in the sample;

b) comparing the quantified amount of the compound in the subject sample to a corresponding amount of the compound in one or more than one reference sample to determine the presence or absence of an increase or decrease in the amount of the compound in the subject sample; and

c) using the increase or decrease for diagnosing inflammation or an inflammatory disease in the subject.

In the above method of diagnosing inflammation or an inflammatory disease, the inflammation may be caused by, or the inflammatory disease may include a GI disorder such as IBD, Crohn's, and/or colitis. Thus, such a method may encompass a method of diagnosing such GI disorders.

A method of monitoring the effect of an anti-inflammatory drug includes:

a) analyzing a sample from a subject treated with said anti-inflammatory drug to quantify the amount of a compound of formula (I) in the sample; and

b) comparing the quantified amount of the compound in the subject sample to a corresponding amount of the compound in one or more than one reference sample to determine the presence or absence of an increase or decrease in the amount of the compound in the subject sample;

wherein an increase or decrease in the amount of the compound in the subject sample indicates an effect caused by the anti-inflammatory drug in the subject.

In the above method, the subject treated with said anti-inflammatory drug will typically be diagnosed with or suspected to have an inflammation and/or an inflammatory condition or disease. Alternatively, the method may be applied in a comparative analysis including a group of subjects, including a first sub-group or population diagnosed with or suspected to have an inflammation and/or an inflammatory condition or disease, and a second sub-group or population diagnosed not to have or which do not exhibit physiological signs of an inflammation and/or an inflammatory condition or disease.

In certain embodiments of the above diagnostic methods, the sample from the subject is analyzed in step a) by mass spectrometry to obtain accurate mass intensity data for the compound, and the accurate mass intensity data is compared in step b) to corresponding accurate mass intensity data obtained from the one or more than one reference sample to identify an increase or decrease in accurate mass intensity. In addition, the sample from the subject may be further analyzed to quantify or obtain accurate mass intensity data for one or more than one internal control metabolite. In such embodiments a ratio can be determined between the quantified amount of the compound, or the accurate mass intensities obtained, to the quantified amount or accurate mass intensities obtained for the one or more than one internal control metabolite. The comparing step (b) then comprises comparing each ratio to one or more corresponding ratios obtained for the one or more than one reference sample.

In the above-described diagnostic methods, quantifying data may be obtained using a Fourier transform ion cyclotron resonance, time of flight, orbitrap, quadrupole or triple quadrupole mass spectrometer. Other methods of quantitating an analyte, including but not limited to tandem mass spectrometry, NMR or enzyme-linked immunosorbent assay (ELISA) methods may also be used. In addition, the sample can be any biological sample from the subject, preferably a blood sample, a blood serum sample, a cerebral spinal fluid sample or the like. Further, the accurate mass intensities represent ionized metabolites within a sample obtained by extraction methods as described herein, for example by performing a liquid/liquid extraction on the sample whereby non-polar metabolites are dissolved in an organic solvent and polar metabolites are dissolved in an aqueous solvent. In this way, the accurate mass intensities can be obtained from the ionization of the extracted samples using an ionization method such as positive electrospray ionization, negative electrospray ionization, positive atmospheric pressure chemical ionization, negative atmospheric pressure chemical ionization, or combinations of these methods.

A reference sample as referred to herein may include one or more than one reference sample, and will be selected based on the disease or condition being tested. For instance, when testing a subject's CRC health state or change in health state, or for diagnosing CRC or the risk of CRC in a patient, the one or more than one reference sample will be from one or more healthy individuals that have not been diagnosed with CRC and/or that do not exhibit physiological conditions associated with CRC. When testing a subject for hPDD, the one or more than one reference sample will be from one or more healthy individuals that have not been diagnosed with hPDD, that have hPULCFA levels consistent with the levels of the general population, and/or that do not exhibit physiological conditions associated with hPDD. For methods of diagnosing inflammation or an inflammatory disease, the one or more than one reference sample will be from one or more healthy individuals that have not been diagnosed with inflammation or an inflammatory disease and/or that do not exhibit physiological conditions associated with inflammation or an inflammatory disease. When monitoring the effect of an anti-inflammatory drug on the other hand, the one or more than one reference sample can be a sample from the same subject taken prior to administration of or treatment with the anti-inflammatory drug, or may alternatively be a sample from one or more healthy individuals diagnosed not to have or which do not exhibit physiological signs of an inflammation and/or an inflammatory condition or disease.

Also provided herein are the following compounds:

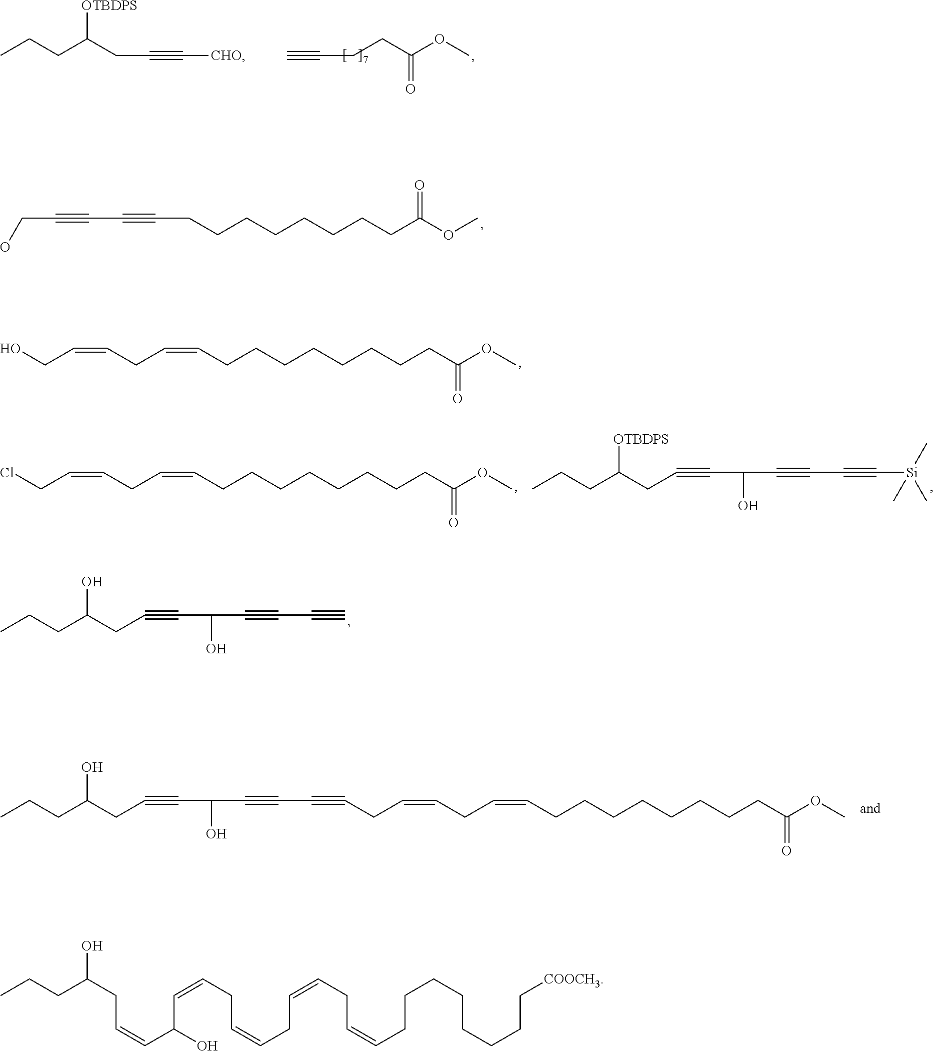

Such compounds are particularly useful as intermediates in the synthesis of compound D046-124:

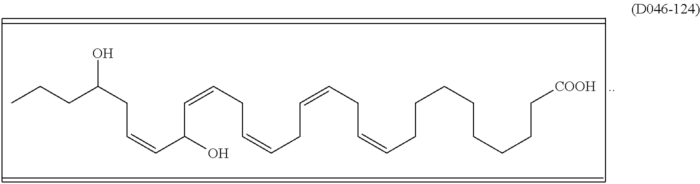

There is also provided herein a method of preparing the compound D046-124 comprising the following steps:

(i) reacting a compound of formula (II):

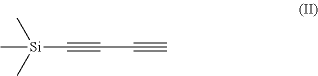

with a compound of formula (III):

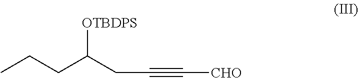

under conditions to produce a compound of formula (IV):

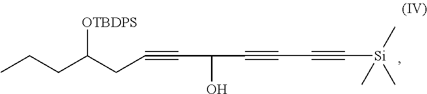

(ii) removing the TBDPS group to produce a compound of formula (V):

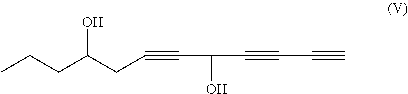

(iii) reacting the compound of formula (V) with a compound of formula (VI):

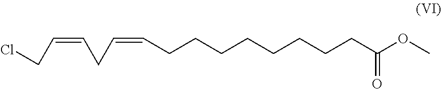

under conditions to produce a compound of formula (VII):

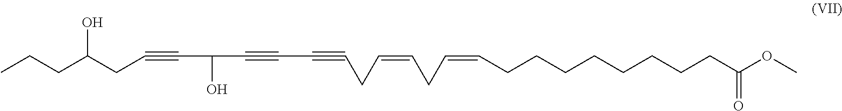

(iv) reacting the compound of formula (VII) with a catalyst under conditions to produce a compound of formula (VIII):

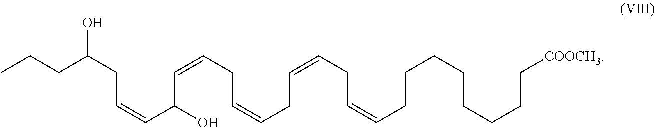

and
(v) hydrolyzing the terminal ester functional group of the compound of formula (VIII) to a carboxylic acid group thereby producing the title compound.

The above method may further comprise one or more purification steps to isolate compound D046-124. In addition, the compound of formula (VII) may in certain non-limiting embodiments be reacted in the above step (iv) in the presence of a Pd catalyst with calcium carbonate under hydrogen at 1 Atm pressure to selectively convert triple bonds to double bonds and thereby produce the compound of formula (VIII).

Those skilled in the art will recognize, or be able to ascertain using no more than routine experimentation, numerous equivalents to the specific procedures described herein. Such equivalents are considered to be within the scope of this invention and are covered by the following claims.

### BRIEF DESCRIPTION OF THE DRAWINGS

These and other features of the invention will become more apparent from the following description in which reference is made to the following figures.

FIG. 1. Study design. The study comprised three phases: FTICR-MS metabolomic discovery in three independent sample sets, structural investigation and determination of metabolic biomarkers as hPULCFAs, and validation using a triple-quadrupole MRM targeted assay.

FIG. 2. Scatter plots of average sample peak intensity fold change between CRC and normal patient sera in three independent studies. Sample-specific peaks for all subjects were log 2 normalized to the mean of the control population, and plotted according to mass (Da). Points are colored according to significance based on an unpaired student's t-test (see legend). (A) GCI discovery population, (B) Seracare 1 discovery population, (C) Osaka discovery population. The region boxed in grey represents the cluster of masses between 440 and 600 Da consistently reduced in CRC patients compared to controls in all three cohorts.

FIG. 3. Relative intensities of metabolites 446 and 448 by disease stage and AUCs for each discovery dataset. (A) Bar charts of relative intensity versus disease stage in each sample set; (B) summary of P-value comparisons between disease stages and controls for metabolites 446 and 448; (C) ROC analysis based on markers 446 and 448 and all CRCs versus all controls in each discovery set.

FIG. 4. Extracted mass spectrum of serum from normal subjects and CRC patients. Extracts from five representative CRC and five control samples from the GCI discovery set were subjected to high performance liquid chromatography (HPLC) followed by full-scan detection on an Applied Biosystems QSTAR XL™ mass spectrometer in APCI negative mode. The average intensities of all ions within the mass range 100 to 700 Da eluting between 16 and 18 minutes are shown for each cohort. The boxed region indicates spectral features present in normal patients but absent from CRC-positive serum.

FIG. 5. Results of triple-quadrupole MRM analysis of Seracare 2 validation sample set. (A) Scatter plots of the concentrations of hPULCFAs 446, 448 and 450 expressed as ¹³C-cholic acid equivalents in asymptomatic controls, and pre-treatment CRC patients, (B) ROC analysis based upon the corresponding scatter plots in (A). Grey dotted lines indicate the 95% confidence interval. (C) bar charts of the average concentration equivalents of hPULCFAs by disease stage. Error bars represent standard errors of the mean. (D) ROC analysis by disease stage.

FIG. 6. Results of triple-quadrupole MRM analysis of the Chiba validation sample set. (A) Scatter plots of the concentrations of hPULCFAs 446, 448 and 450 expressed as ¹³C-cholic acid equivalents in asymptomatic controls and pre-treatment CRC patients (B) ROC analysis based upon the corresponding scatter plots in (A). Grey dotted lines indicate the 95% confidence interval. (C) bar charts of the average concentration equivalents of hPULCFAs by disease stage. Error bars represent standard errors of the mean. (D) ROC analysis by disease stage.

FIG. 7. MS/MS spectra for biomarker m/z 446.

FIG. 8. MS/MS spectra for biomarker m/z 448.

FIG. 9. MS/MS spectra for biomarker m/z 450.

FIG. 10. MS/MS spectra for biomarker m/z 464.

FIG. 11. MS/MS spectra for biomarker m/z 466.

FIG. 12. MS/MS spectra for biomarker m/z 468.

FIG. 13. Purification process to obtain hPULCFA enriched fractions from human serum. Dried organic extracts of serum were initially purified by reversed phase flash column chromatography using water/acetonitrile step solvent gradient to obtain semi purified hPULCFA enriched fraction (F9). Several of F9s were combined for a secondary purification step by normal phase flash column chromatography using hexane/chloroform/methanol step solvent gradient to obtain highly hPULCFA enriched fraction 7 (F7_{—}2).

FIG. 14. LC/MS spectra of Stage I fraction 9 (F9) containing a mixture of fatty acids and colorectal cancer biomarkers obtained after fractionating serum extract on reverse phase column.

FIG. 15. LC/MS spectra of Stage II fraction 7 (F7) containing approximately 65% enrichment in CRC biomarkers.

FIG. 16. Total ion chromatogram of unpurified human serum extract (A); extracted mass spectra of all ions (B); and extracted mass spectra of ions between 440 and 520 Da (C).

FIG. 17. Total ion chromatogram of human hPULCFA-negative serum extract following the enrichment procedure described herein (A); extracted mass spectra (B). No hPULCFAs are present.

FIG. 18. Total ion chromatogram of human hPULCFA-positive serum extract following the enrichment procedure described herein (A); extracted mass spectra (B). hPULCFAs are present between 440 and 600 Da.

FIG. 19. Cell proliferation assay of SW620 colon cancer cells treated with varying doses of total serum extract (as shown in FIG. 16) for 48 hours.

FIG. 20. Bright field examination of cells treated with hPUCLFA-enriched extracts. MCF-7 cells were treated for 24 hours with 80 ug/ml of semi-purified extracts enriched for (hPULCFA+ve) or depleted of (hPULCFA−ve) hPULCFAs, vehicle or 1 uM doxorubicin and imaged with inverted light microscopy. An enlargement of the cells are shown in the top left of each panel. A significant effect on cellular viability and morphology is evident with the hPULCFA+ve treatment (bottom left) compared to the other treatments.

FIG. 21. Western (immunoblot) of MCF7 cell lysates for the caspase-mediated pro-apoptotic Poly-ADP-Ribose Polymerase (PARP) cleavage fragment following treatment with hPULCFA+ve and −ve extracts (80 ug/ml).

FIG. 22. Cellular proliferation rates of SW620 colon cancer cells following treatment with 80 ug/ml hPUCLFA-positive, hPULCFA-negative, and vehicle for 12, 24 and 48 hours.

FIG. 23. Western (immunoblot) of SW620 cell lysates for the caspase-mediated pro-apoptotic Poly-ADP-Ribose Polymerase (PARP) cleavage fragment following treatment with hPULCFA+ve and −ve extracts (80 ug/ml).

FIG. 24. Western (immunoblot) of SW620 cell lysates for the pro-inflammatory transcription factor NFκB following treatment with hPULCFA+ve and −ve extracts (80 ug/ml).

FIG. 25. Western (immunoblot) of SW620 cell lysates for the NFκB negative regulatory protein IκBα following treatment with hPULCFA+ve and −ve extracts (80 ug/ml).

FIG. 26. Western (immunoblot) of SW620 cell lysates for inducible nitric oxide synthase (iNOS or NOS2) following treatment with hPULCFA+ve and −ve extracts (80 ug/ml).

FIG. 27. Levels of nitrite as an indicator of nitric oxide production in conditioned media following treatment of SW620 cells with hPULCFA+ve and −ve extracts (80 ug/ml) using the Griess reagent system.

FIG. 28. Relative TNFα mRNA transcript levels, based on quantitative real-time rtPCR, following pre-treatment of 1 ug/ml LPS-stimulated RAW293 macrophage cells with hPULCFA+ve and −ve extracts. Triangles represent increasing doses of 20, 40 and 80 ug/ml. *p<0.05 versus +LPS treatment alone.

FIG. 29. Relative TNFα cell lysate protein levels, as determined by ELISA, following pre-treatment of 1 ug/ml LPS-stimulated RAW293 macrophage cells with hPULCFA+ve and −ve extracts (80 ug/ml). *p<0.05 versus +LPS treatment alone.

FIG. 30. Relative TNFα protein levels in conditioned media, as determined by ELISA, following pre-treatment of 1 ug/ml LPS-stimulated RAW293 macrophage cells with hPULCFA+ve and −ve extracts (80 ug/ml). *p<0.05 versus +LPS treatment alone.

FIG. 31. Relative iNOS mRNA transcript levels, based on quantitative real-time rtPCR, following pre-treatment of 1 ug/ml LPS-stimulated RAW293 macrophage cells with hPULCFA+ve and −ve extracts. Triangles represent increasing doses of 20, 40 and 80 ug/ml. *p<0.05 versus +LPS treatment alone.

FIG. 32. Relative iNOS protein levels in cell lysates, as determined by Western Blot, following pre-treatment of 1 ug/ml LPS-stimulated RAW293 macrophage cells with hPULCFA+ve and −ve extracts (80 ug/ml). ns, non-specific.

FIG. 33. Relative levels of nitrite as an indicator of nitric oxide production in conditioned media following treatment of 1 ug/ml LPS-stimulated RAW293 macrophage cells with hPULCFA+ve and −ve extracts (80 ug/ml) using the Griess reagent system. *p<0.05 versus +LPS treatment alone.

FIG. 34. Relative COX2 mRNA transcript levels, based on quantitative real-time rtPCR, following pre-treatment of 1 ug/ml LPS-stimulated RAW293 macrophage cells with hPULCFA+ve and −ve extracts. Triangles represent increasing doses of 20, 40 and 80 ug/ml. *p<0.05 versus +LPS treatment alone.

FIG. 35. Relative IL-1β mRNA transcript levels, based on quantitative real-time rtPCR, following pre-treatment of 1 ug/ml LPS-stimulated RAW293 macrophage cells with hPULCFA+ve and −ve extracts. Triangles represent increasing doses of 20, 40 and 80 ug/ml. *p<0.05 versus +LPS treatment alone.

FIG. 36. Relative IL-1β protein levels in cell lysates, as determined by ELISA, following pre-treatment of 1 ug/ml LPS-stimulated RAW293 macrophage cells with hPULCFA+ve and −ve extracts (80 ug/ml). *p<0.05 versus +LPS treatment alone.

FIG. 37. Relative TNFα transcript levels, as determined by quantitative real-time rtPCR, following pre-treatment of 1 ug/ml LPS-stimulated RAW293 macrophage cells with various concentrations of pure synthetic hPULCFA D046-124. *p<0.05 versus +LPS treatment alone.

FIG. 38. Relative TNFα protein levels in conditioned media, as determined by ELISA, following pre-treatment of 1 ug/ml LPS-stimulated RAW293 macrophage cells with 0.5 and 1 mM pure synthetic hPULCFA D046-124.

FIG. 39. Relative iNOS transcript levels, as determined by quantitative real-time rtPCR, following pre-treatment of 1 ug/ml LPS-stimulated RAW293 macrophage cells with various concentrations of pure synthetic hPULCFA D046-124. *p<0.05 versus +LPS treatment alone.

FIG. 40. Relative levels of nitrite as an indicator of nitric oxide production in conditioned media following treatment of 1 ug/ml LPS-stimulated RAW293 macrophage cells with various concentrations of pure synthetic hPULCFA D046-124. *p<0.05 versus +LPS treatment alone.

FIG. 41. Relative IL-10 protein levels in conditioned media, as determined by ELISA, following pre-treatment of 1 ug/ml LPS-stimulated RAW293 macrophage cells with various concentrations of pure synthetic hPULCFA D046-124. *p<0.05 versus +LPS treatment alone.

FIG. 42. Seracare Pre-Treatment NSAID Effects on six hPULCFAs.

FIG. 43. Bioserve Post-Treatment NSAID Effects on six hPULCFAs.

FIG. 44. Reduction of TNF-alpha levels in hPULCFA-positive extract following LPS induction.

FIG. 45. Reduction of LPS-induced nitric oxide synthase (NOS2) in hPULCFA-positive extract. Top pane: Western blotting analysis; bottom pane: Ponceau S stained gel.

FIG. 46. Dose-dependent reduction of nitrite levels in conditioned media of cells treated with hPULCFA positive extract.

FIG. 47. NMR spectra for Compound 2.

FIG. 48. NMR spectra for Compound 2.

FIG. 49. NMR spectra for Compound 3.

FIG. 50. NMR spectra for Compound 4.

FIG. 51. NMR spectra for Compound 5.

FIG. 52. NMR spectra for Compound 6.

FIG. 53. NMR spectra for Compound 7.

FIG. 54. LC chromatograph for Compound 7.

FIG. 55. MS spectra for Compound 7.

FIG. 56. NMR spectra for Fragment A.

FIG. 57. LC chromatograph for Fragment A.

FIG. 58. MS spectra for Fragment A.

FIG. 59. NMR spectra for Compound 9.

FIG. 60. NMR spectra for Compound 9.

FIG. 61. MS spectra for Compound 9.

FIG. 62. IR absorption spectra for Fragment B.

FIG. 63. NMR spectra for Fragment B.

FIG. 64. MS spectra for Fragment B.

FIG. 65. NMR spectra for Compound 11.

FIG. 66. NMR spectra for Compound 11.

FIG. 67. NMR spectra for Compound 12.

FIG. 68. LC chromatograph for Compound 12.

FIG. 69. MS spectra for Compound 12.

FIG. 70. NMR spectra for Compound 13.

FIG. 71. LC chromatograph for Compound 13.

FIG. 72. MS spectra for Compound 13.

FIG. 73. NMR spectra for Fragment C.

FIG. 74. NMR spectra for Fragment C.

FIG. 75. LC chromatograph for Fragment C.

FIG. 76. MS spectra for Fragment C.

FIG. 77. NMR spectra for Compound 15.

FIG. 78. LC chromatograph for Compound 15.

FIG. 79. MS spectra for Compound 15.

FIG. 80. NMR spectra for Compound 16.

FIG. 81. NMR spectra for Compound 16.

FIG. 82. MS spectra for Compound 16.

FIG. 83. NMR spectra for Compound 17.

FIG. 84. LC chromatograph for Compound 17.

FIG. 85. MS spectra for Compound 17.

FIG. 86. NMR spectra for Compound 18.

FIG. 87. LC chromatograph for Compound 18.

FIG. 88. MS spectra for Compound 18.

FIG. 89. LC chromatograph for Compound D046-124 (Also referred to herein as GVK-FFS-09-06-PHM).

FIG. 90. MS spectra for Compound D046-124.

### DETAILED DESCRIPTION

Until now there have been no accurate serum markers for detecting early risk of colorectal cancer (CRC). To address this need, a mass spectrometry-based discovery platform was used to identify metabolic biomarkers within the serum metabolomes of treatment-naive CRC patients. The “non-targeted” approach has the advantage of detecting novel compounds, and was therefore ideally suited for a biomarker-driven approach. The use of a mass spectrometry-based discovery platform also has the added advantage of being readily translated into a quantitative diagnostic method based upon triple-quadruple multiple-reaction-monitoring (TQ-MRM).

Biomarker discovery was performed using Fourier transform ion cyclotron resonance mass spectrometry (FTICR-MS). Comprehensive metabolic profiles of CRC patients and controls from three independent populations from different continents (USA and Japan; total n=222) were obtained and the best inter-study biomarkers determined. Structural characterization of these and related markers was performed using MS/MS and NMR technologies. Commercial clinical utility evaluations were performed using a targeted high-throughput triple-quadrupole MRM (TQ-MRM) method for three biomarkers in two further independent populations from the USA and Japan (total n=220).

These comprehensive metabolomic analyses revealed significantly reduced levels of C28-C36 hydroxylated polyunsaturated ultra long-chain fatty-acids (hPULCFAs) in all three independent cohorts of CRC patient samples relative to controls. Structure elucidation studies on the C28 molecules revealed two families harboring either two hydroxyl substitutions (446, 448 and 450) or three hydroxyl substitutions (464, 466 and 468) and varying degrees of unsaturation. The TQ-MRM method successfully reproduced the FTMS results in two further independent studies. In total, two biomarkers in five independent populations across two continental regions were evaluated (three by FTICR-MS and two by TQ-MRM). The ten resultant receiver-operator characteristic curve AUCs ranged from 0.85 to 0.98 (average=0.91±0.04).

Systemic metabolic dysregulation of these previously unknown metabolites was found to be highly associated with the presence of CRC. The metabolites are measurable in biological samples such as serum, and a decrease in their concentration is highly sensitive and specific for the presence of CRC regardless of ethnic or geographic background. The measurement of these metabolites therefore provides a useful tool for the early detection and screening of CRC.

In addition to being useful CRC biomarkers, the hPULCFAs described herein reduce cell proliferation and have been shown to play a role in promoting apoptosis. The hPULCFAs also have anti-inflammatory activity, as demonstrated through investigations using a series of inflammatory proteins including NFκB, IκBα, NOS2, COX2, TNF-alpha and SOD, as well as by measuring nitrite levels in media of conditioned cells. Anti-inflammatory activity was also demonstrated through clinical testing of CRC and healthy subjects taking NSAIDs, whereby the use of NSAIDs resulted in the increase of hPULCFA levels in deficient subjects.

Taken all together, the inventors have provided a broad range of support for the use of the described hPULCFAs in therapeutic and diagnostic applications which will be discussed in further detail below.

Accordingly, there is herein provided a compound according to formula (I):

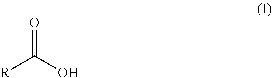

wherein R represents a hydroxy substituted C₂₄-C₄₀ straight chain aliphatic group containing at least one double bond in the carbon chain; and at least one carbon in the chain is substituted with a hydroxy group.

In the above formula (I), R may include a C₂₄-C₄₀ straight chain aliphatic group containing any number of C atoms from 24 to 40, including 24, 25, 26, 27, 28, 29, 30, 31, 32, 33, 34, 35, 36, 37, 38, 39 and 40. Preferably, the hydrocarbon chain is a C₂₈-C₃₆ aliphatic group, especially a C₂₈ aliphatic group. By straight chain aliphatic group, as used herein, it is meant an open chain saturated hydrocarbon, as, for example, an olefinic or alkenyl group. By hydroxy substituted, as used herein, it is meant that the compound may have one or more hydroxy substituents, in replacement of one or more hydrogen atoms in the hydrocarbon chain.

As noted above, at least one carbon in the hydrocarbon chain of R is substituted with a hydroxy (OH) group. The number of OH substitutions in the chain may be any number from 1 to 10, including 1, 2, 3, 4, 5, 6, 7, 8, 9 or 100H substitutions along the length of the fatty acyl chain. However, it may be preferred in some embodiments for there to be fewer OH substitutuents, for instance from 1 to 4, and especially 2 or 30H substitutuents. The positioning of these OH substituents along the length of the acyl chain may be varied, such as at carbon C1, C2, C3, C4, C5, C6, C7, C8, C9, C10, C11, C12, C13, C14, C15, C16, C17, C18, C19, C20, C21, C22, C23, C24, or for longer chains at C25, C26, C27, C28, C29, C30, C31, C32, C33, C34, C35, C36, C37, C38, C39, C30, and including combinations thereof.

The number of double bonds in the above compound will generally depend upon the length of the fatty acyl chain and is therefore limited by the number of C atoms. Thus, the number of double bonds could be 1, 2, 3, 4, 5, 6, 7, 8, 9, 10, 11, 12, 13, 14, 15, 16, 17, 18, 19 or 20, and more preferably from 3 to 6 double bonds positioned variably along the length of the acyl chain.

The above compound may be isolated from natural sources, or synthesized chemically. The compounds may also be produced through a bio-engineered approach, for example, by the use of genetically engineered bacterial or mammalian cell cultures (or bioreactors) containing the metabolic enzymes required synthesize the compounds.

The described compounds can also be provided in pharmaceutical compositions together with an acceptable carrier or excipient, or together with one or more separate active agents or drugs as part of a pharmaceutical combination. In addition, the pharmaceutical compositions may be administered in a treatment regime with other drugs or pharmaceutical compositions, either separately or in a combined formulation or combination.

Combinations of compounds of formula (I) are also provided herein. Such combinations may be especially useful due to synergies or additive effects of the various compounds in the combination or mixture.

In addition, compounds of formula (I) or combinations comprising them may be prepared as supplements, nutraceuticals or prepared into functional foods with health benefits.

A composition of the present invention is preferably formulated with a vehicle pharmaceutically acceptable for administration to a subject, preferably a human, in need thereof. Methods of formulation for such compositions are well known in the art and taught in standard reference texts such as Remington's Pharmaceutical Sciences, Mack Publishing Co., Easton, Pa., 1985. A composition of the present invention may comprise a single compound, or a combination thereof.

Compositions of the present invention may be administered alone or in combination with a second drug or agent.

Formulations expected to be useful in the present invention, e.g., injectable formulations including intravenous formulations, may include, but are not limited to, sterile aqueous solutions (where water soluble) or dispersions and sterile powders for the extemporaneous preparation of sterile injectable solutions or dispersions. In all cases, the composition must be sterile and must be fluid to the extent that easy syringability exists. It must be stable under the conditions of manufacture and storage and must be preserved against the contaminating action of microorganisms such as bacteria and fungi. The vehicle can be a solvent or dispersion medium containing, for example, water, ethanol, polyol (for example, glycerol, propylene glycol, liquid polyethylene glycol, and the like), suitable mixtures thereof, and oils (e.g. vegetable oil). The proper fluidity can be maintained, for example, by the use of a coating such as lecithin, by the maintenance of the required particle size in the case of dispersion, and by the use of surfactants.

Prevention of the action of microorganisms can be achieved by various antibacterial and antifungal agents, for example, parabens, chlorobutanol, phenol, ascorbic acid, thimerosal, and the like. In some cases, it will be preferable to include isotonic agents, for example, sugars, sodium chloride, or polyalcohols such as mannitol and sorbitol, in the composition. Prolonged absorption of the injectable compositions can be brought about by including an agent in the composition that delays absorption, for example, aluminum monostearate or gelatin.

Sterile injectable solutions can be prepared by incorporating the composition of the present invention in the required amount in an appropriate solvent with one or a combination of ingredients enumerated above, as required, followed by filter sterilization. Generally, dispersions are prepared by incorporating the composition of the present invention into a sterile vehicle which contains a basic dispersion medium and the required other ingredients from those enumerated above. In the case of sterile powders for the preparation of sterile injectable solutions, the preferred methods of preparation are vacuum drying and freeze-drying which yield a powder of the compound of the invention, optionally plus any additional desired ingredient from a previously sterile-filtered solution thereof.

Solid dosage forms for oral administration of a compound of the present invention include, but are not limited to, ingestible capsules, tablets, pills, lollipops, powders, granules, elixirs, suspensions, syrups, wafers, sublingual or buccal tablets, troches, and the like. In such solid dosage forms the compound is mixed with at least one inert, pharmaceutically acceptable excipient or diluent or assimilable edible carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof, or incorporated directly into the subject's diet. In the case of capsules, tablets and pills, the dosage form may also comprise buffering agents. Solid compositions of a similar type may also be employed as fillers in soft and hard-filled gelatin capsules using such excipients as lactose or milk sugar as well as high molecular weight polyethylene glycols and the like. The percentage of the compound of the invention in the compositions and preparations may, of course, be varied. The amount of compound in such therapeutically useful compositions is such that a suitable dosage will be obtained.

The solid dosage forms of tablets, dragees, capsules, pills, and granules can be prepared with coatings and shells such as enteric coatings and other coatings well-known in the pharmaceutical formulating art. They may optionally contain opacifying agents and can also be of a composition that they release the compound(s) of the invention only, or preferentially, in a certain part of the intestinal tract, optionally, in a delayed manner. Examples of embedding compositions which can be used include polymeric substances and waxes. The compositions can also be in micro-encapsulated form, if appropriate, with one or more of the above-mentioned excipients.

Liquid dosage forms for oral administration include pharmaceutically acceptable emulsions, solutions, suspensions, syrups and elixirs. In addition to the compound of the invention, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethyl formamide, oils (in particular, cottonseed, ground nut corn, germ olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Suspensions, in addition to the compound of the invention, may contain suspending agents as, for example, ethoxylated isostearyl alcohols, polyoxyethylene sorbitol and sorbitan esters, microcrystalline cellulose, aluminum metahydroxide, bentonite, agar-agar, and tragacanth, and mixtures thereof.

Accordingly, the compositions of the present invention can be administered to a subject, preferably a mammal, more preferably a human, to treat and/or prevent disease. The compositions may be administered by various routes including, but not limited to, orally, intravenously, intramuscularly, intraperitoneally, topically, subcutaneously, rectally, dermally, sublingually, buccally, intranasally or via inhalation. The formulation and route of administration as well as the dose and frequency of administration can be selected routinely by those skilled in the art based upon the severity of the condition being treated, as well as patient-specific factors such as age, weight and the like.

One skilled in the art recognizes that interspecies pharmacokinetic scaling can be used to study the underlining similarities (and differences) in drug disposition among species, to predict drug disposition in an untested species, to define pharmacokinetic equivalence in various species, and to design dosage regimens for experimental animal models, as discussed in Mordenti, Man versus Beast: Pharmacokinetic Scaling in Mammals, 1028, Journal of Pharmaceutical Sciences, Vol. 75, No. 11, November 1986.

The above compounds and compositions can be used for the treatment of inflammation and inflammation-related disorders such as cancer. For instance, a compound of formula (I) or composition comprising such a compound may be administered to a subject diagnosed with CRC, or suspected of having CRC, in an amount sufficient to treat, prevent or mitigate the disease. Compounds of formula (I) may also be used to treat or prevent other non-malignant GI disorders such as IBD, Crohn's, and colitis.

The compounds and compositions may also be useful in preventative methods, for instance by administering a compound of formula (I) to a subject in a regimen to prevent inflammation and inflammation-related disorders such as cancer.

The compounds and compositions may also be used in a method of identification and diagnosis of subjects lacking hPULCFAs, referred to as hPULCFA Deficiency Disorder (hPDD). Such subjects may have elevated inflammatory risk, risk of the inability to sufficiently resolve acute inflammation, and/or disease states associated with inflammation. Similarly, the compounds and compositions can also be used to treat hPDD in a subject, whereby a compound of formula (I) or composition comprising the compound is administered in an amount sufficient to ameliorate the hPDD in the subject.

One or more compounds of formula (I) may also be used as markers of inflammation, and for monitoring the effects of anti-inflammatory drugs.

Biological samples used in the above methods can originate from anywhere within the body, for example but not limited to, blood (serum/plasma), cerebral spinal fluid (CSF), urine, stool, breath, saliva, or biopsy of any solid tissue including tumor, adjacent normal, smooth and skeletal muscle, adipose tissue, liver, skin, hair, brain, kidney, pancreas, lung, colon, stomach, or other. Of particular interest are samples that are serum or CSF. While the term “serum” is used herein, those skilled in the art will recognize that plasma or whole blood or a sub-fraction of whole blood may be used.

When a blood sample is drawn from a patient there are several ways in which the sample can be processed. The range of processing can be as little as none (i.e. frozen whole blood) or as complex as the isolation of a particular cell type. The most common and routine procedures involve the preparation of either serum or plasma from whole blood. All blood sample processing methods, including spotting of blood samples onto solid-phase supports, such as filter paper or other immobile materials, are also contemplated by the invention.

The processed blood sample described above is then further processed to make it compatible with the methodical analysis technique to be employed in the detection and measurement of the biochemicals contained within the processed serum sample. The types of processing can range from as little as no further processing to as complex as differential extraction and chemical derivatization. Extraction methods could include sonication, soxhlet extraction, microwave assisted extraction (MAE), supercritical fluid extraction (SFE), accelerated solvent extraction (ASE), pressurized liquid extraction (PLE), pressurized hot water extraction (PHWE) and/or surfactant assisted extraction (PHWE) in common solvents such as methanol, ethanol, mixtures of alcohols and water, or organic solvents such as ethyl acetate or hexane. The preferred method of extracting metabolites for HTS analysis is to perform a liquid/liquid extraction whereby non-polar metabolites dissolve in an organic solvent and polar metabolites dissolve in an aqueous solvent.

A step of analyzing the sample may comprise analyzing the sample using a mass spectrometer (MS). For example, and without wishing to be limiting, such mass spectrometer could be of the FTMS, orbitrap, time of flight (TOF) or quadrupole types. Alternatively, the mass spectrometer could be equipped with an additional pre-detector mass filter. For example, and without wishing to be limiting such instruments are commonly referred to as quadrupole-FTMS (Q-FTMS), quadrupole-TOF (Q-TOF) or triple quadrupole (TQ or QQQ). In addition, the mass spectrometer could be operated in either the parent ion detection mode (MS) or in MSn mode, where n>=2. MSn refers to the situation where the parent ion is fragmented by collision induced dissociation (CID) or other fragmentation procedures to create fragment ions, and then one or more than one of said fragments are detected by the mass spectrometer. Such fragments can then be further fragmented to create further fragments. Alternatively, the sample could be introduced into the mass spectrometer using a liquid or gas chromatographic system or by direct injection.

The extracted samples may be analyzed using any suitable method known in the art. For example, and without wishing to be limiting in any manner, extracts of biological samples are amenable to analysis on essentially any mass spectrometry platform, either by direct injection or following chromatographic separation. Typical mass spectrometers are comprised of a source which ionizes molecules within the sample, and a detector for detecting the ionized molecules or fragments of molecules. Non-limiting examples of common sources include electron impact, electrospray ionization (ESI), atmospheric pressure chemical ionization (APCI), atmospheric pressure photo ionization (APPI), matrix assisted laser desorption ionization (MALDI), surface enhanced laser desorption ionization (SELDI), and derivations thereof. Common mass separation and detection systems can include quadrupole, quadrupole ion trap, linear ion trap, time-of-flight (TOF), magnetic sector, ion cyclotron (FTMS), Orbitrap, and derivations and combinations thereof. The advantage of FTMS over other MS-based platforms is its high resolving capability that allows for the separation of metabolites differing by only hundredths of a Dalton, many which would be missed by lower resolution instruments.

By the term “metabolite”, it is meant specific small molecules, the levels or intensities of which are measured in a sample, and that may be used as markers to diagnose a disease state. These small molecules may also be referred to herein as “metabolite marker”, “metabolite component”, “biomarker”, or “biochemical marker”.

The metabolites are generally characterized by their accurate mass, as measured by mass spectrometry techniques used in the above methods. The accurate mass may also be referred to as “accurate neutral mass” or “neutral mass”. The accurate mass of a metabolite is given herein in Daltons (Da), or a mass substantially equivalent thereto. By “substantially equivalent thereto”, it is meant that a +/−5 ppm difference in the accurate mass would indicate the same metabolite, as would be recognized by a person of skill in the art. The accurate mass is given as the mass of the neutral metabolite. As would be recognized by a person of skill in the art, the ionization of the metabolites, which occurs during analysis of the sample, the metabolite will cause either a loss or gain of one or more hydrogen atoms and a loss or gain of an electron. This changes the accurate mass to the “ionized mass”, which differs from the accurate mass by the mass of hydrogens (or other adducts such as sodium, potassium, ammonia, and others known in the art) and electrons lost or gained during ionization. Unless otherwise specified, the accurate neutral mass will be referred to herein.

Similarly, when a metabolite is described by its molecular formula the molecular formula of the neutral metabolite will be given. Naturally, the molecular formula of the ionized metabolite will differ from the neutral molecular formula by the number of hydrogens (or other adducts such as sodium, potassium, ammonia, and others known in the art) lost or gained during ionization.

Data is collected during analysis and quantifying data for one or more than one metabolite is obtained. “Quantifying data” is obtained by measuring the levels or intensities of specific metabolites present in a sample.

The quantifying data is compared to corresponding data from one or more than one reference sample. The “reference sample” is any suitable reference sample for the particular disease state or condition. As would be understood by a person of skill in the art, more than one reference sample may be used for comparison to the quantifying data.

The step of analyzing the sample can be as described above. The one or more than one reference sample may be a first reference sample obtained from a control individual. The “internal control metabolite” refers to an endogenous metabolite naturally present in the subject or patient. Any suitable endogenous metabolite that does not vary over the disease state or condition can be used as the internal control metabolite.

Use of the ratio of the metabolite marker to the internal control metabolite can, in certain embodiments of the methods described herein, offer measurements that are more stable and reproducible than measurement of absolute levels of the metabolite marker. As the internal control metabolite is naturally present in all samples and does not appear to vary significantly over disease states, the sample-to-sample variability (due to handling, extraction, etc) is minimized.

### DEFINITIONS

The term “effective amount” means that amount of a compound, drug or pharmaceutical agent that will elicit the biological or medical response of a tissue, system, animal, or human that is being sought, for instance, by a researcher or clinician. Furthermore, the term “therapeutically effective amount” means any amount which, as compared to a corresponding subject who has not received such amount, results in improved treatment, healing, prevention, or amelioration of a disease, disorder, or side effect, or a decrease in the rate of advancement of a disease or disorder. The term also includes within its scope amounts effective to enhance normal physiological function.

“Hydroxyl” and “hydroxy” refers to —OH.

A “pharmaceutical agent” or “drug” refers to a chemical compound or composition capable of inducing a desired therapeutic or prophylactic effect when properly administered to a subject.

All chemical compounds include both the (+) and (−) stereoisomers, as well as either the (+) or (−) stereoisomer.

Other chemistry terms herein are used according to conventional usage in the art, as exemplified by The McGraw-Hill Dictionary of Chemical Terms (1985) and The Condensed Chemical Dictionary (1981).

The present invention will be further illustrated in the following examples.

### EXAMPLES

### 1. Reduced Serum Levels of hPULCFAs in CRC Patients

### Materials and Methods

### Patient Sample Selection

Clinical samples used for the first discovery project were obtained from Genomics Collaborative, Inc. (GCI), while samples for the second discovery project and one validation project were obtained from Seracare Lifesciences. These companies specialize in the collection and storage of serum and tissue samples specifically for research purposes. Samples were collected, processed and stored in a consistent manner by teams of physicians as part of a global initiative using standardized protocols and operating procedures. All samples were properly consented, and clinical protocols were approved by ethics review boards. The inclusion criterion for patient sample selection from the GCI and Seracare biobanks for both the discovery and validation cohorts was that the serum be taken prior to any form of treatment, including surgery, chemo, or radiation therapies. All samples were accompanied by detailed pathology reports which were independently verified by certified pathologists at GCI and Seracare. The GCI discovery sample set included serum samples from 40 pre-treatment CRC patients and 50 controls, the Seracare discovery set included samples from 26 pre-treatment CRC and 25 controls, and the validation Seracare set included 70 pretreatment CRC and 70 controls. The discovery samples provided by Osaka Medical University included 46 pre-surgery CRC patients 35 controls which were prospectively collected according to the standard collection protocol of the institution, and were properly consented. The samples for the Chiba Japan validation population, which included 40 pre-surgery CRC patients and 40 controls, were also prospectively collected under an ethics reviewed protocol and proper consent. A summary of the populations including disease staging is shown in Table 1. All samples were processed and analyzed in a randomized manner, and the results unblinded following analysis.

TABLE 1
Summary of case-control populations used in this study.

| | FTICR-MS discovery | | | | | | MRM validation | | | |
|---|---|---|---|---|---|---|---|---|---|---|
| | SCI | | SERACARE 1 | | OSAKA | | SERACARE 2 | | CHIBA | |
| | CRC | Control | CRC | Control | CRC | Control | CRC | Control | CRC | Control |
| Total | 40 | 50 | 26 | 25 | 46 | 35 | 70 | 70 | 40 | 40 |
| Male N | 19 | 24 | 17 | 16 | 27 | NA | 44 | 41 | 19 | 24 |
| Male age | 56 ± 13 | 56 ± 11 | 61 | 53 | 62 ± 14 | NA | 67 ± 11 | 59 ± 13 | 69 ± 10 | 49 ± 8 |
| Male BMI | 20.9 ± 0.9 | 25.0 ± 0.2 | 24.3 | 25.6 | NA | NA | 28.0 ± 4.8 | 26. ± 4.2 | NA | NA |
| Female N | 21 | 26 | 9 | 9 | 19 | NA | 26 | 29 | 21 | 16 |
| Female age | 59 ± 13 | 58 ± 11 | 74 | 57 | 63 ± 10 | NA | 70 ± 6 | 56 ± 15 | 70 ± 8 | 49 ± 6 |
| Female BMI | 19.9 ± 1.0 | 24.8 ± 0.4 | 23 | 29 | NA | NA | 25.5 ± 4.4 | 24.0 ± 4.5 | NA | NA |
| Stage 0/I | 8 | — | 5 | — | 10 | — | 13 | — | 9 | — |
| Stage II | 16 | — | 8 | — | 14 | — | 21 | — | 18 | — |
| Stage III | 15 | — | 8 | — | 12 | — | 25 | — | 11 | — |
| Stage IV | 1 | — | 2 | — | 8 | — | 7 | — | 2 | — |
| Unknown | 0 | — | 3 | — | 2 | — | 4 | — | 0 | — |

### Sample Extraction

Serum samples were stored at −80° C. until thawed for analysis, and were only thawed once. All extractions were performed on ice. Serum samples were prepared for FTICR-MS analysis by first sequentially extracting equal volumes of serum with 1% ammonium hydroxide and ethyl acetate (EtOAc) three times. Samples were centrifuged between extractions at 4° C. for 10 min at 3500 rpm, and the organic layer removed and transferred to a new tube (extract A). After the third EtOAc extraction, 0.33% formic acid was added, followed by two more EtOAc extractions. Following the final organic extraction, the remaining aqueous component was further extracted twice with water, and protein removed by precipitation with 3:1 acetonitrile (extract B). A 1:5 ratio of EtOAc to butanol (BuOH) was then evaporated under nitrogen to the original BuOH starting volume (extract C). All extracts were stored at −80° C. until FTICR-MS analysis.

### FTICR-MS Analysis

Sample extract (fraction C) was diluted ten-fold in methanol:0.1% (v/v) ammonium hydroxide (50:50, v/v) for negative ESI. For APCI, fraction A sample extracts were directly injected without diluting. All analyses were performed on a Bruker Daltonics APEX III FTICR-MS equipped with a 7.0 T actively shielded superconducting magnet (Bruker Daltonics, Billerica, Mass.). Samples were directly injected using ESI and APCI at a flow rate of 600 μL per hour. Ion transfer/detection parameters were optimized using a standard mix of serine, tetra-alanine, reserpine, Hewlett-Packard tuning mix and the adrenocorticotrophic hormone fragment 4-10. In addition, the instrument conditions were tuned to optimize ion intensity and broad-band accumulation over the mass range of 100-1000 amu according to the instrument manufacturer's recommendations. A mixture of the above mentioned standards was used to internally calibrate each sample spectrum for mass accuracy over the acquisition range of 100-1000 amu. FTICR data was analyzed using a linear least-squares regression line, mass axis values were calibrated such that each internal standard mass peak had a mass error of <1 PPM compared with its theoretical mass. Using XMASS™ software from Bruker Daltonics Inc., data file sizes of one megaword were acquired and zero-filled to two megawords. A SINm data transformation was performed prior to Fourier transform and magnitude calculations. The mass spectra from each analysis were integrated, creating a peak list that contained the accurate mass and absolute intensity of each peak. Compounds in the range of 100-1000 m/z were analyzed. In order to compare and summarize data across different ionization modes and polarities, all detected mass peaks were converted to their corresponding neutral masses, assuming hydrogen adduct formation. A self-generated two-dimensional (mass vs. sample intensity) array was then created using DISCO VAmetrics™ software (Phenomenome Discoveries Inc., Saskatoon, SK, Canada). The data from multiple files were integrated and this combined file was then processed to determine all of the unique masses. The average of each unique mass was determined, representing the y-axis. A column was created for each file that was originally selected to be analyzed, representing the x-axis. The intensity for each mass found in each of the files selected was then filled into its representative x,y coordinate. Coordinates that did not contain an intensity value were left blank. Each of the spectra was then peak-picked to obtain the mass and intensity of all metabolites detected. The data from all modes were then merged to create one data file per sample. The data from all 90 discovery serum samples were then merged and aligned to create a two-dimensional metabolite array in which each sample is represented by a column, each unique metabolite is represented by a single row, and each cell in the array corresponds to a metabolite intensity for a given sample. The array tables were then used for statistical analysis described in “statistical analyses”.

### Full-Scan Q-TOF and HPLC-Coupled Tandem Mass Spectrometry

Ethyl acetate extracts from five CRC and five normal samples were evaporated under nitrogen gas and reconstituted in 70 μL of isopropanol:methanol:formic acid (10:90:0.1). 10 μL of the reconstituted sample was subjected to HPLC (HP 1100 with Hypersil™ ODS 5 μm, 125×4 mm column, Agilent Technologies) for full scan and 30 μL for MS/MS at a flow rate of 1 ml/min. Eluate from the HPLC was analyzed using an ABI QSTAR® XL mass spectrometer fitted with an APCI source in negative mode. The scan type in full scan mode was time-of-flight (TOF) with an accumulation time of 1.0000 seconds, mass range between 50 and 1500 Da, and duration time of 55 min. Source parameters were as follows: Ion source gas 1 (GS1) 80; Ion source gas 2 (GS2) 10; Curtain gas (CUR) 30; Nebulizer Current (NC)-3.0; Temperature 400° C.; Declustering Potential (DP)-60; Focusing Potential (FP)-265; Declustering Potential 2 (DP2)-15. In MS/MS mode, scan type was product ion, accumulation time was 1.0000 seconds, scan range between 50 and 650 Da and duration time 55 min. All source parameters are the same as above, with collision energy (CE) of −35 V and collision gas (CID, nitrogen) of 5 psi. For MS3 work, the excitation energy was set at 180 V.

### Preliminary Isolation of CRC Biomarkers and NMR Analysis

For the thin layer chromatographic methods, all chemicals and media were purchased from Sigma-Aldrich Canada Ltd., Oakville, ON. All solvents were HPLC grade. Analytical TLC was carried out on pre-coated silica gel TLC aluminum sheets (EM science, Kieselgel 60 F254, 5×2 cm×0.2 mm). Compounds were visualized under UV light (254/366 nm) or placed in an iodine vapor tank and by dipping the plates in a 5% aqueous (w/v) phosphomolybdic acid solution containing 1% (w/v) ceric sulfate and 4% (v/v) H₂SO₄, followed by heating. NMR spectra were recorded on Bruker Avance spectrometers; for ¹H (500 MHz), δ values were referenced to CDCl₃ (CHCl₃ at 7.24 ppm) and for ¹³C NMR (125.8 MHz) referenced to CDCl₃ (77.23 ppm).

Ethyl acetate extracts of commercial serum (180 mL serum, 500 mg extract) was subjected to reverse phase flash column chromatography with a step gradient elution; acetonitrile-water 25:75 to 100% acetonitrile. The fractions collected were analyzed by LC/MS and MS/MS. The fractions containing the CRC biomarkers were pooled (12.5 mg). This procedure was repeated several times to obtain about 60 mg of CRC biomarker rich fraction. This combined sample was then subjected to FCC with a step gradient elution; hexane-chloroform-methanol and the fractions collected subjected to LC/MS and MS/MS analysis. The biomarker rich fraction labelled sample A (5.4 mg, about 65%) was analyzed by NMR. Sample A (3 mg) was then treated with excess ethereal diazomethane and kept overnight at room temperature. After the removal of solvent, the sample was analyzed by NMR.

### Triple-Quadrupole Multiple-Reaction-Monitoring (TQ-MRM) Methodology

Serum samples were extracted as described for non-targeted FTICR-MS analysis, with the addition of 10 ug/ml [¹³C₁]cholic acid to the serum prior to extraction (resulting in a final ethyl acetate concentration of [¹³C₁]cholic acid of 36 nM. The ethyl acetate organic fraction was used for the analysis of each sample. A series of [¹³C₁]cholic acid dilutions in ethyl acetate from Randox serum extracts was used to generate a standard curve ranging between 0.00022 ug/ml and 0.222 ug/ml. 100 uL of sample were injected by flow-injection analysis into the 4000QTRAP™ equipped with a TurboV™ source with an APCI probe. The carrier solvent was 90% methanol:10% ethyl acetate, with a flow rate of 360 uL/min into the APCI source. The source gas parameters were as follows: CUR: 10.0, CAD: 6, NC: −3.0, TEM: 400, GS1: 15, interface heater on. “Compound” settings were as follows: entrance potential (EP): −10, and collision cell exit potential (CXP): −20.0. The method is based on the multiple reaction monitoring (MRM) of one parent ion transition for each of the C28 molecules (445.3-383.4 Da, 447.4-385.4 Da, and 449.4-405.4 Da), and a single transition for the internal standard (408.3-343.4 Da). Each of the transitions was monitored for 250 ms for a total cycle time of 2.3 seconds. The total acquisition time per sample was approximately 1 min. All accepted analyses showed R2 correlation coefficients for the linear regression equation of >0.98. [¹³C₁]cholic acid equivalents for each of the three C28 molecules were calculated by determining the percent recovery of [¹³C₁]cholic acid in each sample by dividing the extrapolated concentration by 0.0148 ug/ml (36 nM, the theoretical amount present in the ethyl acetate extract of each sample). Metabolite concentrations represented as [¹³C₁]cholic acid equivalents were then extrapolated, normalized by dividing by the percent recovery, and multiplied by appropriate extraction dilution factors to yield a final serum concentration.

### Statistical Analysis

FTICR-MS accurate mass array alignments were performed using DISCO VAmetrics™ version 3.0 (Phenomenome Discoveries Inc., Saskatoon). Statistical analysis and graphs of FTICR-MS data was carried out using Microsoft™ Office Excel™ 2007, and distribution analysis of triple-quadrupole MRM data and was analyzed using JMP version 8.0.1. Meta Analysis (Fisher's Inverse Chi-square Method) was carried out using SAS 9.2 and R 2.9.0. Two-tailed unpaired Student's t-Tests were used for determination of significance between CRC and controls. P-values of less than 0.05 were considered significant. ROC curves were generated using the continuous data mode of JROCFIT (www.jrocfit.org).

### Results

### FTICR Metabolomic Profiling

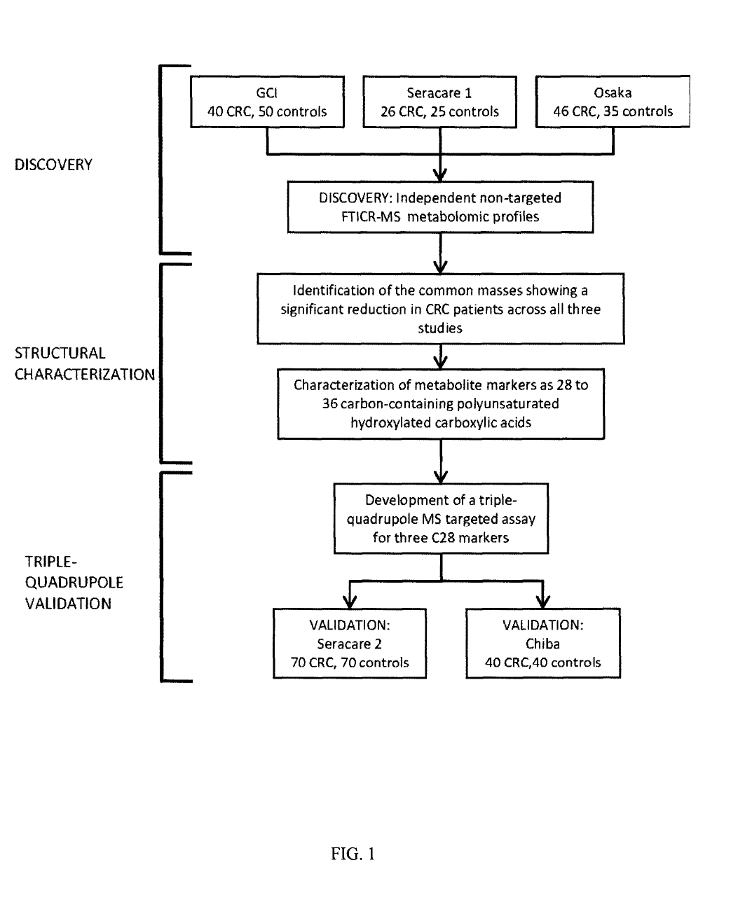

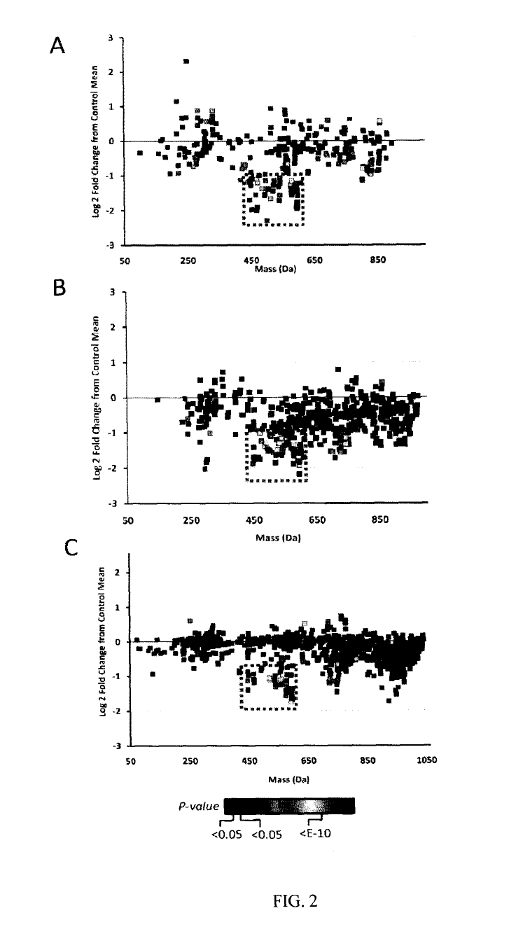

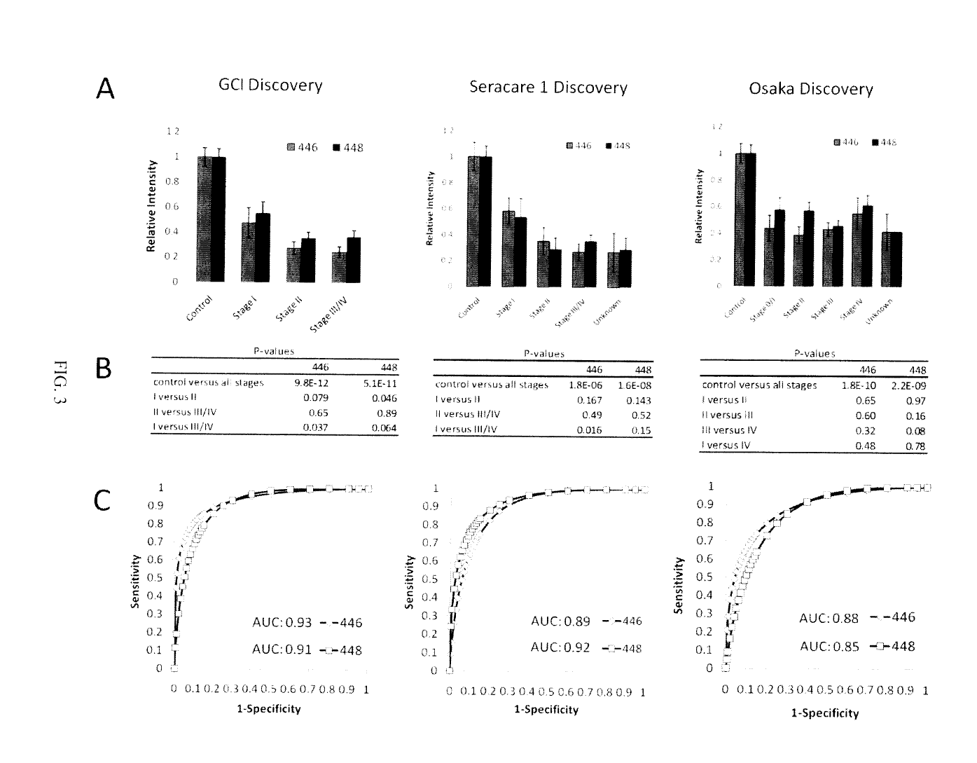

The experimental workflow for the described studies is summarized in FIG. 1. Non-targeted metabolomic profiles of sera from three independent populations of treatment-naive CRC patients and healthy controls (summarized in Table 1) were generated over a 24-month period (i.e., each study was separated by approximately 12 months). The first study comprised 40 CRC patients and 50 control subjects acquired from Genomics Collaborative, Inc (GCI); the second study comprised 26 CRC subjects and 25 controls acquired from Seracare Lifesciences Inc, and the third study included 46 CRC and 35 controls prospectively collected in Osaka, Japan (Monden et al). In all cases, serum metabolites were captured through a liquid extraction process (see methods above), followed by direct infusion of the extracts using negative electrospray ionization (nESI) and negative atmospheric pressure chemical ionization (nAPCI) on an FTICR mass spectrometer. The resulting spectral data of all the subjects for each study was aligned within 1 PPM mass accuracy, background peaks were subtracted, and a two-dimensional array table comprising the intensities each of the sample-specific spectral peaks was created using custom informatics software (see methods above). Metabolic differences between CRC patient and control profiles for the three independent studies were visualized by plotting the control mean-normalized log ratio peak intensities across the detected mass range as shown in FIGS. 2A to 2C. In each independent study, a region of spectra between approximately 440 and 600 Da showed peaks consistently reduced in intensity in CRC patients relative to controls (green, yellow, orange and red points in FIG. 2). On average, this cluster of masses showed between 50% and 75% reduction in CRC patient serum compared to controls, with p-values of 1×10⁻⁵ or lower in each study.

The overlap between each of the discovery studies was further investigated by ranking the top 50 masses based upon p-value from each study and comparing them with masses showing a significant difference (p<0.05 between CRC and controls) in the other studies as shown in Table 2. For example, 46 of the top 50 metabolites (92%) with the lowest p-values in the GCI discovery set were also found to be significantly different in the Seracare 1 dataset, while 31 out of the 50 GCI masses were also detected with p<0.05 in the Osaka dataset. Likewise, the top 50 metabolites in the Osaka study showed 88% and 94% redundancy with metabolites showing p<0.05 in the GCI and Seracare 1 studies, respectively. These results indicated a very high degree of commonality among significantly differentiated masses across the three studies, and in fact, 63% of the top 50 masses in each study were also present within the top 50 of at least one of the other two studies (See Table 2.1). Of the top 50 rank-ordered masses, only those identified in more than one study were found to exist within the 440 to 600 Da mass range highlighted above, and there was not a single peak detected outside this region which was significantly different between CRCs and controls in any two of the studies. Filtering for metabolic differences detected exclusively in all three studies (as well as removal of C13 isotopic peaks and redundant masses detected in both ESI and APCI), resulted in 13 masses representing individual ¹²C metabolites as shown in Table 3. The masses exhibited similar expression profile across patient samples within each study, suggesting that they may be related (as assessed by Pearson correlation coefficients; not shown). Bar charts of the two smallest molecular weight molecules with the nominal masses of 446 and 448 are shown in FIG. 3. Little to no correlation was observed between the reduction of the metabolites and disease stage (FIGS. 3A and 3B), and receiver-operator characteristic curve analysis resulted in an average area under-the-curve (AUC) of 0.91±0.03 (FIG. 3C; individual AUCs shown) across all three studies for all stages combined.

Computational assignments of reasonable molecular formulas were then carried out for the 13 masses identified above. The assignments were based on a series of mathematical and chemometric rules as described previously²³, which are reliant on high mass accuracy for precise prediction. The algorithm computes the number of carbons, hydrogens, oxygens, and other elements, based on their exact mass, which can be assigned to a detected accurate mass within defined constraints. Logical putative molecular formulas were computed for masses in Table 3, resulting in elemental compositions containing either 28, 30, 32 or 36 carbons and four to six oxygen. Several classes of metabolites, including various forms of fat soluble vitamins, steroids and fatty acids theoretically fit these elemental compositions. We used this information in the subsequent section to select appropriate molecules for structural comparison studies. Collectively, the results indicated a consistent 50% to 75% reduction of organically soluble oxygenated metabolites ranging between 28 and 36 carbons in length, in the serum of CRC patients compared to controls.

TABLE 2
Percent overlap between top 50 most discriminating masses (based on
student's t-test) of each discovery project and masses showing
p < 0.05 in the remaining cohorts.

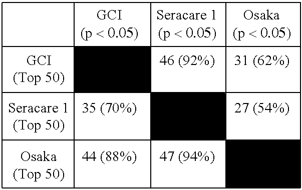

TABLE 2.1
Top 50 discriminating masses (based on student's t-test) of each discovery project. Masses shaded grey were detected in
the top 50 in two of the three studies. Indicated are the detected accurate mass, the computationally predicted molecular formula
(for masses shaded in grey), the mass difference between the detected mass and mass of the predicted molecular formula in part
per million (PPM), the mode of analysis (electrospray ionization, ESI atmospheric pressure chemical ionization, APCI), the
p-value (based on an unpaired student's t-test) between the average peak intensity of control subjects versus CRC patients,
and the average peak intensity ratio between CRC patients and controls.
GO

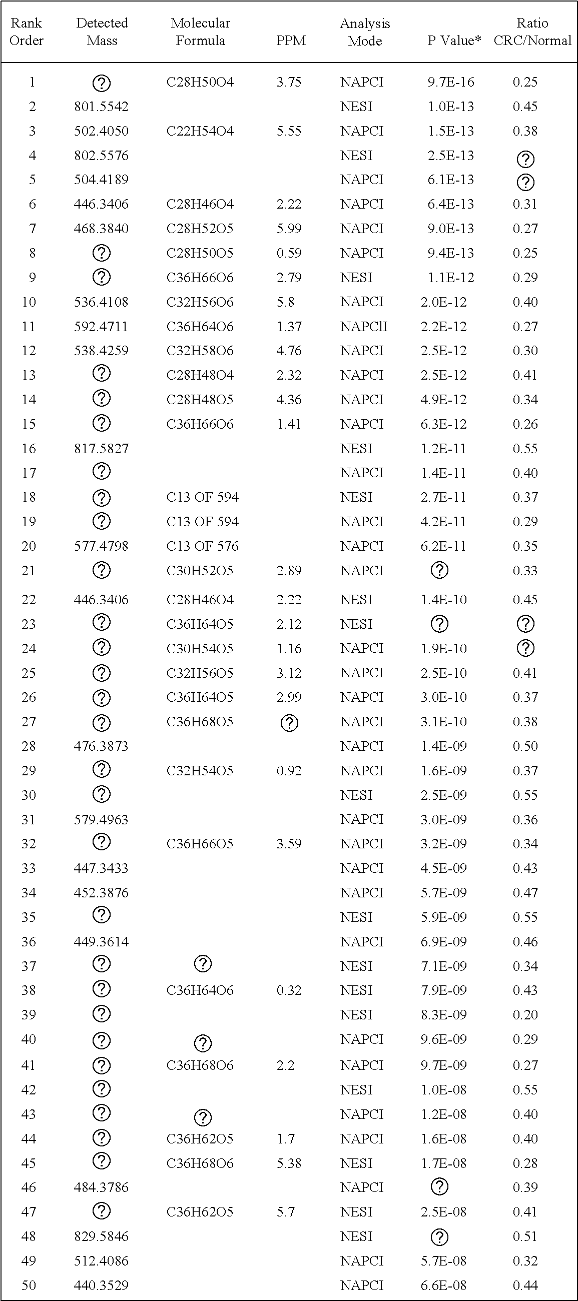

SERACARE 1

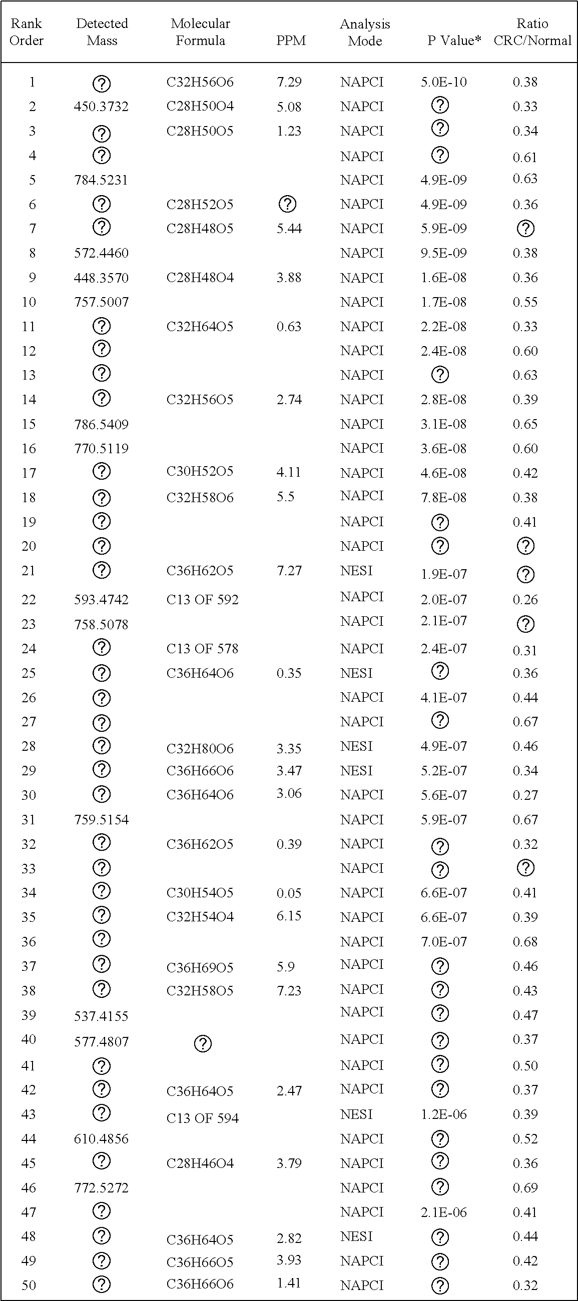

OSAKA

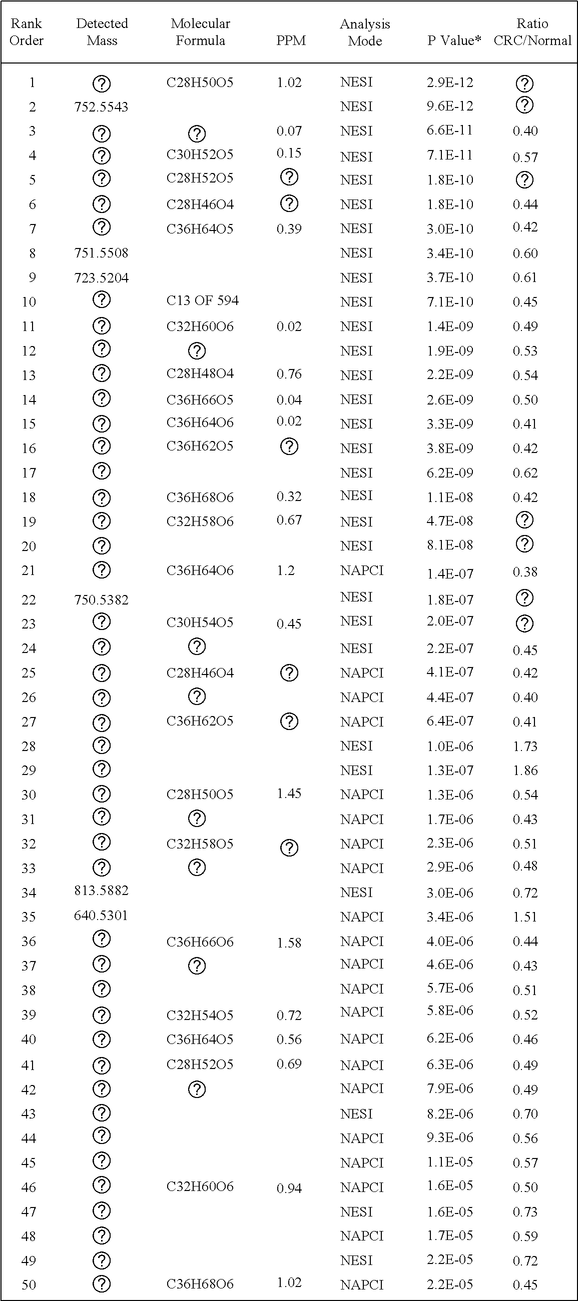

TABLE 3
List of 13 masses detected among the top 50 masses
inclusive to all three discovery projects.

| | | | | | | |
|---|---|---|---|---|---|---|
| | | | | | | Ratio |
| Rank | Detected | Molecular | | Analysis | | CRC/ |
| Order | Mass | Formula | PPM | Mode | P Value* | Normal |

GCI

| | | | | | | |
|---|---|---|---|---|---|---|
| 6 | 446.3406 | C28H46O4 | 2.22 | NAPCI | 6.4E−13 | 0.31 |
| 13 | 448.3563 | C28H48O4 | 2.32 | NAPCI | 2.5E−12 | 0.41 |
| 8 | 466.3661 | C28H50O5 | 0.59 | NAPCI | 9.4E−13 | 0.25 |
| 7 | 468.3840 | C28H52O5 | 5.39 | NAPCI | 9.0E−13 | 0.27 |
| 21 | 492.3829 | C30H52O5 | 2.89 | NAPCI | 8.5E−11 | 0.33 |
| 24 | 494.3977 | C30H54O5 | 1.16 | NAPCI | 1.9E−10 | 0.35 |
| 29 | 518.3976 | C32H54O5 | 0.92 | NAPCI | 1.6E−09 | 0.37 |
| 12 | 538.4259 | C32H58O6 | 4.76 | NAPCI | 2.5E−12 | 0.30 |
| 44 | 574.4607 | C36H62O5 | 1.7 | NAPCI | 1.6E−08 | 0.40 |
| 26 | 576.4771 | C36H64O5 | 2.99 | NAPCI | 3.0E−10 | 0.37 |
| 32 | 578.4931 | C36H66O5 | 3.59 | NAPCI | 3.2E−09 | 0.34 |
| 11 | 592.4711 | C36H64O6 | 1.37 | NAPCI | 2.2E−12 | 0.27 |
| 15 | 594.4851 | C36H66O6 | 1.41 | NAPCI | 6.3E−12 | 0.26 |

SERACARE 1

| | | | | | | |
|---|---|---|---|---|---|---|
| 45 | 446.3413 | C28H46O4 | 3.79 | NAPCI | 1.8E−06 | 0.36 |
| 9 | 448.3570 | C28H48O4 | 3.88 | NAPCI | 1.6E−08 | 0.36 |
| 3 | 466.3664 | C28H50O5 | 1.23 | NAPCI | 8.5E−10 | 0.34 |
| 6 | 468.3847 | C28H52O5 | 6.89 | NAPCI | 4.9E−09 | 0.36 |
| 17 | 492.3835 | C30H52O5 | 4.11 | NAPCI | 4.6E−08 | 0.42 |
| 34 | 494.3971 | C30H54O5 | 0.05 | NAPCI | 6.6E−07 | 0.41 |
| 11 | 518.3968 | C32H54O5 | 0.63 | NAPCI | 2.2E−08 | 0.33 |
| 18 | 538.4263 | C32H58O6 | 5.5 | NAPCI | 7.8E−08 | 0.38 |
| 32 | 574.4595 | C36H62O5 | 0.39 | NAPCI | 6.1E−07 | 0.32 |
| 42 | 576.4768 | C36H64O5 | 2.47 | NAPCI | 1.0E−06 | 0.37 |
| 49 | 578.4933 | C36H66O5 | 3.93 | NAPCI | 3.2E−06 | 0.42 |
| 30 | 592.4721 | C36H64O6 | 3.06 | NAPCI | 5.6E−07 | 0.27 |
| 50 | 594.4851 | C36H66O6 | 1.41 | NAPCI | 3.7E−06 | 0.32 |

OSAKA

| | | | | | | |
|---|---|---|---|---|---|---|
| 6 | 446.3400 | C28H46O4 | 0.87 | NESI | 1.8E−10 | 0.44 |
| 13 | 448.3556 | C28H48O4 | 0.76 | NESI | 2.2E−09 | 0.54 |
| 1 | 466.3663 | C28H50O5 | 1.02 | NESI | 2.9E−12 | 0.50 |
| 5 | 468.3815 | C28H52O5 | 0.05 | NESI | 1.8E−10 | 0.49 |
| 4 | 492.3814 | C30H52O5 | 0.15 | NESI | 7.1E−11 | 0.57 |
| 23 | 494.3969 | C30H54O5 | 0.45 | NESI | 2.0E−07 | 0.62 |
| 39 | 518.3975 | C32H54O5 | 0.72 | NAPCI | 5.8E−06 | 0.52 |
| 19 | 538.4237 | C32H58O6 | 0.67 | NESI | 4.7E−08 | 0.58 |
| 16 | 574.4600 | C36H62O5 | 0.48 | NESI | 3.8E−09 | 0.42 |
| 7 | 576.4756 | C36H64O5 | 0.39 | NESI | 3.0E−10 | 0.42 |
| 14 | 578.4910 | C36H66O5 | 0.04 | NESI | 2.6E−09 | 0.50 |
| 15 | 592.4703 | C36H64O6 | 0.02 | NESI | 3.3E−09 | 0.41 |
| 3 | 594.4859 | C36H66O6 | 0.07 | NESI | 8.8E−11 | 0.40 |

Indicated are the rank order based on p-value, detected accurate mass, the computationally predicted molecular formula, the mass difference between the detected mass and mass of the predicted molecular formula in part per million (PPM), the mode of analysis (electrospray ionization, ESI; atmospheric pressure chemical ionization, APCI), the p-value (based on an unpaired student's t-test) between the average peak intensity of control subjects versus CRC patients, and the average peak intensity ratio between CRC patients and controls.

### HPLC-Coupled Tandem Mass Spectrometry

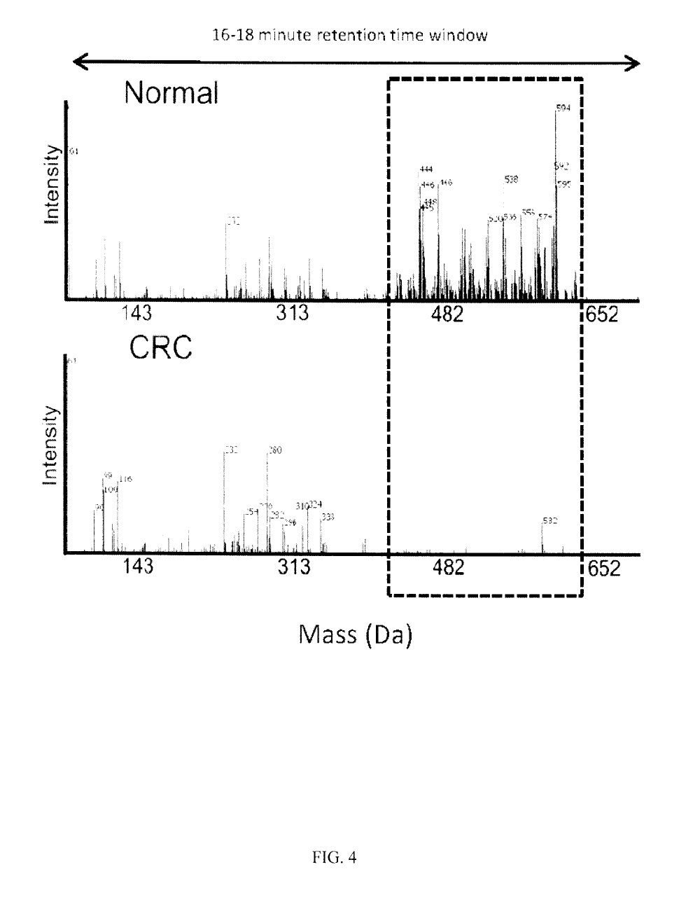

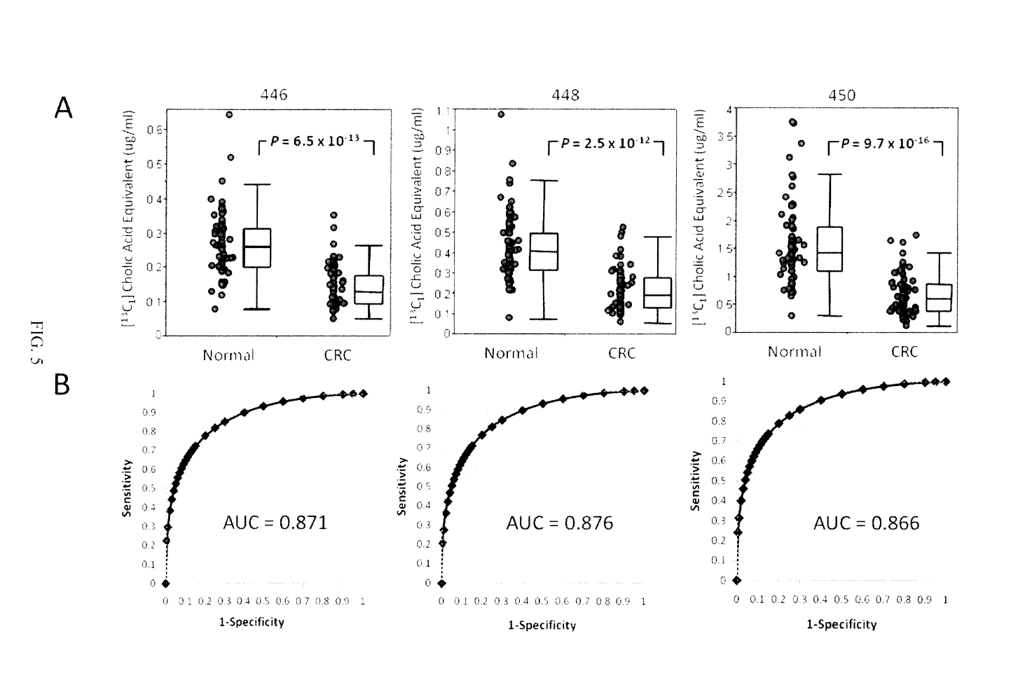

Selected ethyl acetate extracts of serum from the GCI cohort used in the FTICR-MS work described above were re-analyzed using HPLC coupled to a quadrupole time-of-flight (Q-TOF) mass spectrometer in full-scan APCI negative ion mode. Consistent with the FTICR-MS results, a cluster of peaks between approximately 440 and 600 Da at a retention time of between 16 and 18 minutes following reverse-phase HPLC was detected in asymptomatic control sera, but absent from CRC patient serum (FIG. 4). Molecular ions from all six C28 biomarkers (m/z 446, m/z 448, m/z 450, m/z 464, m/z 466 and m/z 468) as well as many of the remaining C32 and C36 markers were detectable within the normal serum cluster. Extracted masses up to 400 Da within the 16-18 minute retention time showed similar peak intensities in both populations (FIG. 4, region to the right of the box), as did extracted mass spectra at other retention times (not shown), reinforcing the specificity of this depleted metabolic region for CRC patient serum.

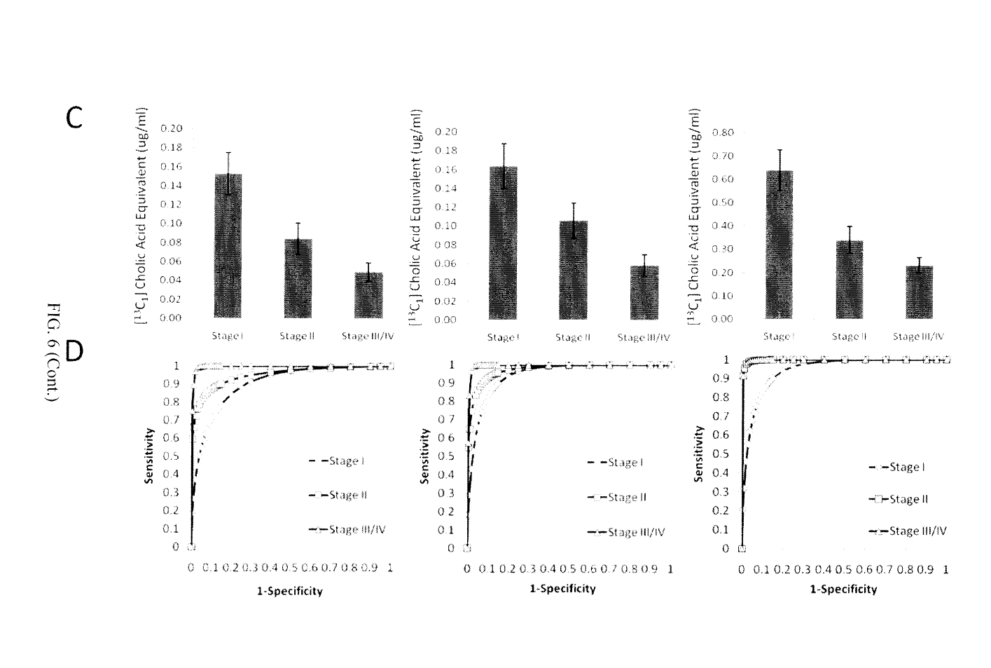

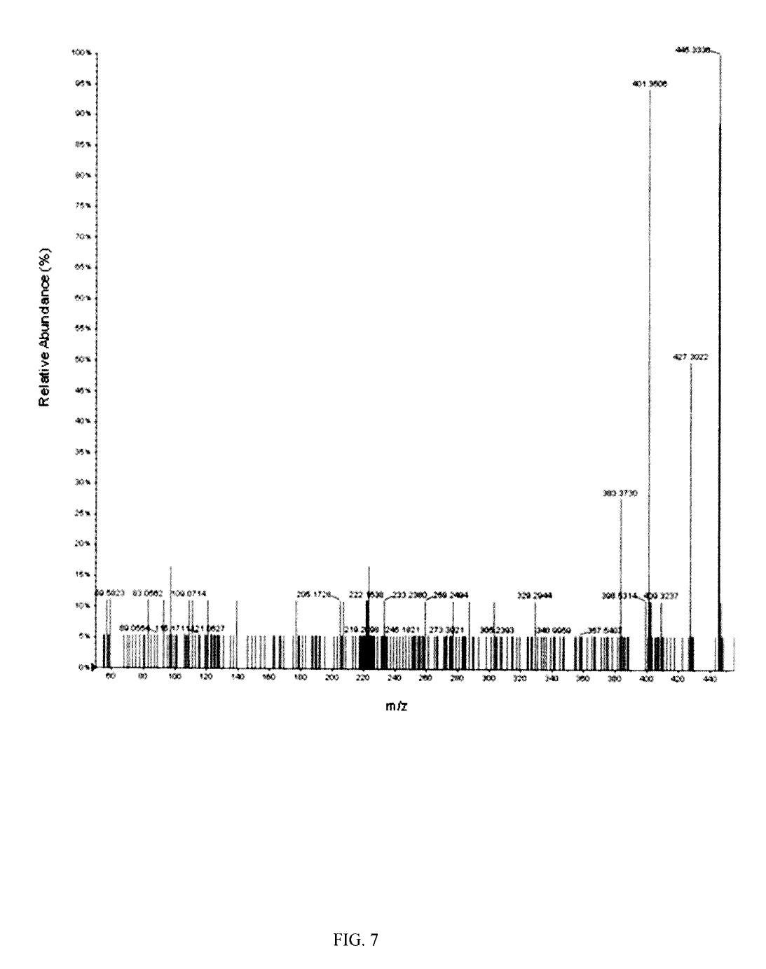

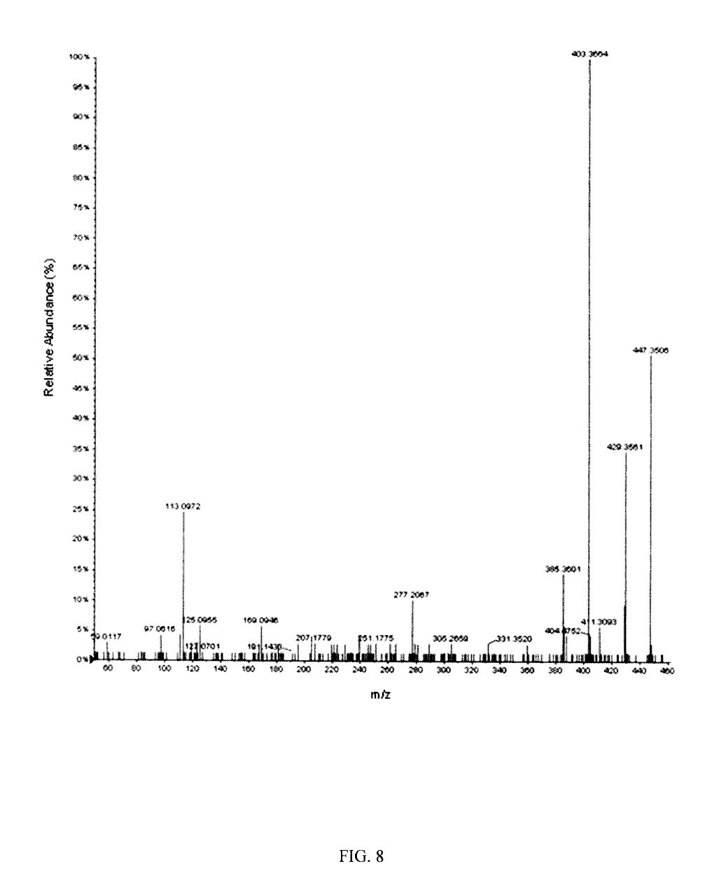

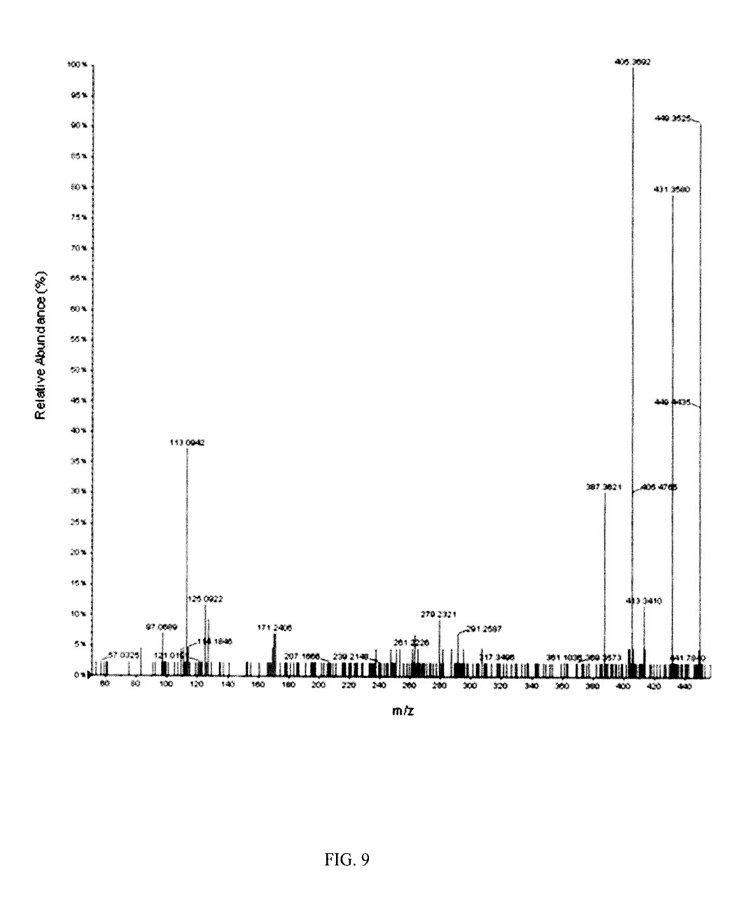

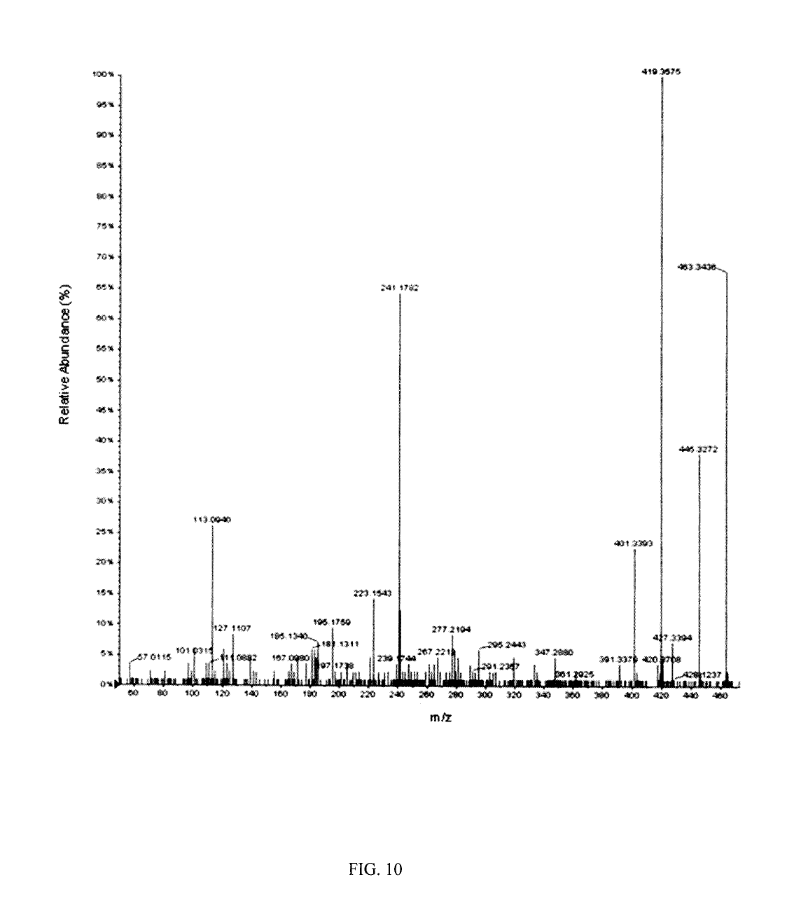

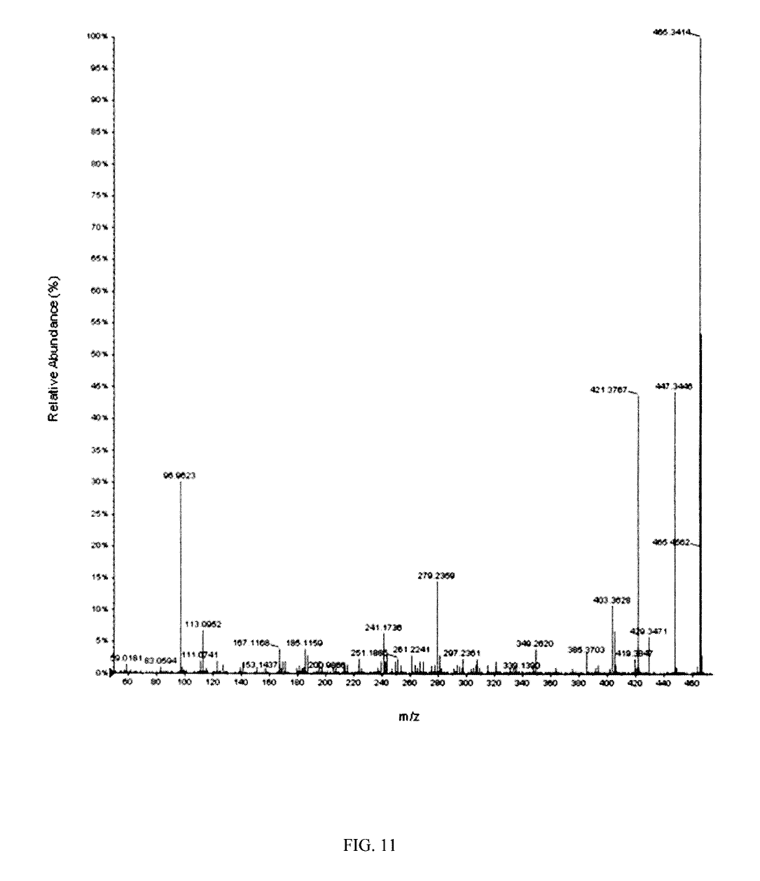

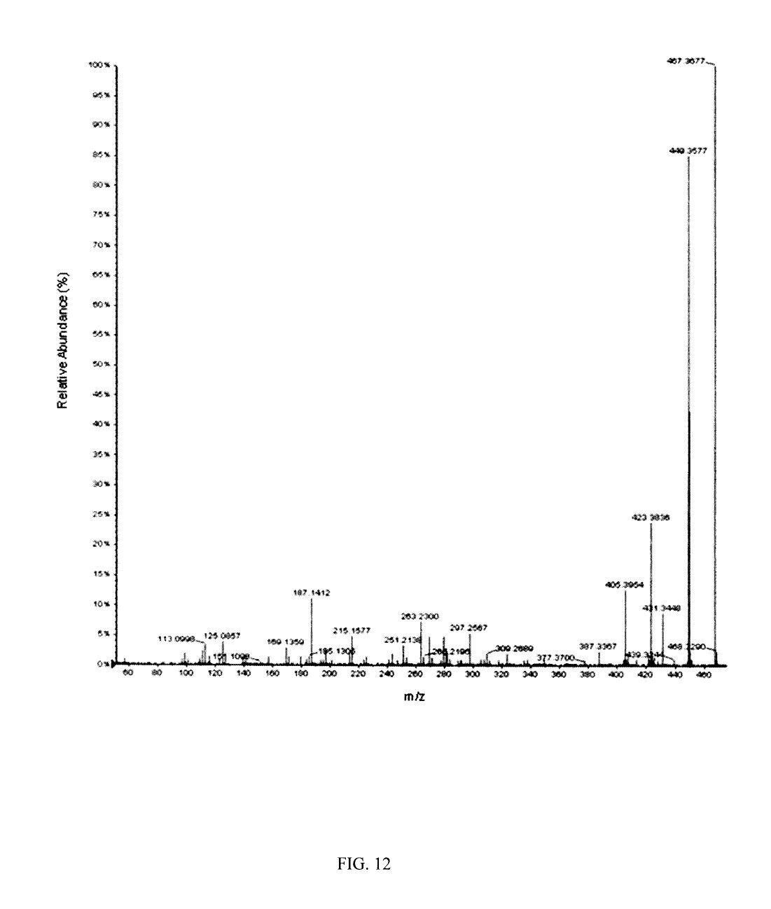

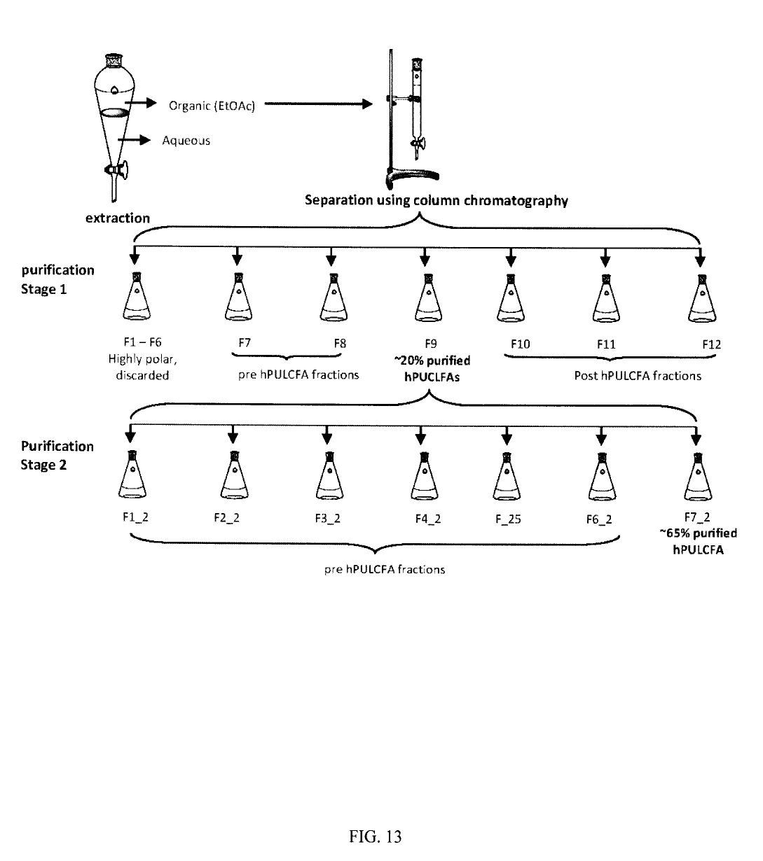

Tandem mass spectrometric fragmentation fingerprints were next generated for the six C28 biomarkers (Table 4, see also FIGS. 7 to 12) and for the higher C32 and C36 biomarkers (See Table 4.1). The MS/MS and MS3 fragmentation data of the six C28 biomarkers were dominated by peaks resulting from losses of H₂O (m/z 427, 429, 431, 445, 447 and 449), losses of 2 molecules of H₂O (m/z 409, 411, 413, 427, 429, 431), losses of CO₂ (m/z 401, 403, 405, 419, 421, 423) and losses of CO₂ and H₂O (m/z 383, 385, 387, 401, 403, 405), indicating the presence of carboxylic acid functionality and two or more hydroxyl groups. Based upon the molecular formulae, the organic properties of the molecules and the tandem MS data, we hypothesized that the metabolites may be derivatives or analogs of one or more possible classes of molecules including fat soluble vitamins such as retinol and retinoic acid (vitamin A), calciferols (vitamin D), tocopherols (vitamin E), phylloquinones (vitamin K), steroids or bile acids, or long chain polyunsaturated hydroxy fatty acids. Tandem mass spectrometric fragmentation fingerprints were therefore generated for standards 5S,6S-(7E,9E,11Z,14Z)-dihydroxyeicosatetraenoic acid (1), 15S-Hydroxy-(5Z,8Z,11Z,13E)-eicosatetraenoic acid (2) and 8R-Hydroxy-(5Z,9E,11Z,14Z)-eicosatetraenoic acid (3), α-tocopherol (4) γ-tocopherol (5), 13-(6-hydroxy-2,7,8-trimethylchroman-2-yl)-2,6,10-trimethyltridecanoic acid (6), 16-(4,5-dimethyl-3,6-dioxo cyclohexa-1,4-dienyl)-2,6,10,14-tetramethylhexadecanoic acid (7), 6-hydroxy-2,7-dimethyl-2-(4,8,12-trimethyltridecyl)chroman-8-carbaldehyde (8), 6-hydroxy-2,7-dimethyl-2-(4,8,12-trimethyltridecyl)chroman-8-carboxylic acid (9), calciferol (10), cholecalciferol (11), ergosterol (12), phylloquinone (13), retinol (14) and 3β,7α-dihydroxy-5-cholestenoic acid (15) (Table 5). The resulting MS/MS data for vitamins A, D, E, K as well as the steroidal molecules (4-15) showed no similarity to any of the metabolomic biomarkers; for vitamin E type molecules, all had diagnostic fragments characteristic of their chroman rings (m/z 163, 149, 149, 149, 163 and 179 for 4, 5, 6, 7, 8 and 9 respectively), for vitamin D and analogs, diagnostic fragments formed as a result of the loss of the side chain (m/z 271, 273 and 253, for 10, 11 and 13 respectively), for phylloquinone (13), the diagnostic fragment m/z 187 for the quinone ring system was prominent, for vitamin A (14), the fragment m/z 269 (M+H−H₂O) loses the cyclohexyl ring moiety to form a diagnostic m/z 145 for retinol, and for 3β,7α-dihydroxy-5-cholestenoic acid (15) the diagnostic retro diels alder fragment at m/z 277 was observed. In addition to this, other carboxylic acid standards with a pregnane ring system as in 15, (for example, chenodeoxycholic acid and cholic acid) do not show losses of CO₂ upon MS/MS fragmentation (not shown). MS/MS fragmentation data of hydroxy fatty acid standards 1, 2 and 3 (Table 5), however, showed peripheral cut ions similar to those produced by MS/MS of the CRC biomarkers, and consistent with what has been described by others for various hydroxylated long-chain fatty acids²⁹⁻³³. For example, marker m/z 446 showed peripheral cut ions 427 [M−H−H₂O]—, 401 [M−H−CO₂]—, 409 [M−H−2H₂O]—, 383 [M H−CO₂−H₂O]— and 365 [M−H−CO₂−2H₂O]— and chain cut ions, 223, 205, 277 as well as others (see Table 5 and FIG. 7). Similar ions were obtained for the other C28,C32 and C36 metabolites (Table 4, See Table 4.1). Collectively, these deductions suggested that the metabolomic markers were not likely analogs of vitamins A, D, E K and steroids, but rather long-chain fatty acid-type molecules containing several unsaturations, and hydroxy groups. We collectively refer to these metabolites as hydroxy polyunsaturated ultra long-chain fatty acids (hPULCFAs; where the term “ultra” has been used to refer to C30 and longer chain fatty acids³⁴).

TABLE 4
Tandem-MS analysis of selected 28-carbon containing masses.

| | | | Peripheral Cut ions (%) | | | | | | |
|---|---|---|---|---|---|---|---|---|---|
| CRC | | Chain | | | | Loss of | *Loss of | | |
| Biomarker | | Cut ions | Loss of | Loss of | Loss of | CO₂ and | CO₂ and | *Loss of | Secondary |
| M | [M − H]⁻ (%) | (%) | H₂O | 2H₂O | CO₂ | H₂O | 2H₂O | 3H₂O | Daughter ions (%) |
| 446 | 445 (100%) | 223 (18%), | 427 (50%) | 409 (8%) | 401 (95%) | 383 (28%) | 365 | — | 357 (5%), 329 (11%), |
| | | 222 (11%), | | | | | | | 261 (3%), 241 (3%), |
| | | 207 (3%), | | | | | | | 233 (5%), 207 (11), |
| | | 205 (11%), | | | | | | | 177 (11%), 123 |
| | | 113 (5%). | | | | | | | (5%), 109 (11%), 97 |
| | | | | | | | | | (16%), 83 (11%), 59 |
| | | | | | | | | | (11%). |
| 448 | 447 (52%) | 277 (11%), | 429 (35%) | 411 (6%) | 403 (100%) | 385 (15%) | 367 | — | 331 (3%), 305 (3%), |
| | | 239 (5%), | | | | | | | 359 (2%), 289 (3%), |
| | | 207 (3%), | | | | | | | 245 (3%), 125 (6%), |
| | | 169 (6%), | | | | | | | 123 (3%), 121 (3%), |
| | | 113 (25%). | | | | | | | 111 (5%), 97 (5%), |
| | | | | | | | | | 59 (3%). |
| 450 | 449 (92%) | 171 (7%), | 431 (80%) | 413 (13%) | 405 (100%) | 387 (32%) | 369 | — | 307 (5%), 291 (7%), |
| | | 127 (9%), | | | | | | | 295 (5%), 281 (5%), |
| | | 125 (12%), | | | | | | | 279 (9%), 263 (7%), |
| | | 113 (38%). | | | | | | | 261 (5%), 169 (5%), |
| | | | | | | | | | 111 (5%), 97 (8%), |
| | | | | | | | | | 83 (5%), 59 (1%). |
| 464 | 463 (70%) | 277 (10%), | 445 (46%) | 427 (6%) | 419 (100%) | 401 (24%) | 383 (2%) | 409 | 347 (5%), 319 (5%), |
| | | 241 (68%), | | | | | | | 295 (6%), 281 (5%), |
| | | 223 (15%) | | | | | | | 279 (5%), 267 (5%), |
| | | 185 (8%), | | | | | | | 249 (6%), 195 (10%), |
| | | 167 (4%), | | | | | | | 141 (1%), 127 (9%), |
| | | 113 (28%). | | | | | | | 121 (6%), 101 (6%), |
| | | | | | | | | | 97 (4%), 83 (2%), 59 |
| | | | | | | | | | (2%). |
| 466 | 465 (100%) | 241 (7%), | 447 (45%) | 429 (8%) | 421 (45%) | 403 (20%) | 385 (4%) | 411 | 349 (4%), 321 (2%), |
| | | 223 (3%), | | | | | | | 297 (3%), 281 (3%), |
| | | 215 (2%), | | | | | | | 279 (15%), 261 (3%), |
| | | 185 (4%), | | | | | | | 251 (3%), 195 (2%), |
| | | 167 (4%), | | | | | | | 141 (2%), 123 (4%), |
| | | 113 (7%). | | | | | | | 113 (5%), 101 (3%), |
| | | | | | | | | | 97 (32%), 83 (2%), |
| | | | | | | | | | 59 (2%). |
| 468 | 467 (100%) | 187 (12%), | 449 (84%) | 431 (10%) | 423 (25%) | 405 (13%) | 387 (3%) | 413 | 349 (1%), 323 (2%), |
| | | 169 (3%), | | | | | | | 309 (2%), 297 (6%), |
| | | 141 (2%) | | | | | | | 281 (3%), 279 (5%), |
| | | 113 (4%). | | | | | | | 269 (5%), 263 (8%), |
| | | | | | | | | | 251 (4%), 243 (2%), |
| | | | | | | | | | 215 (4%), 213 (3%), |
| | | | | | | | | | 197 (3%), 125 (4%), |
| | | | | | | | | | 111 (3%), 98 (2%), |
| | | | | | | | | | 57 (1%). |

TABLE 4.1
MSMS of hPULCFAs.

| Mass | Parent and daughter ions (%) |
|---|---|
| 452 | |
| 476 | 475 (100%), 457 (65%), 431 (40%), 413 (10%), 255 (5%) |
| 492 | 491 (100%), 473 (40%), 455 (5%), 447 (35%), 375 (5%), 319 (10%), 267 (10%), 241 (20%) |
| 494 | 493 (100%), 475 (50%), 449 (30%), 431 (10%), 297 (5%), 215 (5%) |
| 496 | 495 (100%), 477 (50%), 459 (10%), 451 (20%), 433 (10%), 215 (5%) |
| 502 | 501 (100%), 483 (40%), 465 (5%), 457 (20%), 439 (20%), 281 (5%) |
| 504 | 503 (100%), 485 (30%), 459 (40%), 441 (20%), 113 (10%) |
| 512 | 511 (60%), 493 (10%), 451 (10%), 315 (100%) |
| 518 | 517 (100%), 499 (20%), 481 (5%), 473 (10%), 401 (5%) |
| 520 | 519 (100%), 501 (30%), 483 (5%), 475 (10%) |
| 522 | 521 (100%), 503 (25%), 485 (5%), 477 (10%), 459 (10%) |
| 536 | 535 (100%), 517 (30%), 499 (6%), 491(6%), 473 (25%), 455 (3%), 254 (3%), 239 (3%) |
| 538 | 537 (100%), 519 (20%), 501 (1%), 493 (2%), 475 (2%) |
| 540 | 539 (30%), 521 (3%), 503 (1%), 495 (3%), 477 (1%), 315 (100%), 223 (3%), 179 (3%). |
| 558 | 557 (85%), 539 (25%), 513 (15%), 489 (30%), 454 (15%), 245 (100%), 203 (40%) |
| 560 | 559 (100%), 541 (20%), 515 (40%), 497 (5%), 485 (10%), 201 (20%), 113 (20%) |
| 562 | 561 (100%), 543 (5%), 517 (5%), 501 (3%), 501 (1%), 499 (2%), 113 (50%), 85 (20%), 75 (40%) |
| 574 | 575 (100%), 555 (5%), 537 (2%), 529 (1%), 505 (2%), 415 (5%), 401 (1%) |
| 576 | 575 (100%), 557 (20%), 539 910%), 531 (20%), 513 (15%), 495 (5%), 416 (5%), 259 (5%) |
| 578 | 577 (20%), 555 (3%), 533 (5%), 401 (5%), 175 (10%), 113 (100%), 103 (15%), 85 (20%) |
| 580 | 579 (100%), 561 (45%), 535 (30%), 517 (15%), 479 (10%), 417 (10%), 315 (10%), 245 (50%), 175 (20%), 113 (40%), 85 (15%) |
| 590 | 589 (100%), 571 (20%), 545 (10%), 527 (15%), 201 (20%), 113 (15%) |
| 592 | 591 (100%), 547 (5%), 555 (20%), 529 (10%), 415 (60%), 400 (10%), 113 (40%) |
| 594 | 593 (100%), 557 (5%), 549 ( [symbol] %), 534 ( [symbol] %), 5 [symbol] ( [symbol] %), 435 ( [symbol] %) |
| 596 | 595 (10%), 576 (10%), 551 (10%), 437 (10%), 423 (10%), 267 (15%), 2415 (15%), 171 (10%) |

[symbol]

indicates data missing or illegible when filed

TABLE 5
Tandem mass spectrometric results of various standards.

| Standard | | 1 | 2 | 3 | 4 | 5 | 6 | 7 | 8 |
|---|---|---|---|---|---|---|---|---|---|
| [M − H]− (%) | | 335 (45%) | 319 (100%) | 319 (100%) | 429 (14%) | 415 (28%) | 445 (90%) | 445 (68%) | 429 (100%) |
| *Chain cut ions (%) | | 219 (15%) | 219 (80%) | 203 (3%) | | | | | |
| | | 201 (1%) | 203 (15%) | 163 (25%) | | | | | |
| | | 115 (100%) | 175 (55%) | 155 (60%) | | | | | |
| | | | 113 (20%) | 127 (10%) | | | | | |
| | | | | 111 (5%) | | | | | |
| *Peripheral | Loss of H₂O | 317 (16%) | 301 (60%) | 301 (55%) | — | — | 427 (50%) | — | 401 (30%) |
| cut ions (%) | Loss of 2H₂O | 299 | — | — | — | — | — | — | — |
| | Loss of CO₂ | 291 (2%) | 275 (20%) | 275 (4%) | — | — | 401 (1%) | 401 (35%) | — |
| | Loss of CO₂ | 273 (6%) | 257 (70%) | 257 (80%) | — | — | — | — | — |
| | and H₂O | | | | | | | | |
| | *Loss of CO₂ | — | — | — | — | — | — | — | — |
| | and 2H₂O | | | | | | | | |
| | *Loss of 3H₂O | — | — | — | — | — | — | — | — |

| Secondary daughter ions (%) | | 189 (1%) | 167 (5%) | 291 (1%) | 414 (5%) | 400 (78%) | 295 (70%) | 386 (52%) | 163 (100%) |
|---|---|---|---|---|---|---|---|---|---|
| | | 163 (1%) | 149 (2%) | 171 (1%) | 163 (100%) | 175 (13%) | 149 (90%) | 179 (60%) | 135 (20%) |
| | | 145 (2%) | 121 (20%) | 107 (1%) | 135 (8%) | 149 (100%) | 136 (100%) | 135 (100%) | 218 (5%) |
| | | 99 (1%) | 99 (1%) | 59 (1%) | | 121 (90%) | 121 (20%) | 107 (25%) | 123 (5%) |
| | | 95 (1%) | 59 (1%) | | | | | | |
| | | 71 (1%) | | | | | | | |
| | | 59 (1%) | | | | | | | |
| | Standard | | 9 | 10 | 11 | 12 | 13 | 14 | 15 |
| | [M — H]− (%) | | 445 (64%) | 397 (6%) | 385 (2%) | 397 (5%) | 451 (28%) | 287 (1%) | 431 (100%) |
| | *Chain cut ions (%) | | | | | | | | |
| | *Peripheral | Loss of H₂O | 401 (28%) | 379 (10%) | 367 (18%) | 379 (5%) | — | 269 (1%) | 413 (1%) |
| | cut ions (%) | Loss of 2H₂O | — | — | — | — | — | — | — |
| | | Loss of CO₂ | — | — | — | — | — | — | — |
| | | Loss of CO₂ | — | — | — | — | — | — | — |
| | | and H₂O | | | | | | | |
| | | *Loss of CO₂ | — | — | — | — | — | — | — |
| | | and 2H₂O | | | | | | | |
| | | *Loss of 3H₂O | — | — | — | — | — | — | — |

| | | | | | | | |
|---|---|---|---|---|---|---|---|
| Secondary daughter ions (%) | 386 (43%) | 309 (5%) | 273 (3%) | 295 (5%) | 436 (10%) | 187 (5%) | 399 (100%) |

| | | | | | | |
|---|---|---|---|---|---|---|
| 179 (50%) | 213 (15%) | 259 (25%) | 253 (6%) | 241 (21%) | 173 (10%) | 393 (40%) |
| 166 (20%) | 201 (30%) | 255 (15%) | 211 (12%) | 227 (62%) | 159 (18%) | 373 (40%) |
| 135 (100%) | 173 (37%) | 213 (20%) | 159 (20%) | 223 (48%) | 145 (28%) | 355 (40%) |
| 122 (25%) | 159 (35%) | 173 (47%) | 161 (15%) | 213 (55%) | 119 (15%) | 337 (20%) |
| 107 (25%) | 107 (90%) | 161 (52%) | 147 (20%) | 199 (57%) | 105 (20%) | 223 (40%) |
| | 81 (68%) | 159 (75%) | 107 (25%) | 187 (100%) | 95 (36%) | 85 (40%) |
| | 69 (100%) | 149 (40%) | 105 (15%) | 185 (52%) | 93 (28%) | |
| | | 147 (72%) | 95 (38%) | 171 (55%) | 81 (58%) | |
| | | 107 (100%) | 93 (17%) | 71 (86%) | 69 (100%) | |
| | | 81 (82%) | 83 (25%) | | | |
| | | | 81 (40%) | | | |
| | | | 36 (100%) | | | |

5S,6S-(7E,9E,11Z,14Z)-dihydroxyeicosatetraenoic acid (1), 15S-Hydroxy-(5Z,8Z,11Z,13E)-eicosatetraenoic acid (2) and 8R-Hydroxy-(5Z,9E,11Z,14Z)-eicosatetraenoic acid (3) (Table 6), α-tocopherol (4) γ-tocopherol (5), 13-(6-hydroxy-2,7,8-trimethylchroman-2-yl)-2,6,10-trimethyltridecanoic acid (6), 16-(4,5-dimethyl-3,6-dioxo cyclohexa-1,4-dienyl)-2,6,10,14-tetramethylhexadecanoic acid (7), 6-hydroxy-2,7-dimethyl-2-(4,8,12-trimethyltridecyl)chroman-8-carbaldehyde (8), 6-hydroxy-2,7-dimethyl-2-(4,8,12-trimethyltridecyl)chroman-8-carboxylic acid (9), calciferol (10), cholecalciferol (11), ergosterol (12), phylloquinone (13), retinol (l4) and 3β,7α-dihydroxy-5-cholestenoic acid (15).
*This terminology is specific to fatty acid fragmentation.

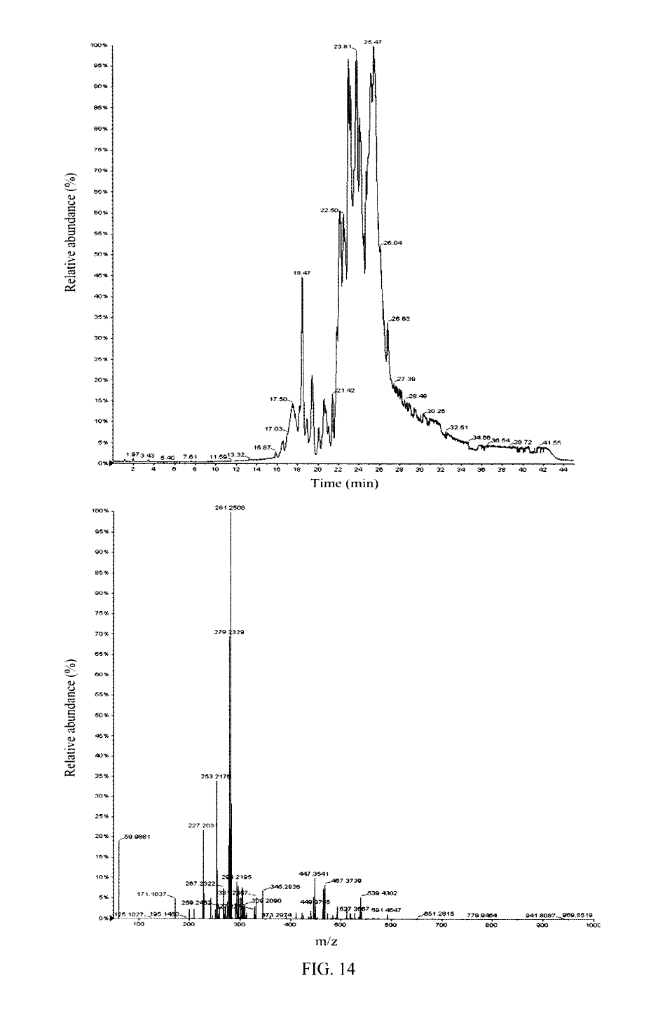

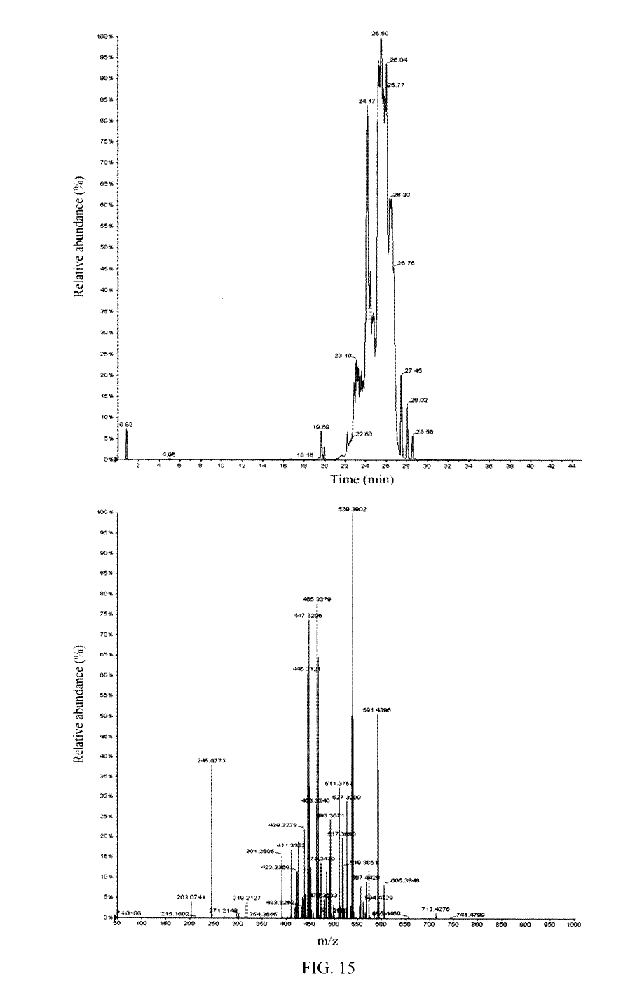

Next, an enrichment strategy using bulk serum extracts and a two-stage flash column chromatography approach followed by NMR analysis was carried out to provide further structural characterization of the hPULCFAs. First, reverse phase flash column chromatography (FCC) using a water-acetonitrile solvent gradient was performed and the resulting fractions analyzed by LC/MS. Fractions containing the hPULCFAs (fraction 9, FIG. 14) were pooled and subjected to normal phase FCC using chloroform-methanol mixtures to obtain an approximately 65% rich semi-purified fraction labeled sample A (See FIG. 15). LC and tandem mass spectrometric analyses (MS2 and MS3) data on sample A were used to track and confirm enrichment of the markers. Nuclear magnetic resonance (NMR, ¹H, ¹³C and ²D) analyses on sample A and its methyl esters revealed resonances and correlations (Table 6) consistent with very long chain polyunsaturated hydroxy fatty acids with observance of some suppression of resonances for hydrogen atoms attached to sp² carbons.

TABLE 6
¹H NMR data of CRC biomarker pool
(sample A) and their methyl esters

| | CRC biomarker | Methyl esters of CRC |
|---|---|---|
| Types of protons | pool | biomarker pool |
| CH₃ | 0.83-0.90 | 0.83-0.90 |
| CH₂ | 1.21-1.24, m | 1.21-1.24, m |
| —CH₂CH₂COOH | 1.57-1.65, m | 1.53-1.69, m |
| —CH₂CH═CH— | 1.98-2.08, m | 1.94-2.03, m |
| CH₂COO | 2.23-2.28, m | 2.23-2.31, m |
| —CH═CH—CH₂₋CH═ | 2.75-2.79, m | 2.74-2.82, m |
| OCH₃ | — | 3.64, s |
| —CH(OH)CH═ | 3.45-3.71, | 4.02-4.12, |
| | 4.03-4.26 | 4.16-4.26, 4.58-4.60 |
| —CH═ | 5.10-5.47, m | 5.08-5.40, m |
| —CH(OH)CH═ | 5.76-5.91, m | 5.75-5.90, m |

*NMR solvent is CDCl₃, signals assigned using 2D NMR experiments like HMQC and HMBC

### Independent Validation Using Multiple Reaction Monitoring (MRM) Methodology

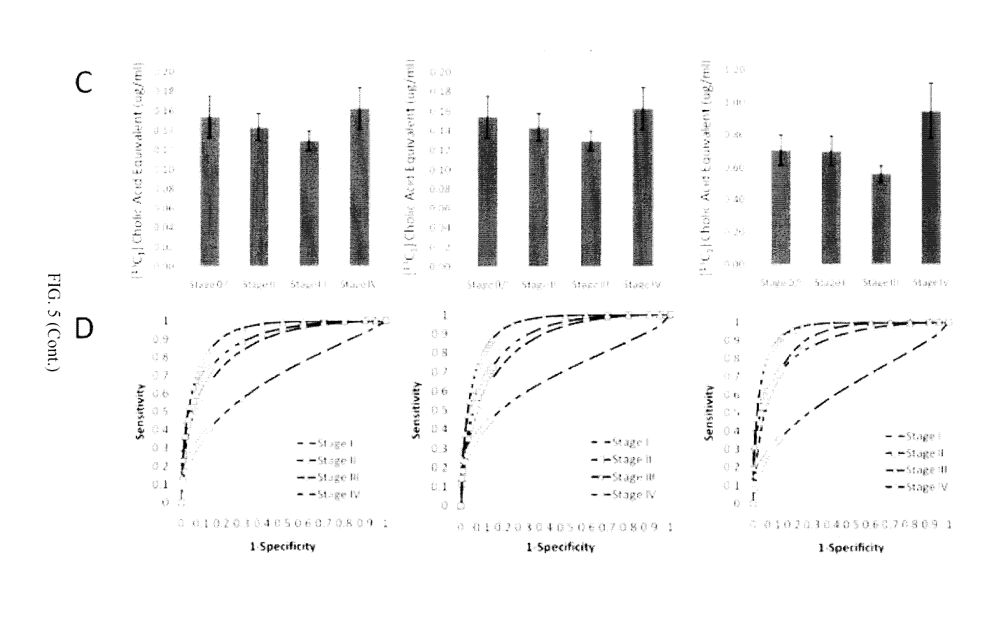

Reduced levels of hPULCFAs in the blood of CRC patients was further confirmed using a tandem mass spectrometry approach (see methods) in two more independent populations. The approach is based upon the measurement of parent-daughter fragment ion combinations (referred to as multiple-reaction monitoring; MRM) for quantifying analytes^{28,35}. We developed an assay to measure three of the 28 carbon hPULCFAs with four oxygens (parent masses 446, 448 and 450; C₂₈H₄₆O₄, C₂₈H₄₈O₄ and C₂₈H₅₀O₄, respectively) as described in the methods. Results are reported as equivalents to [¹³C₁]cholic acid (CAEs) spiked into each sample as an internal standard, since synthesis of labelled standards of the hPULFAs were still in progress at the time of the analysis. The first study comprised 70 treatment-naïve CRC subjects and 70 matched controls, all of which were Caucasians from the USA. The CAEs of the three 28-carbon hPULCFAs (named according to nominal mass 446, 448 and 450) for each subject are shown in FIG. 5A. Significantly lower levels (p<0.001, actual values shown in FIG. 5A) of each of the metabolites was observed in treatment-naive CRC-positive subjects compared to controls. ROC analysis resulted in AUCs of 0.87±0.005 for each of the 28-carbon containing hPULCFAs (FIG. 5B). Plotting patients by disease stage showed a slight further reduction between stage I and III, with stage IV subjects showing the least reduction (FIGS. 5C and 5D), albeit it only seven subjects. The corresponding average AUCs of the 28-carbon pool by stage were 0.87 for stage I, 0.88 for stage II, 0.94 for stage III, and 0.66 for stage IV.

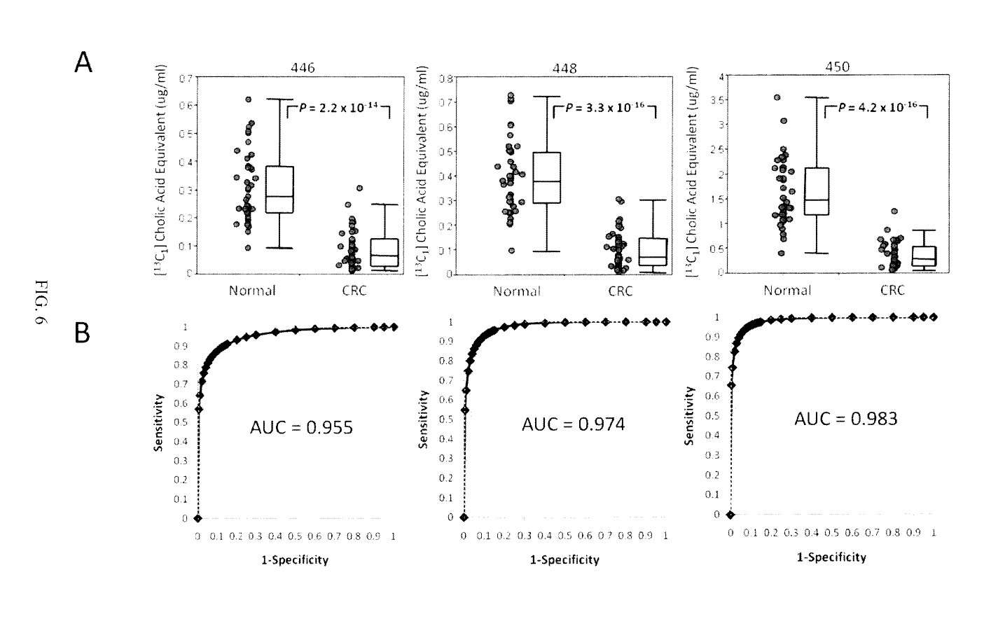

We next used the MRM method to characterize another independent population of CRC and control subjects from Chiba, Japan (Nomura et al). Serum from 40 pre-treatment CRC subjects and 40 controls were analyzed, and a significant reduction was again observed in the CRC-positive group (FIG. 6A). The corresponding average AUC for the three metabolites was 0.97±0.014 (FIG. 6B). In this study, a significant correlation with stage was observed (p<0.05) for all comparisons between stages I, II and III/IV (FIGS. 6C and 6D). The AUCs by stage were 0.93 for stage I, 0.97 for stage II, and 1.0 for stage III/IV (two stage IVs were grouped with stage III; FIG. 6D).

### Discussion

Described herein is the discovery and preliminary structural characterization of long-chain hydrocarbon-based metabolites harboring hydroxyl and carboxyl functional moieties, and containing between 28 and 36 carbons reduced in the serum of treatment-naive CRC patients compared to healthy asymptomatic controls. The utility of non-targeted metabolomics using high resolution FTICR-MS coupled with flow injection technology for biomarker discovery was tested by applying the technology to three independent test populations. In contrast to the “training/test-set” approach often used by splitting a single sample set in half to validate the performance of biomarkers³⁶⁻³⁸, which often relies on complex algorithms (see review³⁹) and can result in bias⁴⁰, we carried out fully independent discovery analyses on three separate sample sets matched cases and controls of different ethnic backgrounds collected from multiple sites around the world, to ensure a high degree of robustness and minimal chance of sampling bias. Of the top 50 metabolic discriminators discovered in the Osaka set, 44 and 47 of these were also significantly changed in the GCI and Seracare sets, respectively. This remarkable inter-study agreement indicates that not only is non-targeted FTICR-MS technology a reproducible biomarker discovery engine, but that disease-related metabolomic changes can be highly conserved across geographic locations and races. The translation of the non-targeted FTICR-MS discoveries into a simple assay was also tested by developing a targeted TQ-MRM method for two biomarker candidates, using this simplified method on two further independent test populations, and then comparing the ROC AUCs generated from the 3 FTICR-MS studies with the ROC AUCs generated from the TQ-MRM method. Similar results were obtained using the simplified method. In total, five independent study populations collectively comprising 222 treatment-naive CRC patient samples and 220 disease-free asymptomatic controls were evaluated using two different analytical methods. Indeed, the likelihood of the reported association between the reduction of hPULCFAs and CRC being a false positive result across the five independent sets of samples is astronomically low. Meta-Analysis was performed on the false positive rates using Fisher's Inverse Chi-square Method (Reject H₀ if P=−2 Σ^{k}ᵢ₌₁ log pᵢ>C; p=P-values of five independent samples, k=five different samples, C=upper tail of the chi-square distribution with 2 k degrees of freedom (X²_{0.05, 10}=18.31))^{41,42}. Based upon the meta-analysis, the resulting p-values for markers 446 and 448 were more significant than the individual p-values, at 2.96×10⁻⁴⁷ and 8.11×10⁻⁴⁹, respectively. We can therefore say with a high degree of confidence that a reduction in these metabolites correlates with the presence of CRC.

The FTICR-MS provided resolution sufficient for confident molecular formula predictions based upon accurate mass in conjunction with extraction, ionization, and statistical correlative information. Although multiple elemental compositions were theoretically assignable to given biomarker masses, only formulas having 28 to 32 carbons, and four to six oxygen were consistently assignable to common masses detected in two or three of the discovery sets. Given a high degree of statistical interaction between the sample-to-sample expression profiles of the hPULCFAs (i.e., a high degree of correlation between the relative intensities of the markers across subjects) we suspected they were all part of the same metabolic system and should therefore show related compositions. Detection in negative ionization mode also reduced the likelihood that nitrogen was present in any of the compositions. This information in conjunction with tandem mass spectrometry showing prominent losses of water and carbon dioxide led us to confidently propose the molecular formulas shown in Table 3 and Table 2.1. A number of candidate classes of molecules which theoretically fit the molecular formula class were also excluded using tandem MS. For example, we observed no fragments indicative of condensed ring systems such as those in steroids or vitamin D, and no fragments indicative of chroman ring systems such as those observed in the vitamin E tocopherols. Several other classes of molecules including vitamin K and retinol, and bile acids such as cholic acid and 3β,7α-dihydroxy-5-cholestenoic acid also did not show comparable fragmentation patterns. However, the similarity in fragmentation pattern, particularly in the relative abundances of daughter ions resulting from losses of CO₂ and H₂O, and chain cut ions from the hPULCFAs to known hydroxy fatty acid standards as well as other fatty acids reported in the literature such as the resolvins and protectins (discussed below), suggested hydroxylated long-chain fatty acid-type species. Examination of the tandem-MS data for the C28 series (masses 446, 448, 450, 464, 466 and 448) revealed a consistent 113 Da daughter ion, which we reasonably predict to represent the carboxy-terminus chain fragment —CH₂—CH═CH—CH₂—CH₂—COOH. In addition, a consistent loss of 54 (—CH═CH—CH₂—CH₂—) from the [M-(CO₂+H₂O)] daughter ion was observed for the 446, 448, 464, and 466, but not the 450 and 468 molecules, suggesting that 1), the 450 and 468 may have a saturated carboxy terminal region, and 2), that there are likely no hydroxyl moieties within this region of the molecule. MS/MS data of all the C28 and other markers also did not show the diagnostic fragment obtained with a 1,2-diol motif as observed for 1 (base peak is chain cut ion at m/z 115) and NMR on fractions enriched via flash-column chromatography showed lower than expected integration values obtained for the ¹H NMR signals at δ 2.78 (methylene interruptions between double bond carbons) and at δ 5.12-5.90 (hydrogen atoms on double bond carbons). Cumulatively these results suggested that the hydroxy groups in the molecules are likely bonded to the carbon atoms between the sp² carbons at least seven carbons from the carboxy end.

Based on the structural analysis, structures for the six C28 biomarkers have been proposed as shown below in Table 6.1:

TABLE 6.1
CRC Biomarkers and Proposed Structures

| Biomarker | Mass and Formulae | Structure |
|---|---|---|
| 446 | Chemical Formula: C₂₈H₄₆O₄ Exact Mass: 446.3396 | 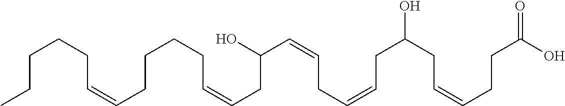 |
| 448 | Chemical Formula: C₂₈H₄₈O₄ Exact Mass: 448.3553 | 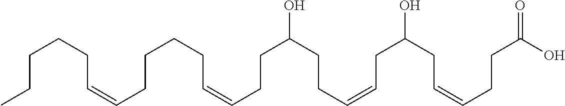 |
| 450 | Chemical Formula: C₂₈H₅₀O₄ Exact Mass: 450.3709 | 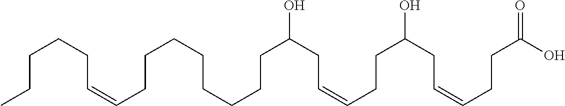 |
| 464 | Chemical Formula: C₂₈H₄₈O₅ Exact Mass: 464.3502 | 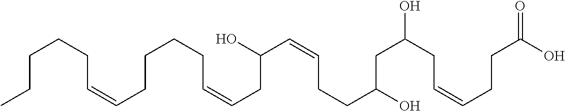 |
| 466 | Chemical Formula: C₂₈H₅₀O₅ Exact Mass: 466.3658 | 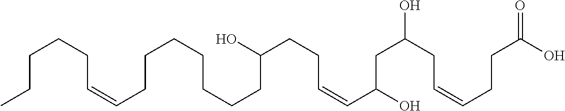 |
| 468 | Chemical Formula: C₂₈H₅₂O₅ Exact Mass: 468.3815 | 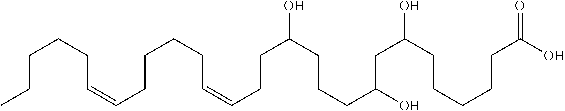 |

Interestingly, the metabolite markers reported herein represent a human-specific metabolic system. We analyzed serum samples from multiple species, including rat, mouse, and bovine, as well as multiple different sample sources including numerous cell lines, conditioned media, tumor and normal colonic tissue from patients in the GCI discovery set, and brain, liver, adipose, and other tissues from various species, all of which failed to show any detectable levels of these hPULCFAs (results not shown). We also could not detect these molecules in various plant tissues or grains, including policosanol extracts which are rich in saturated C28 and longer-chain fatty acids^{43, 44}. This suggests that the molecules originate from human-specific metabolic processes, such as specific p450-mediated and/or microbiotic processes. The lack of detection in tumor or normal colonic tissue suggests that the metabolites are not “tumor markers”, and combined with the high rate of association in stage I cancer, it is not likely that the reduction is the result of tumor burden. However, the further reduction of levels observed in some late stage Japanese cases (FIG. 6) could be explained if lower levels of the hPULCFAs were indeed indicative of progression rate in this group. It is also important to note that in all control groups reported herein, subjects were not colonoscopy-confirmed to be free of tumors or advanced neoplasia. Based upon colonoscopy results by Collins et al in average-risk subjects, up to 10% of an asymptomatic population is positive for advanced neoplasia⁴⁵. Therefore, the ability of these metabolites to discriminate between subjects at risk and not at risk for CRC is likely under-estimated in our results.

Although fatty-acid molecules of this length containing hydroxyl groups have not previously been reported, they appear to resemble a class of hydroxylated very long-chain fatty acids known as the resolvins and protectins that originate from the n3 essential fatty acids EPA and DHA, respectively, which are critical in promoting the resolution of acute inflammation. The inability to sufficiently “resolve” acute inflammation is the leading theory behind the establishment of chronic inflammatory states which underlie multiple conditions including cancer⁴⁶ and Alzheimer's Disease⁴⁷. Of particular relevance is the effect of pro-resolution long-chain hydroxyl fatty acid mediators on intestinal inflammatory conditions such as IDB, Crohn's Disease, Colitis, and colon cancer. Both Resolvin E1 (RvE1) and Lipoxin A4 (LXA4) have been implicated with protective effects against colonic inflammation. RvE1 was shown to protect against the development of 2,4,6-trinitrobenze sulfonic acid-induced colitis in mice, accompanied by a block in leukocyte infiltration, decreased proinflammatory gene expression, induced nitric oxide synthase, with improvements in survival rates and sustained body weight⁴⁸. Similarly, LXA4 analogues have been shown to attenuate chemokine secretion in human colon ex vivo⁴⁹, and attenuated 50% of genes, particularly those regulated by NFκB, induced in response to pathogenically induced gastroenteritis⁵⁰. In vivo, LXA4 analogues reduced intestinal inflammation in DSS-induced inflammatory colitis, resulting in significantly reduced weight loss, hematochezia and mortality⁵⁰. Structurally, resolvins and protectins (as well the n6 lipoxins) comprise mono-, di- and tri-hydroxylated products of the parent VLCFAs, catalyzed by various lipoxygenases, cyclooxygenases and p450 enzymes⁵¹⁻⁵⁵.

The utility of the diagnostic methods described herein is supported by the consistently observed reduction of the hPULCFAs in CRC patients. In addition, the average AUC across all the case-control data reported here was 0.91±0.04, which translates into approximately 75% sensitivity at 90% specificity with little to no disease-stage bias. Because the metabolites are measured in serum, compliance should be high, and the test can be cost-effectively run on standard triple-quadrupole mass spectrometers or the like in a similar manner as the inborn errors of metabolism tests²⁸.

### 2. Analysis of a Biological Role for hPULCFAs

### Materials and Methods

Cell lines: SW620, MCF-7 and RAW264.7 were purchased from ATCC and cultured in high glucose DMEM, 10% FBS at 37° C., 5% CO₂.

Quantitative Real time PCR: RAW264.7 cells were seeded at 1×10⁶/well in E-well plate the day before the treatment. The following day, the cells were treated with different concentration of hPUCLFA(D046) or 1% FA/DMSO (DMSO) as vehicle control for 4 hours; each treatment was in duplicate; then stimulated with LPS at 1 μg/ml (cat. No. L4391, Sigma) for 20 hours. Total RNA was isolated from cell pellets using Trizol (Cat. No. 15596-018, Invitrogen) as per manufacturer's instruction. The RNA pellets were resuspended in 50 μL of DEPC treated water and stored at −80° C. RNA concentration and purity was determined by spectrophotometry at 260 and 280 nm. Reverse transcription was performed using qScript cDNA super mix (Cat No. 95048-100, Quanta Biosciences); PCR was conducted by using Fast SYBR Green Master Mix (Cat No. 4385612, AB Applied Biosystems) on an Applied Biosystems Step one Plus Real-time PCR system. Real-time PCR used primers are listed below. The relative number of each transcript copy was normalized by house-keeping gene Beta Actin.

| Gene | Amplicon | Sequences (5′ to 3′) |
|---|---|---|
| iNOS forward | 226 bp | CACCTTGGAGTTCACCCAGT |
| (SEQ ID NO: 1) | | |
| iNOS reverse | | ACCACTCGTACTTGGGATGC |
| (SEQ ID NO: 2) | | |
| COX2 forward | 191 bp | CCCCCACAGTCAAAGACACT |
| (SEQ ID NO: 3) | | |
| COX2 reverse | | CTCATCACCCCACTCAGGAT |
| (SEQ ID NO: 4) | | |
| TNFalpha forward | 188 bp | AGAAGTTCCCAAATGGCCTC |
| (SEQ ID NO: 5) | | |
| TNFalpha reverse | | GTCTTTGAGATCCATGCCGT |
| (SEQ ID NO: 6) | | |
| IL1 beta forward | 175 bp | TGTGAAATGCCACCTTTTGA |
| (SEQ ID NO: 7) | | |
| IL1 beta reverse | | TGAGTGATACTGCCTGCCTG |
| (SEQ ID NO: 8) | | |

Nitrite: Nitrite concentration was measured by Griess Reagent (Cat. No. G2930, Promega). The RAW cells or SW620 cells were treated as described in real time PCR. Conditioned medium was collected for Nitrite measurement. The measurement was conducted as per manufacture's instruction in 96 well plate.

ELISA for mouse TNF alpha: Raw cells were treated as described in real time PCR. Conditioned medium was collected. Cells was briefly washed with ice cold PBS and lysed with lysis buffer (Jerry's recipe); Protein in the cell lysate was quantified using the Bio-Rad Protein Assay (Bio-Rad, Hercules, Calif.). 50 ul of conditioned medium or 100 ug of cell lysate per well was used to determine the amount of TNF alpha as per manufactory's instruction (Cat. No. KMC3011, Invitrogen).

ELISA for mouse IL-1 beta: Raw cells were treated as described in real time PCR. 50 ul of conditioned medium or 100 ug of cell lysate was used to determine the amount of IL-1 beta as per manufactory's instruction (Cat. No. MLB00B, Quantikine)

Western Analysis: Cells were removed from 100 mm tissue culture plates with a rubber policeman in chilled PBS, collected by centrifugation at 4° C., and resuspended in 100 ul in ice cold lysis buffer (50 mM Tris pH 7.5, 150 mM NaCl, 0.1% NP-40, 0.5 mM EDTA, 0.1 mM EGTA plus 1X-Sigma mammalian cell anti-protease cocktail). The cells were lysed using multiple freeze-thaw cycles followed by pulse sonication and high-speed centrifugation at 4° C. to remove cell debris. For Western analysis equivalent amounts of protein (assessed by Bradford protein assay using Biorad Protein Reagent) were resolved by SDS-PAGE. Following electrophoresis the proteins were trans-blotted onto nitrocellulose membranes (Pall-VWR). The membranes were blocked over night on a gyratory plate at 4° C. with 5% molecular grade fat free skim milk powder (Biorad Laboratories, Mississauga ON Canada) in phosphate-buffered saline (PBS) containing 0.1% Tween-20. Primary antibodies from Santa Cruz Biotechnology were incubated at a 1:1000 dilution overnight at 4° C. and secondary HRP antibodies were applied at a 1:10000 dilution for 30 min. at RT. Subsequent washes were carried out in the same buffer. An enhanced chemiluminescence (ECL) detection system (Dupont-NEN) was used to detect the antigen/antibody complexes. Blots were exposed to BioMax chemiluminescent X-ray film (Kodak) and target signals were scanned and quantified using a HP scanner (Scanjet G4010) with ImageJ densitometric software.

Generation of hPULCFA-Enriched Extract

Ethyl acetate extracts of normal human serum containing hPULCFAs (180 mL serum, 500 mg extract) were subjected to reverse phase flash column chromatography with a step gradient elution; acetonitrile-water 25:75 to 100% acetonitrile. It is noted here that other similar extraction methods could also be used, and purification and/or enrichment could also be performed using other chromotagraphic approaches, for instance but not limited to high performance liquid chromatography (HPLC). The flash column fractions were collected and analyzed by LC/MS and MS/MS. The fractions containing the CRC biomarkers were pooled (12.5 mg). Fractions were monitored for hPULCFAs by subjecting the sample to HPLC (HP 1100 with Hypersil™ ODS 5 μm, 125×4 mm column, Agilent Technologies)-coupled time-of-flight mass spectrometry using an ABI QSTAR® XL mass spectrometer fitted with an APCI source in negative mode. Any equivalent mass spectrometer could also be used. The scan type in full scan mode was time-of-flight (TOF) with an accumulation time of 1.0000 seconds, mass range between 50 and 1500 Da, and duration time of 55 min. Source parameters were as follows: Ion source gas 1 (GS1) 80; Ion source gas 2 (GS2) 10; Curtain gas (CUR) 30; Nebulizer Current (NC) −3.0; Temperature 400° C.; Declustering Potential (DP) −60; Focusing Potential (FP) −265; Declustering Potential 2 (DP2) −15. In MS/MS mode, scan type was product ion, accumulation time was 1.0000 seconds, scan range between 50 and 650 Da and duration time 55 min. All source parameters are the same as above, with collision energy (CE) of −35 V and collision gas (CID, nitrogen) of 5 psi.

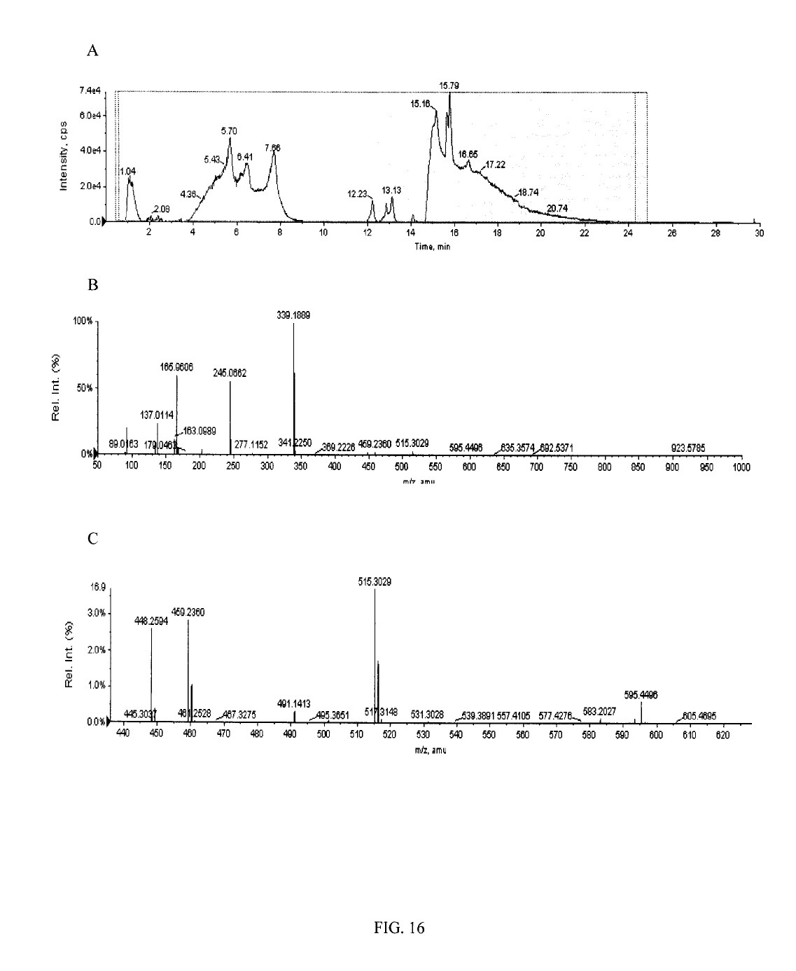

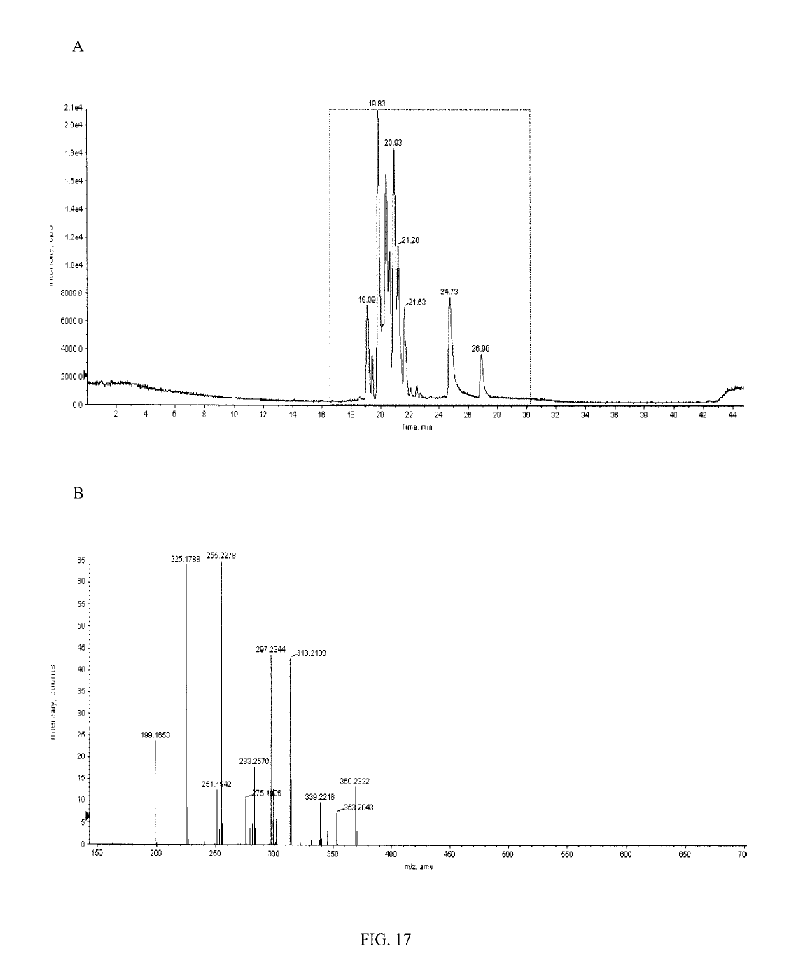

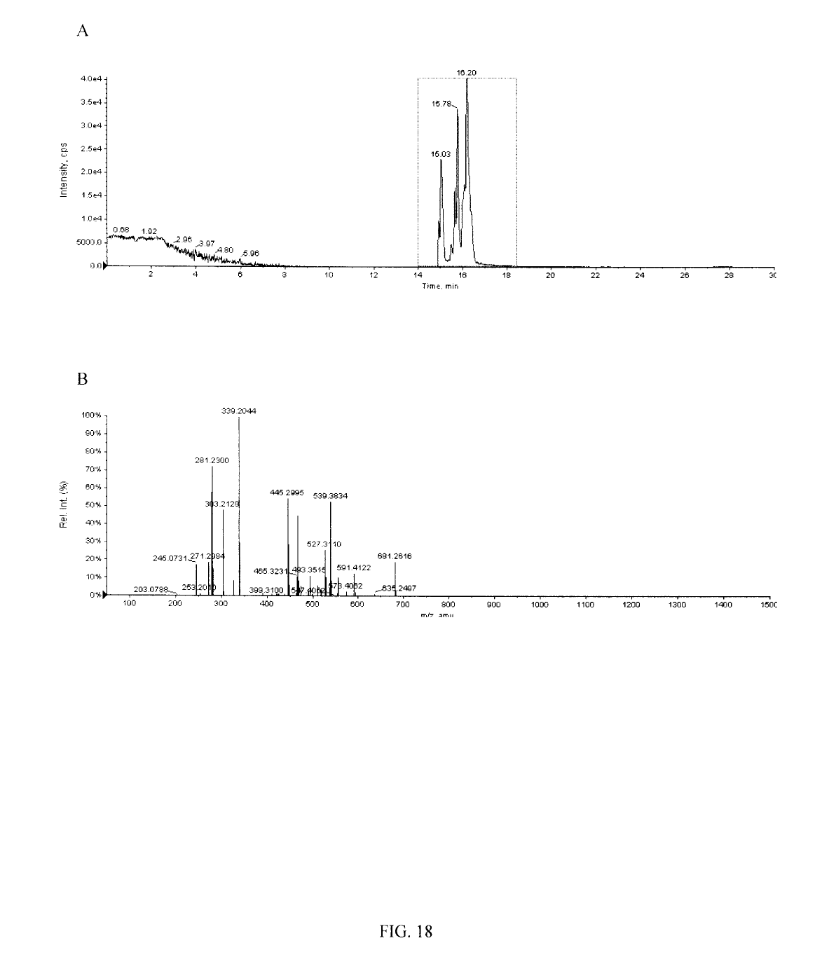

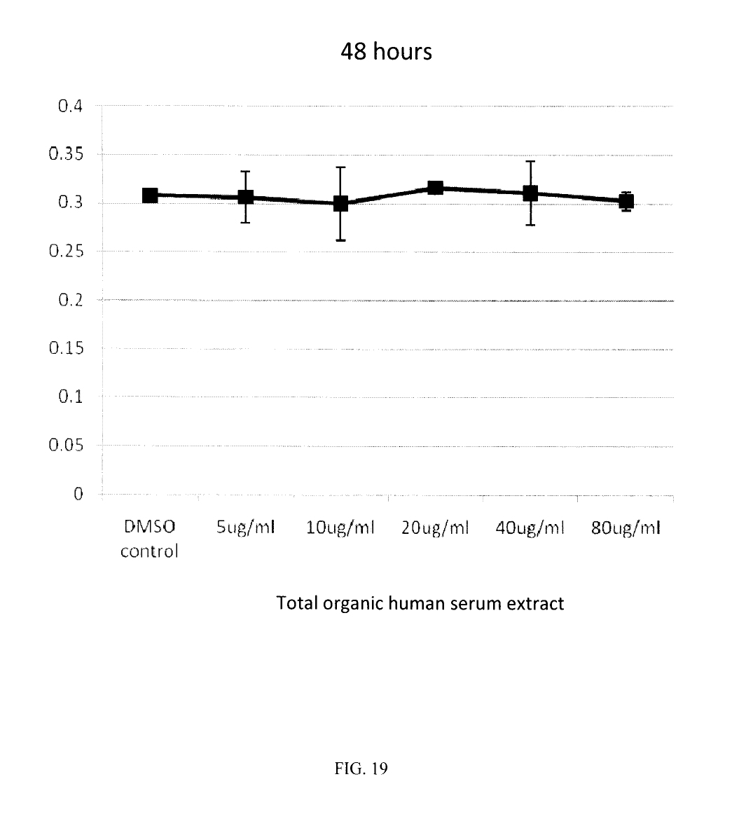

The results of the flash column enrichment of hPULCFAs is seen in FIGS. 16, 17 and 18. Serum components, such as dietary and shorter-chain fatty acids can be removed, resulting in a semi-purified extract containing concentrated levels of hPULCFAs.

Biological Activity of hPULCFAs

Biological activity of hPULCFAs was determined by A). Assessing the activity of hPULCFA-enriched extracts relative to extracts depleted of hPULCFAs using various cell-based systems, and B). Synthesizing and determining the activity of a specific hPULCFA.

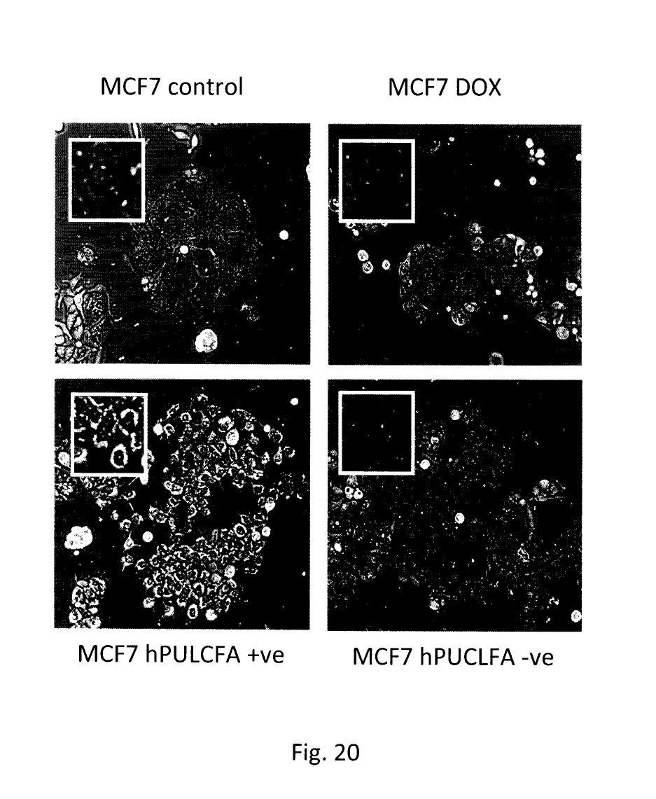

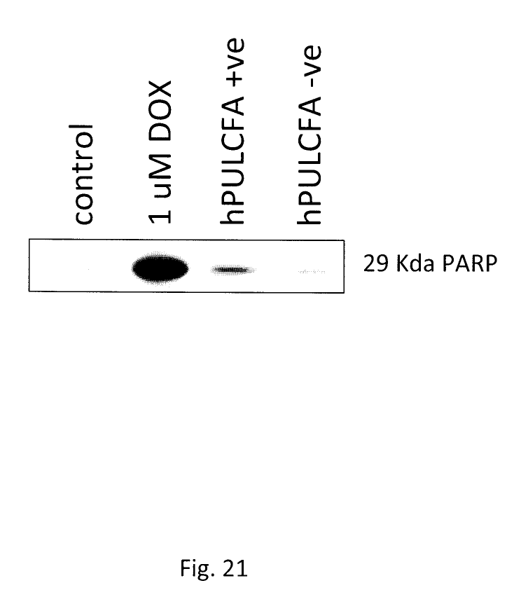

MFC human breast carcinoma cells treated with 80 ug/ml hPULCFA-positive extracts resulted in morphological transformations typical of apoptotic cells including increased granularity, apoptosomes and irregular nuclei (FIG. 20) which were not observed in cells treated with hPULCFA-negative extract or vehicle (controls). The number of viable cells was also visually lower in the hPULCFA-treated cells (FIG. 20). Western blot analysis confirmed the presence of caspase activity in hPULCFA-treated MCF cells as assessed through the appearance of the 29 kDa poly-ADP ribose polymerase (PARP) cleavage product (FIG. 21).

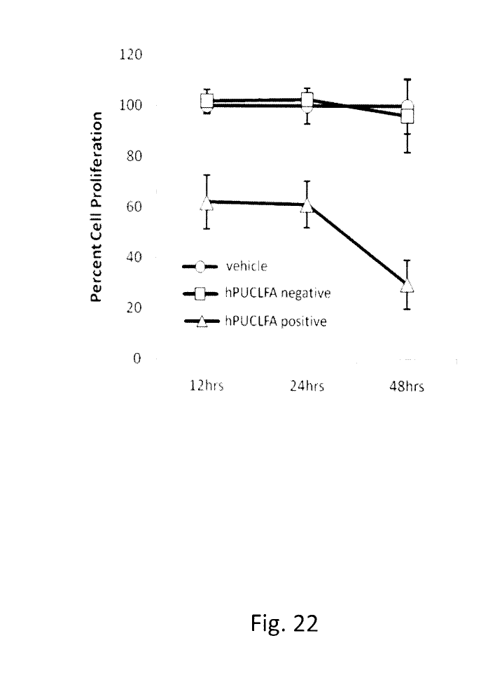

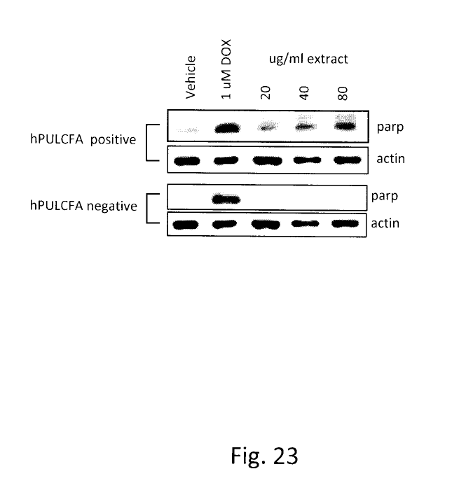

In a similar fashion, SW620 colon cancer cells treated with 80 ug/ml serum extract enriched with hPULCFAs showed a 40% reduction in cell proliferation at 12 hours, and 70% reduction by 48 hours which was not observed for control or vehicle extracts (FIG. 22). Similar to the MCF 7 cells, light microscopy of the cells suggested a possible pro-apoptotic effect associated with reduced proliferation (not shown). As shown in FIG. 23, PARP activity was detectable with hPULCFA-enriched extracts but not control extract or vehicle. Collectively the results suggest a functional role of hPULCFAs in inducing apoptosis.

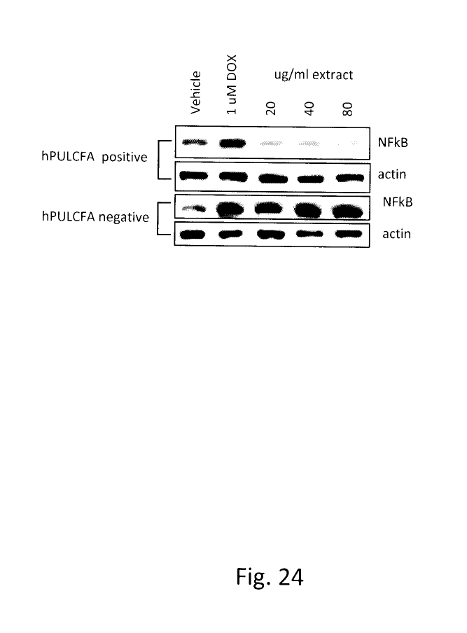

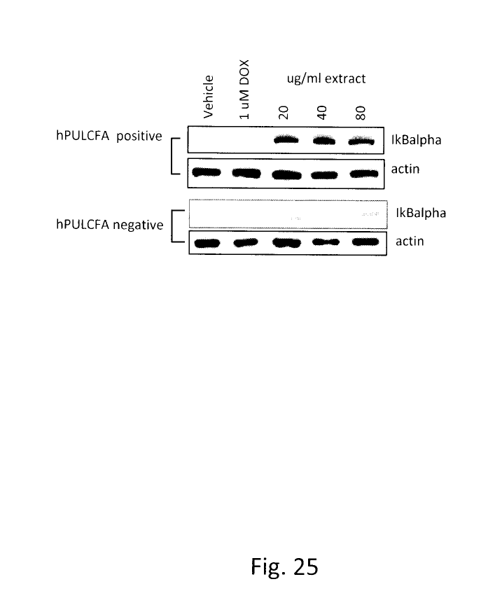

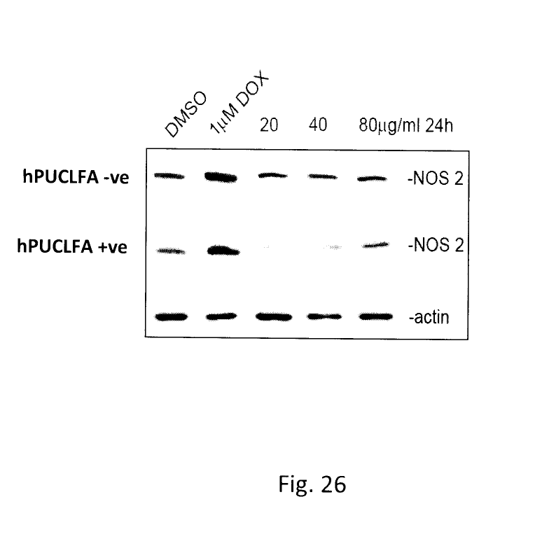

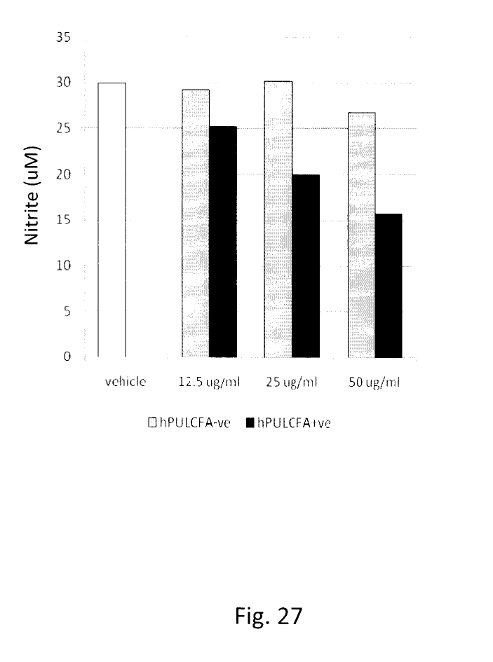

The effect of hPULCFA extract on a series of inflammatory proteins was next investigated by immunoblots. Treatment of SW620 cells with hPULCFA enriched extracts resulted in a reduction of the pro-inflammatory transcription factor NFκB (FIG. 24), with a simultaneous induction of IκBα (FIG. 25), the negative regulator of NFκB. In addition, hPULCFA-enriched extracts showed an inhibitory effect on inducible nitric oxide synthase (iNOS, or NOS2, FIG. 26), which is normally induced in inflamed tissues generating large amounts of nitric oxide that can promote mutagenic changes through DNA oxidation and protein nitrosylation. The generation of nitric oxide in hPULCFA-treated cells was subsequently determined through the measurement of reduce nitrite levels in conditioned media (FIG. 27). Nitrite is a stable metabolite of nitric oxide, which can react with various organic compounds forming nitrosamines and other nitrate radicals that can be mutagenic. Notably, NO is induced during various inflammatory responses such as bacterial infections, and has been directly implicated as a cause of colon cancer (Erdman et al, PNAS, Jan. 27, 2009, vol 106 No. 4). The reduction of iNOS(NOS2) as well as nitrite in hPULCFA-treated cells suggests that hPULCFAs can inhibit this pro-inflammatory process.

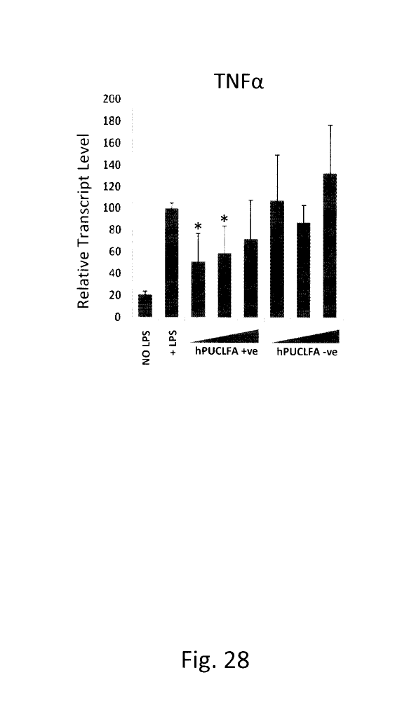

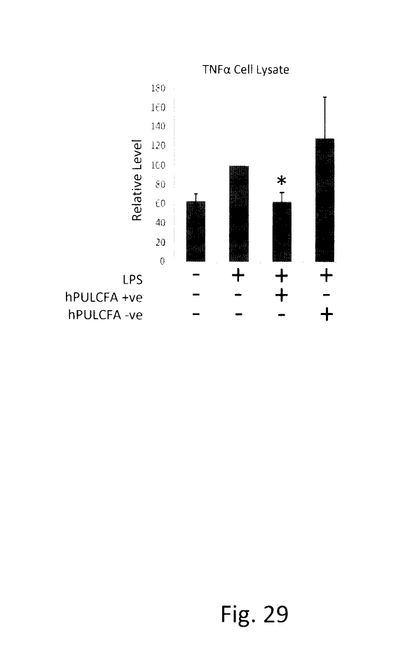

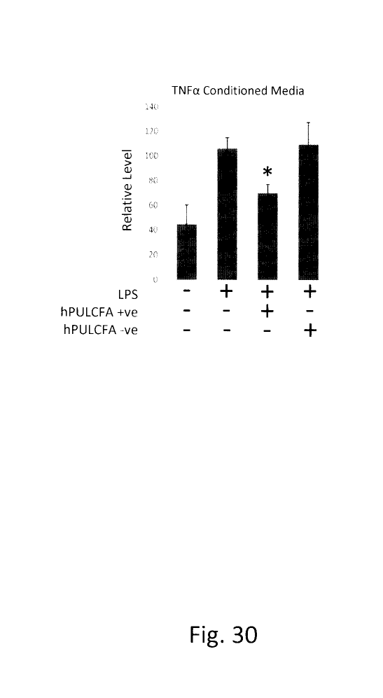

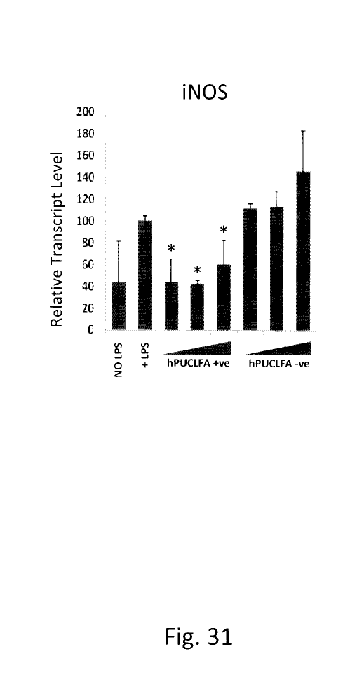

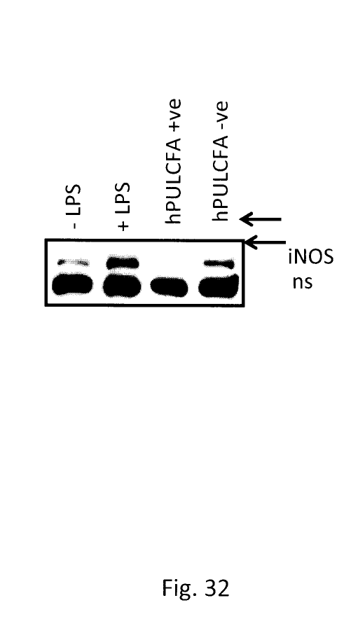

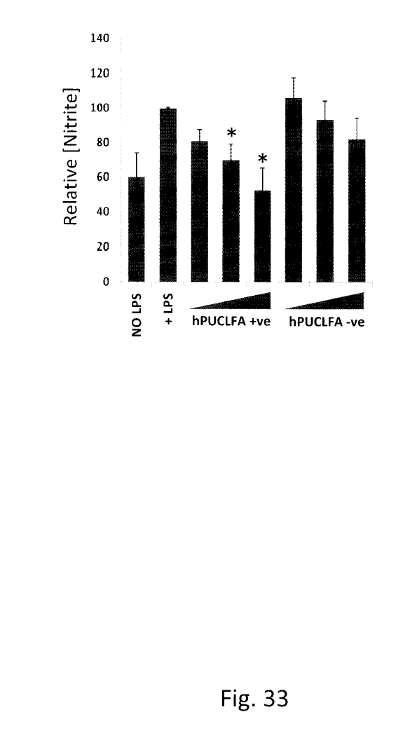

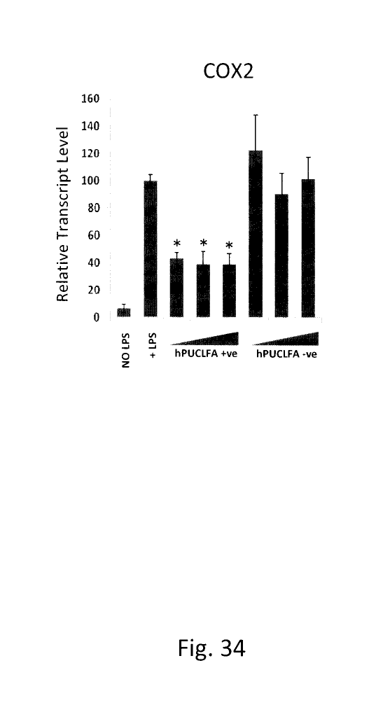

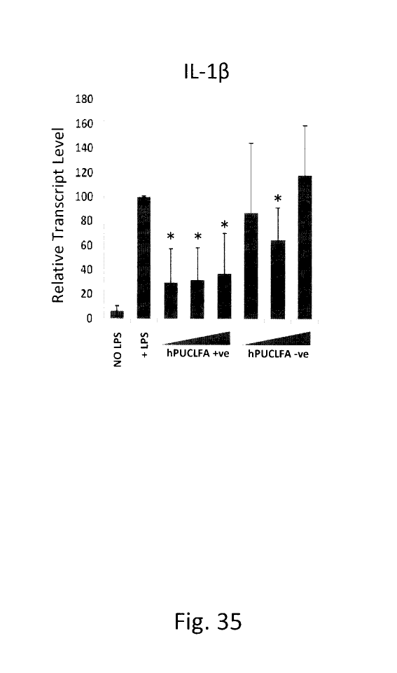

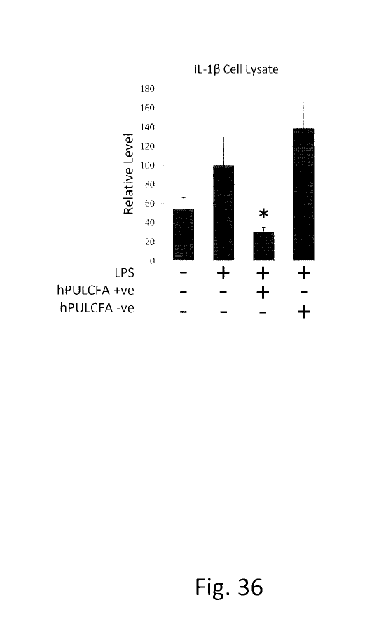

The RAW293 mouse macrophage cell model system is commonly used to assess anti-inflammatory activity of compounds. The cells are treated with lipopolysaccharide which induces a massive inflammatory response, of which compounds can be tested for their ability to protect against. RAW293 cells were pretreated with hPULCFA-enriched extract followed by treatment with LPS for 24 hours after which mRNA transcript and protein levels of pro-inflammatory markers including the cytokines tumor necrosis factor alpha (TNFα) and interleukin-1 beta (IL-1β), iNOS as described above, cyclooxygenase 2 (COX2, the enzyme responsible for the production of pro-inflammatory eicosanoids from arachidonic acid) were assessed. Following treatment with LPS, levels of TNFα mRNA transcript levels showed a statistically significant reduction (p>0.05) in cells exposed to hPULCFA-enriched extracts compared to control extracts (FIG. 28). Levels of TNFα protein, as assessed by enzyme-linked immunosorbant assay (ELISA) in cell lysates (FIG. 29) as well as conditioned media (FIG. 30) were also significantly reduced (p<0.05) in hPULCFA-treated cells compared to controls. hPULCFA treatment also blocked the LPS-mediated induction of iNOS mRNA compared to control treatments (p<0.05; FIG. 31), which corresponded with a significant reduction in iNOS protein as assessed by immunoblot (FIG. 32), and a dose-dependent inhibition of nitric oxide production as determined by nitrite levels (FIG. 33). mRNA transcript levels of COX2, as shown in FIG. 34, were also significantly reduced in hPULCFA-treated cells versus controls (p<0.05), as were mRNA transcript levels (FIG. 35; p<0.05) and cell lysate protein levels (FIG. 36; p<0.05) of IL-1β. Collectively these results illustrate the utility of hPULCFAs in protecting against a pro-inflammatory state.

A hPULCFA (named D046-124) with molecular formula C28H46O4 of the following structure:

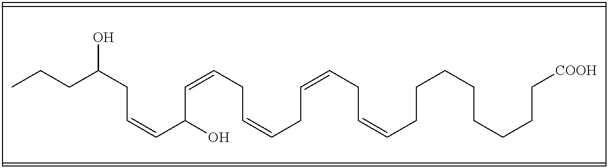

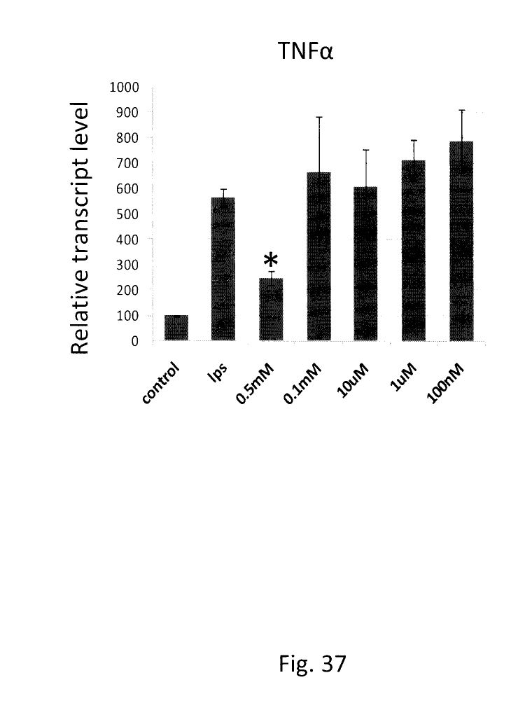

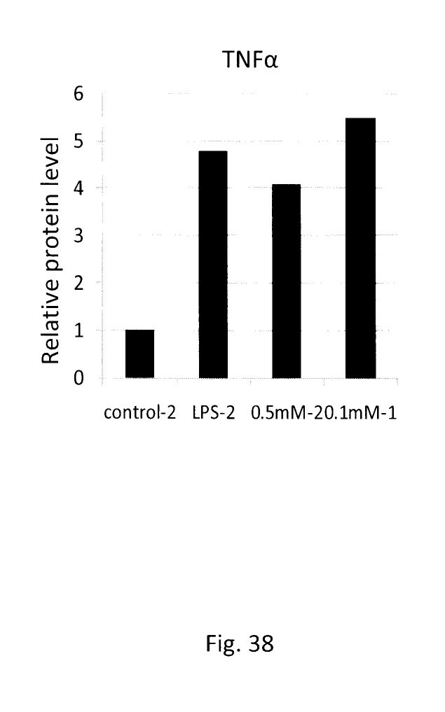

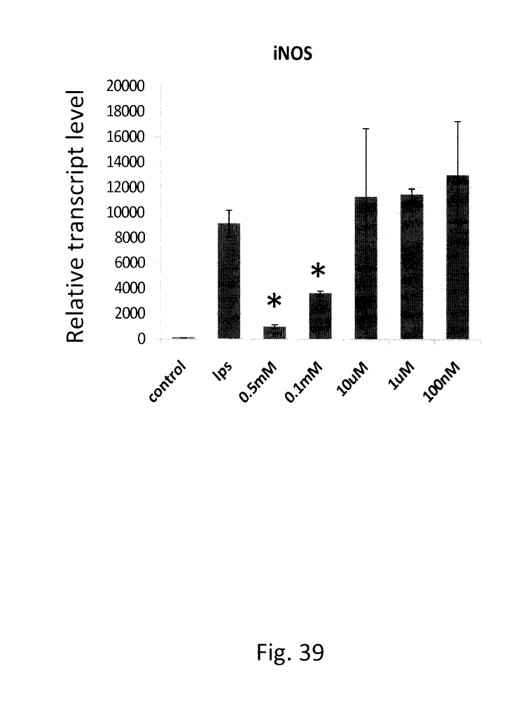

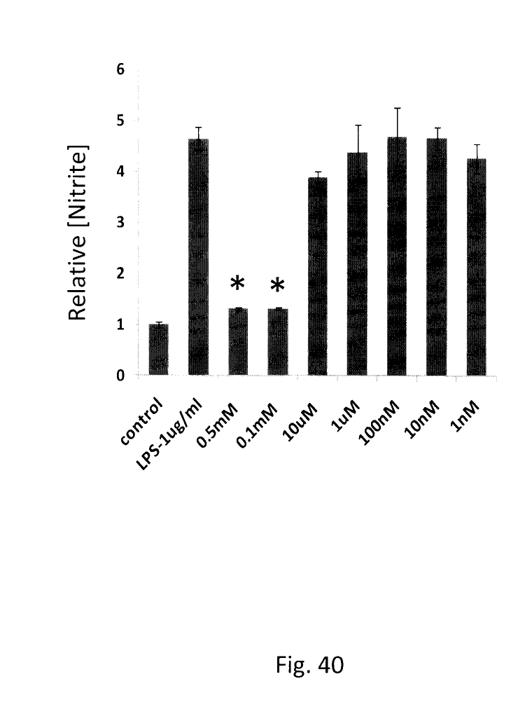

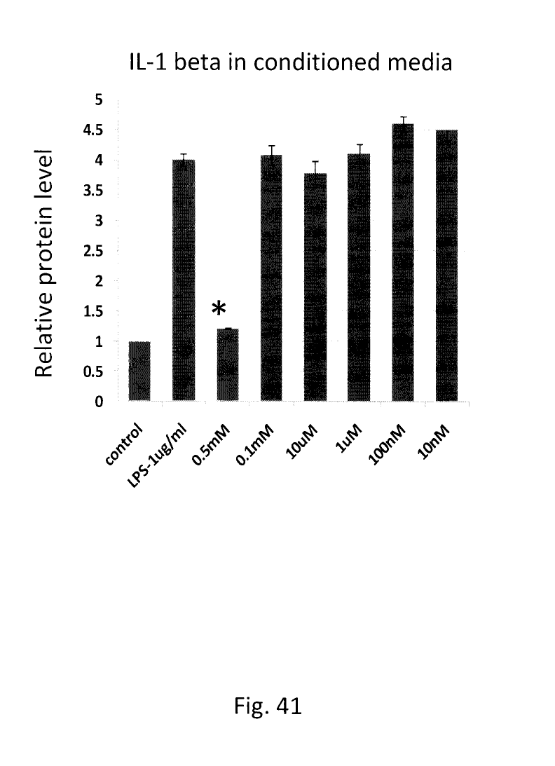

was synthesized to 98.7% purity (as assessed by LCMS) according the synthetic scheme described in Example 3 (below). Treatment of RAW293 cells with the pure hPULCFA prior to LPS stimulation prevented the induction of TNFα transcripts at 500 uM (0.5 mM) as shown in FIG. 37 (p<0.05) and protein level in conditioned media at 0.5 mM as shown in FIG. 38. Similar inhibitory effects were observed for mRNA transcript levels of iNOS (p<0.05) at doses of 0.5 and 0.1 mM (FIG. 39) as well as for nitric oxide as determined through nitrite levels at the same concentrations (p<0.05, FIG. 40). Similar effects were also observed for levels of IL-1β in conditioned media, for which LPS-mediated stimulation was completely blocked by 0.5 mM of the pure hPULCFA (FIG. 41, p<0.05).

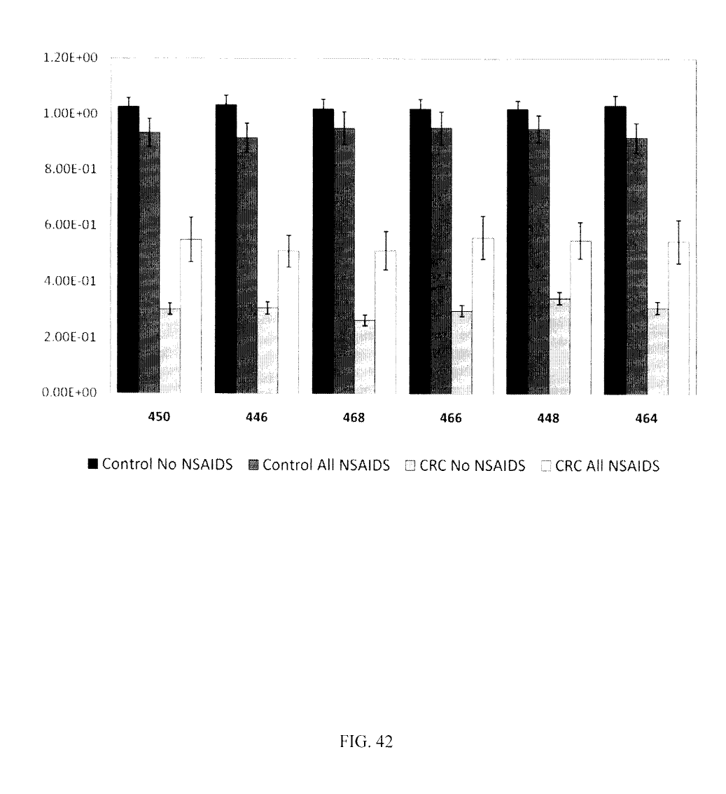

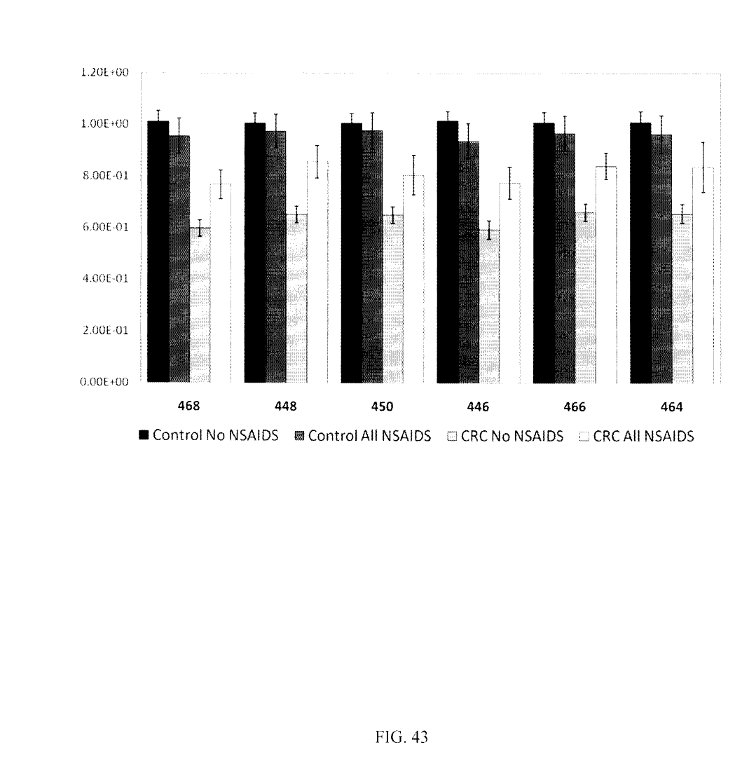

To further explore the anti-inflammatory role of the hPULCFAs, levels of six hPULCFAs (450 Da, 446 Da, 468 Da, 466 Da, 448 Da and 464 Da) were measured in two large populations of CRC and healthy subjects taking NSAIDs. As can be seen in FIGS. 42 and 43 (for which the respective population details can be seen in Tables 7 and 8), a statistically significant and reproducible increase in hPULCFA levels arises in CRC subjects taking non-steroidal anti-inflammatory drugs. This effect is observed in both treatment-naïve CRC patients (Table 7, FIG. 42) and CRC patients following treatment (Table 8, FIG. 43), and shows that use of NSAIDs result in the increase of hPULCFA levels in deficient subjects. This effect is not, however, observed in subjects who already have normal hPULCFA levels. Accordingly, measuring hPULCFA levels can be used to monitor the effects of NSAIDs in a treatment regime.

TABLE 7
Population distribution of subjects in treatment-naive
population tested for NSAID Effects on six hPULCFAs.

| | |
|---|---|
| Control No NSAIDS | 207 |
| Control All NSAIDS | 82 |
| ASA | 46 |
| Ibuprophen | 19 |
| ASA/Ibuprophen | 5 |
| Celecoxib | 2 |
| Excedrin/Ibuprophen | 2 |
| Ibuprophen/Naproxen Sodium | 1 |
| Naproxen Sodium | 5 |
| Refecoxib | 2 |
| CRC No NSAIDS | 151 |
| CRC All NSAIDS | 37 |
| ASA | 24 |
| Ibuprophen | 7 |
| ASA/Ibuprophen | 2 |
| Refecoxib | 1 |
| ASA/Refecoxib | 1 |
| Celecoxib | 1 |
| Excedrin | 1 |

TABLE 8
Population distribution of subjects in CRC patients following
treatment, and tested for NSAID Effects on six hPULCFAs.

| | |
|---|---|
| Control No NSAIDS | 202 |
| Control All NSAIDS | 48 |
| ASA/Ibuprophen | 2 |
| ASA | 27 |
| Celecoxib | 1 |
| Excedrin/Ibuprophen | 1 |
| Excedrin | 2 |
| Ibuprophen/Naproxen Sodium | 1 |
| Ibuprophen | 13 |
| Refecoxib | 1 |
| CRC No NSAIDS | 187 |
| CRC All NSAIDS | 80 |
| ASA | 36 |
| ASA/Celecoxib | 2 |
| ASA/Ibuprophen | 3 |
| ASA/Refecoxib | 1 |
| Celecoxib | 3 |
| Celecoxib/Ibuprophen | 2 |
| Excedrin | 3 |
| Ibuprophen | 22 |
| Ibuprophen/Refecoxib | 1 |
| Naproxen Sodium | 3 |
| Refecoxib | 4 |

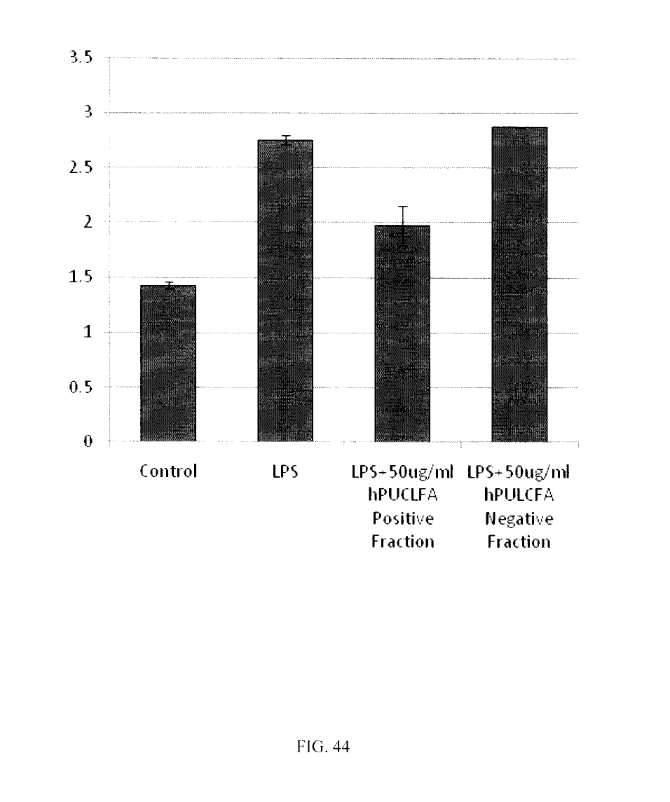

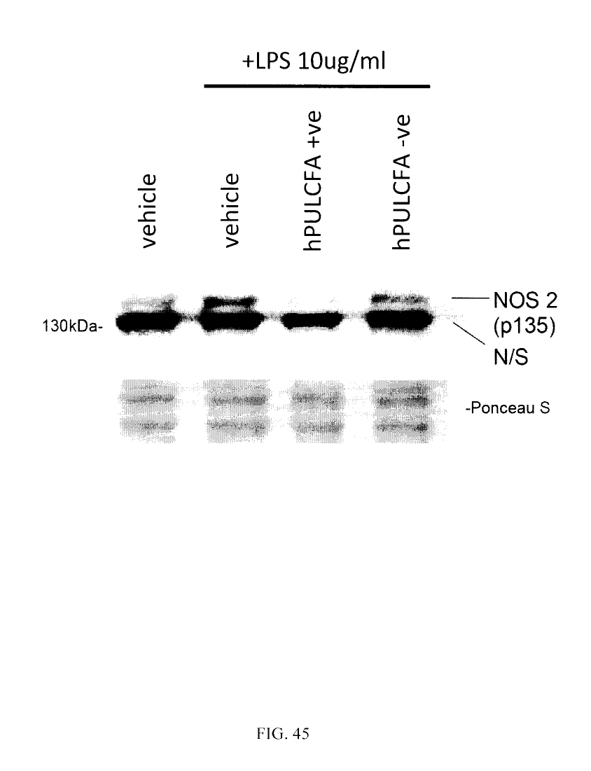

Two inducible pro-inflammatory markers were also tested to ascertain the effect hPULCFAs. First, TNF-alpha levels were measured following induction by LPS in RAW cells and found, as seen in the bar graph in FIG. 44, to be reduced in samples treated with hPULCFA-positive extract, but not in samples treated with hPULCFA-negative extract. This result suggests that hPULCFA-containing extracts have the ability to protect against an inflammation as assessed through TNF-alpha. Levels of the second pro-inflammatory marker, inducible nitric oxide synthase (NOS2), were measured by Western blot analysis following combined treatment with LPS and hPULCFA positive and negative fractions. As can be seen in the top pane of FIG. 45, hPULCFA-enriched extract reduces LPS-induced NOS2 in RAW cells. The bottom pane of the figure shows the corresponding Ponceau S stained gel. This result is also consistent with an anti-inflammatory role for hPULCFAs.

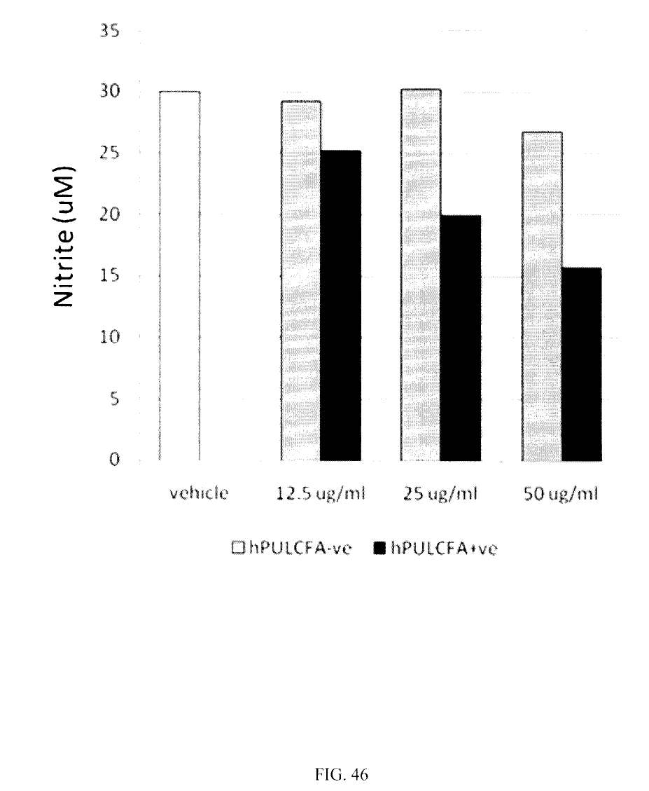

FIG. 46 shows that hPULCFA positive extracts reduce nitrite levels in conditioned media of cells in a dose dependent manner. Nitrite is a stable metabolite of nitric oxide, which can react with various organic compounds forming nitrosamines and other nitrate radicals that can be mutagenic. Nitric oxide (NO) is also produced by nitric oxide synthase, which as noted above is inhibited at the protein level (FIG. 45) by hPULCFA positive extracts. Notably, NO is induced during various inflammatory responses such as bacterial infections, and has been directly implicated as a cause of colon cancer (Erdman et al, PNAS, Jan. 27, 2009, vol 106 No. 4).

### 3. Synthesis of hPULCFA D046-124

### Structure:

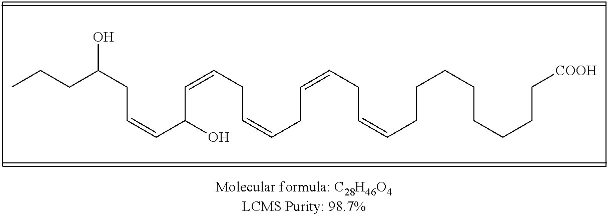

### Synthetic Scheme for Fragment A:

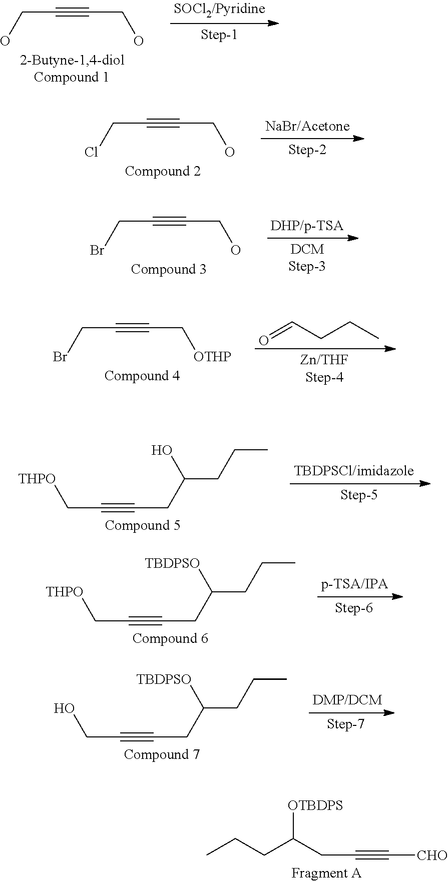

### Synthetic Scheme for Fragment B:

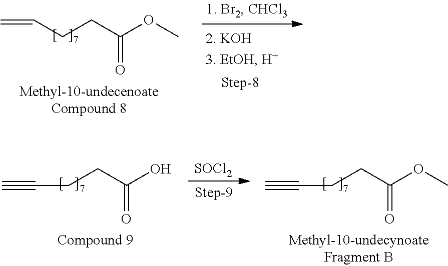

### Synthetic Scheme for Fragment C:

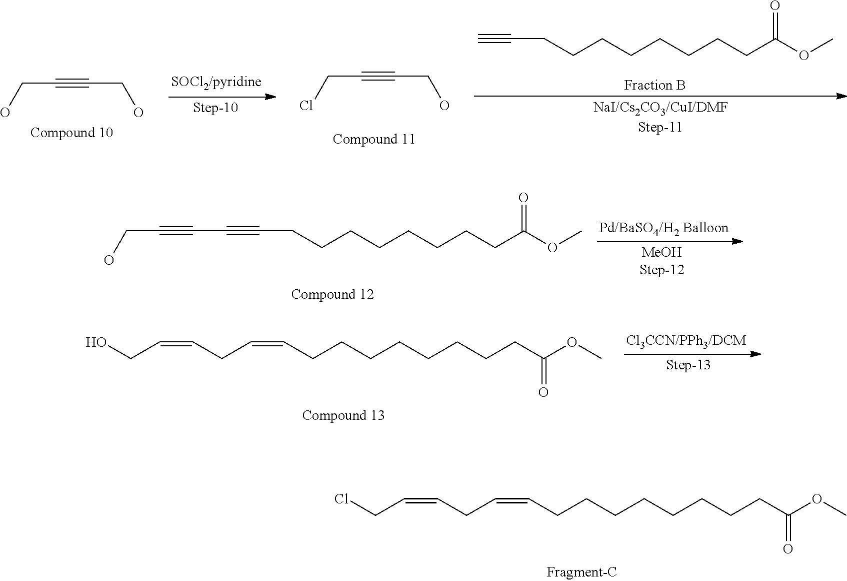

### Synthetic Scheme for D046-124 (Also Referred to Herein as GVK-FFS-09-06-PHM):

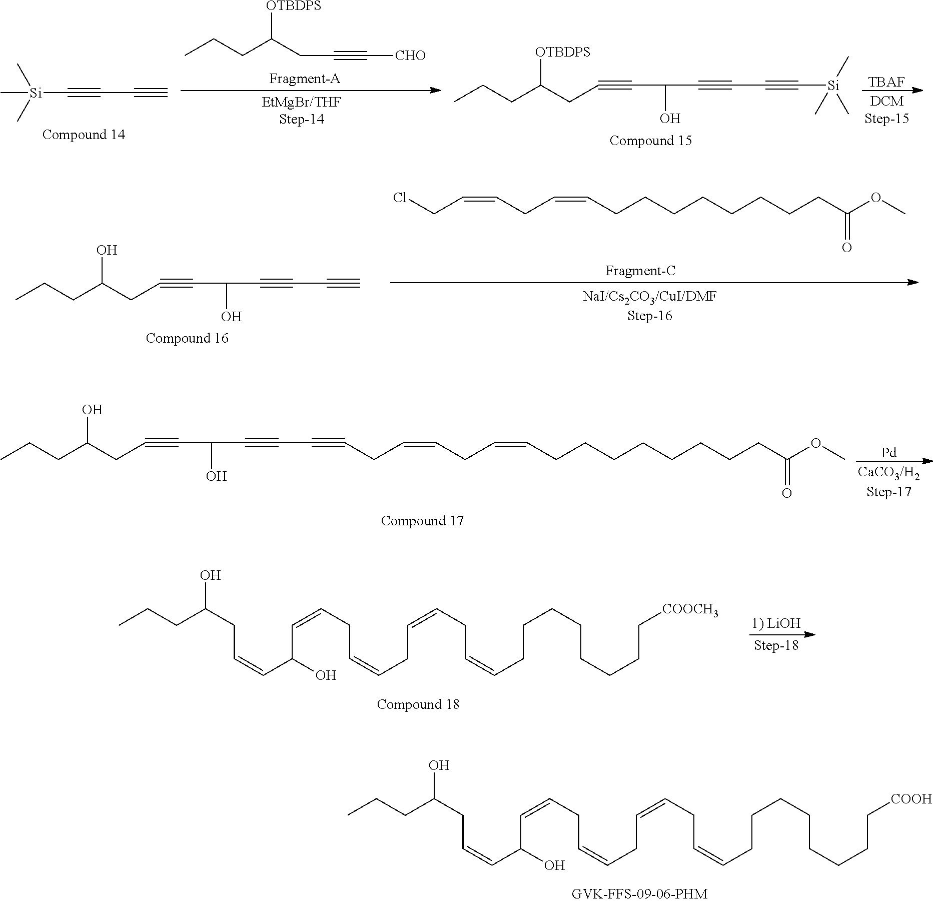

### Step 1:

### LNB Reference No: B 064-015A2

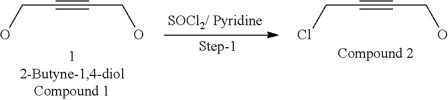

Procedure: To a solution of compound 1 (250 g 2.906 mol) in benzene (400 ml) was added pyridine (262 ml, 3.196 mol) followed by SOCl₂ (225 ml, 3.196 mol) at 0° C. and stirred overnight at room temperature until the starting material disappeared on TLC (solvent system 20% EtOAc in pet ether, product R_{f}=0.5).

Work up: On completion of the reaction, the reaction mixture was quenched with ice cold water extracted with EtOAc (200 ml×3) and the combined organic layers were washed with NaHCO₃ solution, water (300 ml×2) and brine (200 ml) and dried over anhyd Na₂SO₄ and concentrated under reduced pressure to afford compound 2 (100 g) in 30% yield as a light brown oil (B 064-015A2).

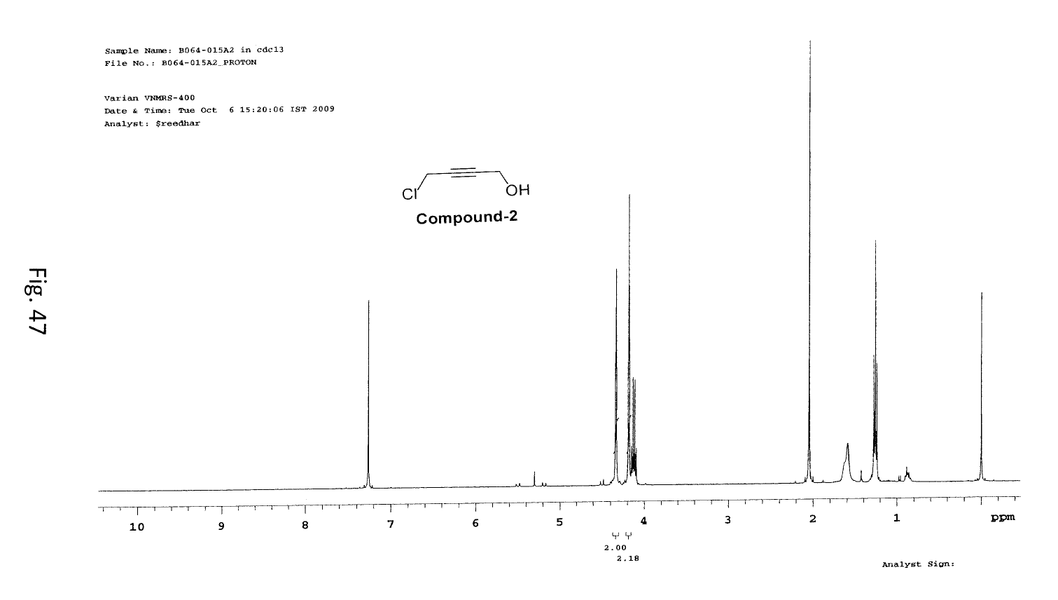

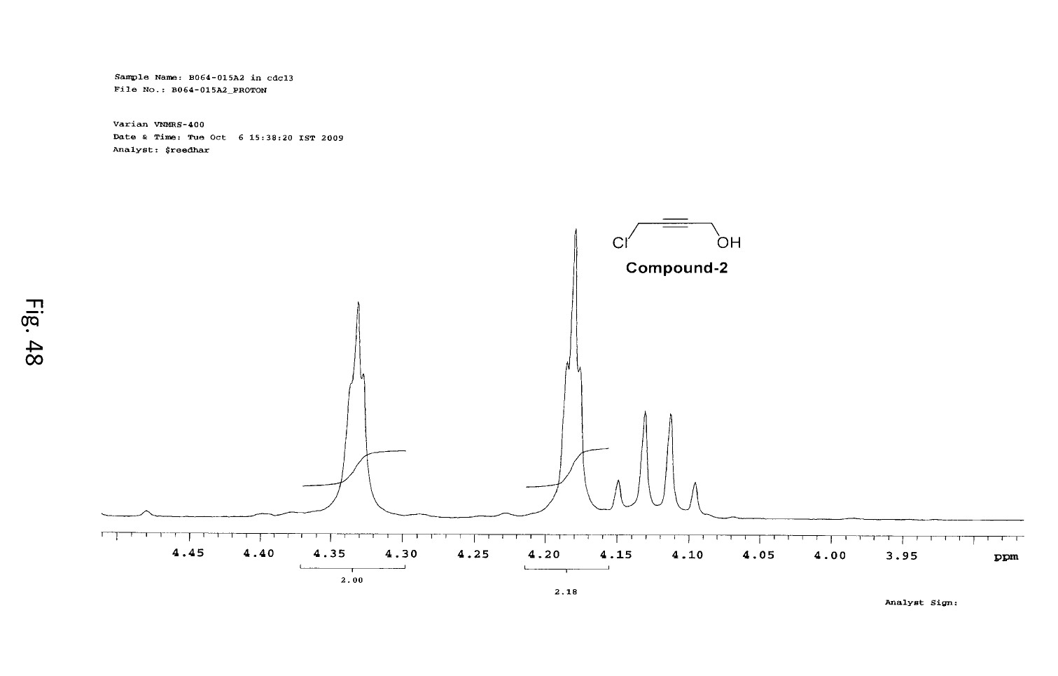

Characterization: ¹H NMR (400 MHz, CDCl₃) δ: 4.18 (s, 2H), 4.34 (s, 2H) (FIGS. 47 and 48).

### Step 2:

### LNB Reference No: B 064-017A2

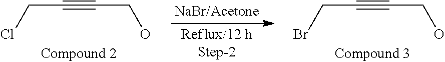

Procedure: Compound 2 (5 g, 48.07 mmol) in acetone (50 ml) was added NaBr (7.3 g, 72.11 mmol) and refluxed overnight until the starting material disappeared on TLC. (Solvent system 20% EtOAc in pet ether, product R_{f}=0.3).

Work up: On completion of the reaction, solvent was concentrated under reduced pressure and diluted with DCM (50 ml) washed with water (50 ml×2) and brine (50 ml) dried over anhyd Na₂SO₄ and concentrated under reduced pressure to obtain Compound 3 (4 g) in 70% yield as a colorless oil (B 064-017A2).

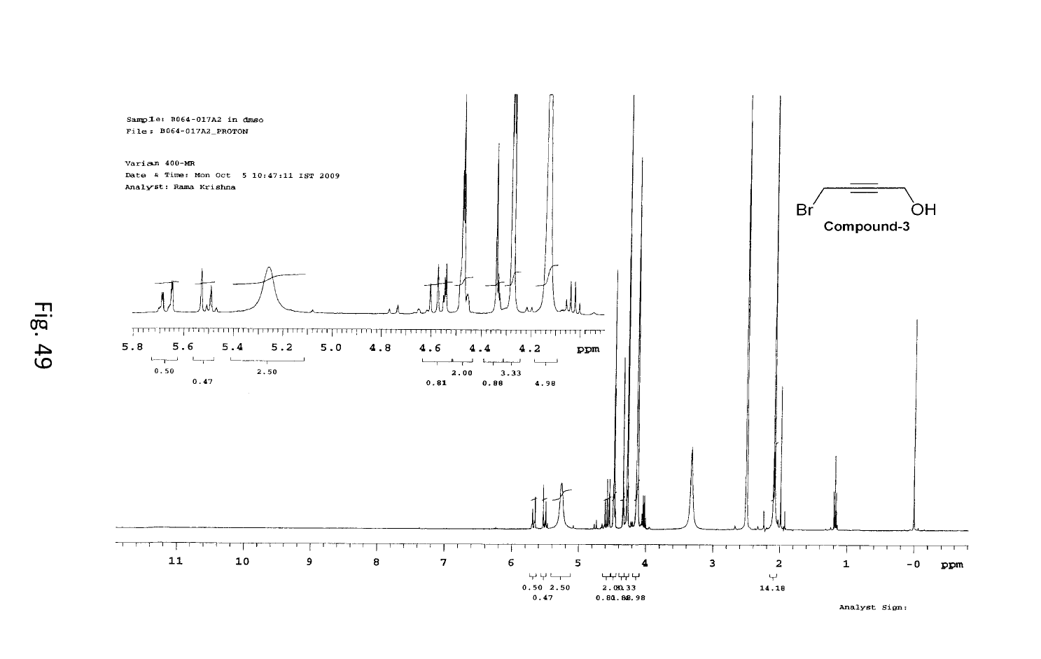

Characterization: ¹H NMR (400 MHz, DMSO) δ: 4.3 (s, 2H), 4.45 (s, 2H) (FIG. 49).

### Step 3:

### LNB Reference No: B 064-025A1

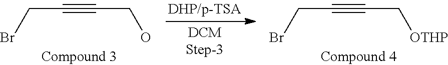

Procedure: Compound 3 (150 g, 1.00 mol) in DCM (1.5 lit) was added pTSA (1.9 g, 10.06 mmol) at 0° C. followed by DHP (91 ml, 1.006 mol) drop wise and stirred for overnight at RT until the starting material disappeared on TLC. (Solvent system 20% EtOAc in pet ether, product R_{f}=0.8).

Work up: On completion of the reaction, reaction mix was diluted with DCM (500 ml) washed with water (500 ml×2) and brine (500 ml) dried over anhyd Na₂SO₄ and concentrated under reduced pressure to obtain crude compound, which was further purified by Silica gel (100-200 mesh) column chromatography to afford compound 4 (234.5 g) in 85% yield as a colorless oil (B 064-025A1).

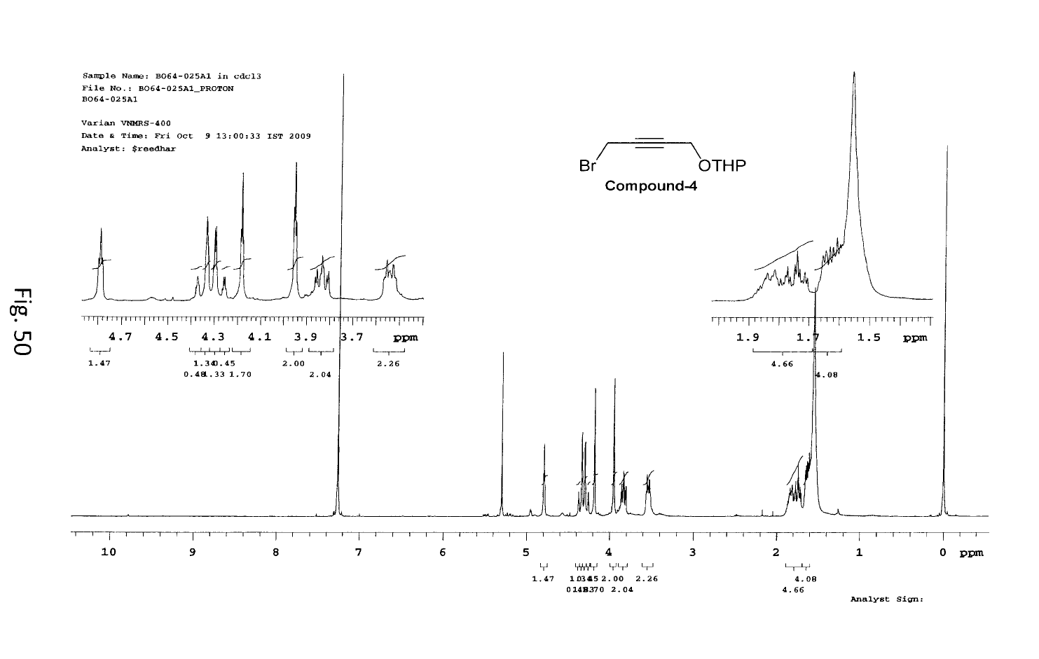

Characterization: ¹H NMR (400 MHz, CDCl₃) δ: 1.5-1.79 (m, 4H), 1.8-1.9 (m. 4H), 4.0 (m, 2H), 4.2 (m, 2H), 4.8 (m, 1H) (FIG. 50).

### Step 4:

### LNB Reference No: B 064-034A2

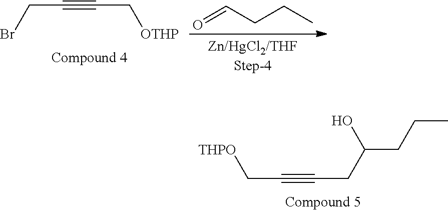

Procedure: To a suspension of zinc (139 g, 2.145 mol) in THF (500 ml) was added HgCl₂ (30 mg) and stirred for 10 min, then compound 4 (200 g, 0.858 mol) was added followed by butyraldehyde (92 ml, 1.030 mol) in THF (1 lit) under reflux condition then stirring was continued at RT overnight until the starting material disappeared on TLC. (Solvent system 20% EtOAc in pet ether, product R_{f}=0.8).

Work up: On completion of the reaction, the reaction mixture was quenched with AcOH and compound extracted with EtOAc (500 ml×2) washed with water (500 ml×2) and brine (500 ml) dried over anhyd Na₂SO₄ and concentrated under reduced pressure to obtain crude compound which was further purified by Silica gel (100-200 size) column chromatography to afford compound 5 (35 g) in 25% yield as a light yellow oil (B 064-034A2).

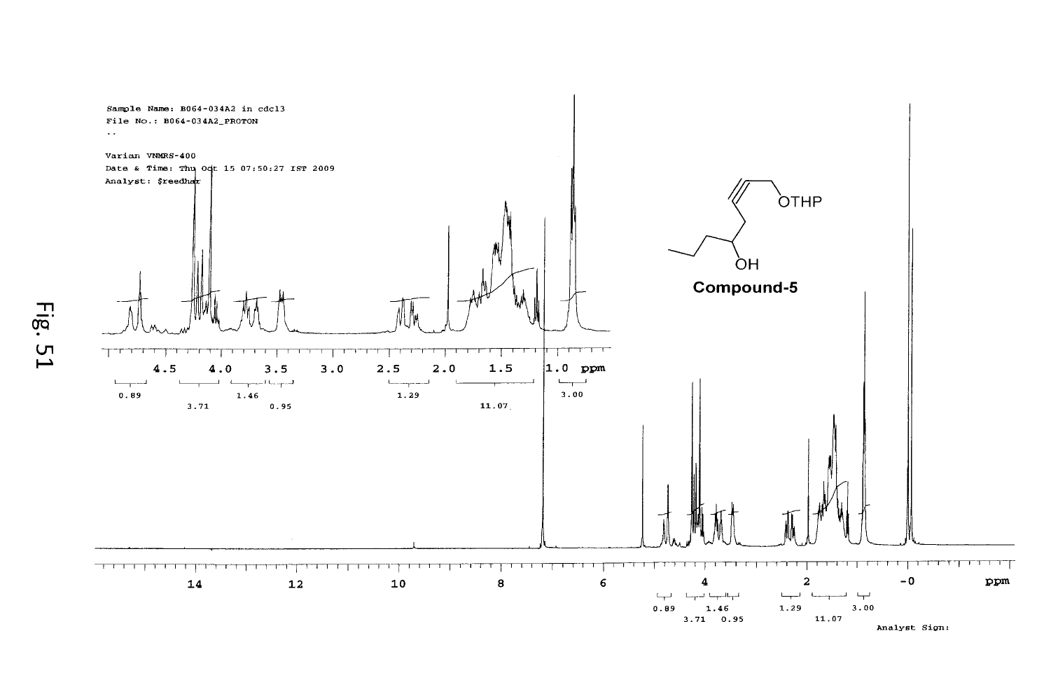

Characterization: ¹H NMR (400 MHz, CDCl₃) δ: 0.9 (s, 3H), 1.2-1.8 (m, 11H), 3.5 (m, 1H), 3.7-3.9 (m, 2H), 4.0-4.3 (m, 4H) (FIG. 51).

### Step 5:

### LNB Reference No: B 064-042A1

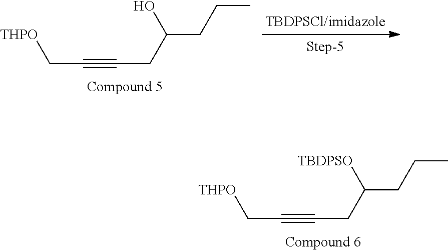

Procedure: To a suspension of compound 5 (35 g, 15.486 mmol) in DCM (350 ml) was added Imidazole (26 g, 38.71 mmol) followed by tert-Butyl Diphenylchlorosilane (TBDPSCl) (43 ml, 17.035 mmol) at 0° C. and stirred for overnight at RT until the starting material disappeared on TLC. (Solvent system 20% EtOAc in pet ether, product R_{f}=0.8).

Work up: On completion of the reaction, the reaction mixture was diluted with DCM (100 ml×2) washed with water (200 ml×2) and brine (100 ml) dried over anhyd Na₂SO₄ and concentrated under reduced pressure to afford compound 6 (75 g, 85%) as a light yellow liquid (B 064-042A1).

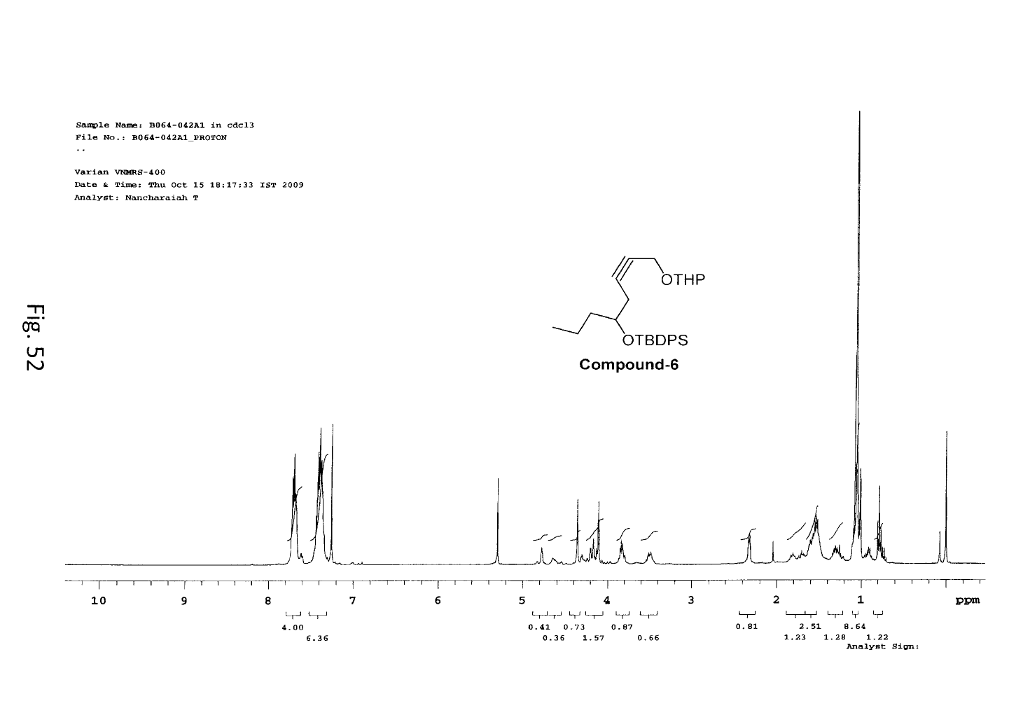

Characterization: ¹H NMR (400 MHz, CDCl₃) δ: 0.9 (t, 3H), 1.1 (m, 17H), 1.3 (m, 1H), 1.5 (m, 2H), 1.8 (m, 1H), 2.3 (m, 1H), 3.8 (m, 2H), 4.2 (m, 2H), 4.4 (s, 1H), 7.4 (m, 6H), 7.7 (m, 4H) (FIG. 52).

### Step 6:

### LNB Reference No: B 064-049A2

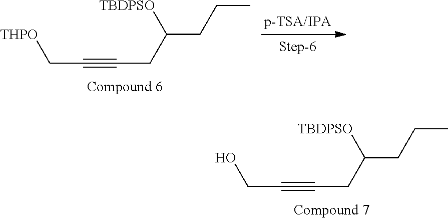

Procedure: To a suspension of compound 6 (75 g, 161.63 mmol) in 2-propanol (2.5 lit) and diethyl ether (1.25 lit) was added pTSA (3 g, 16.163 mmol) and stirred for 72 h at RT until the starting material disappeared on TLC. (Solvent system 10% EtOAc in pet ether, product R_{f}=0.4).

Work up: On completion of the reaction the reaction mixture was quenched with NaHCO₃ solution and concentrated. The residue was extracted with DCM (300 ml×2), washed with water (200 ml×2) and brine (100 ml), dried over anhyd Na₂SO₄, concentrated under reduced pressure and purified on silica gel (100-200 mesh) chromatography to afford compound 7 (27 g, 43%) as a light yellow liquid (B 064-049A2).

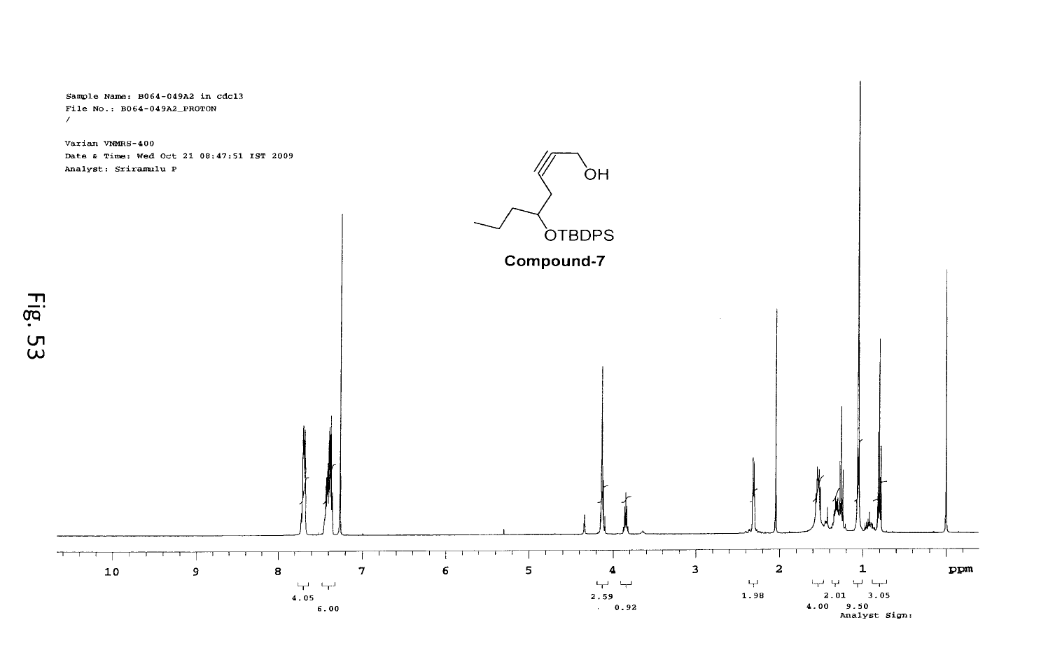

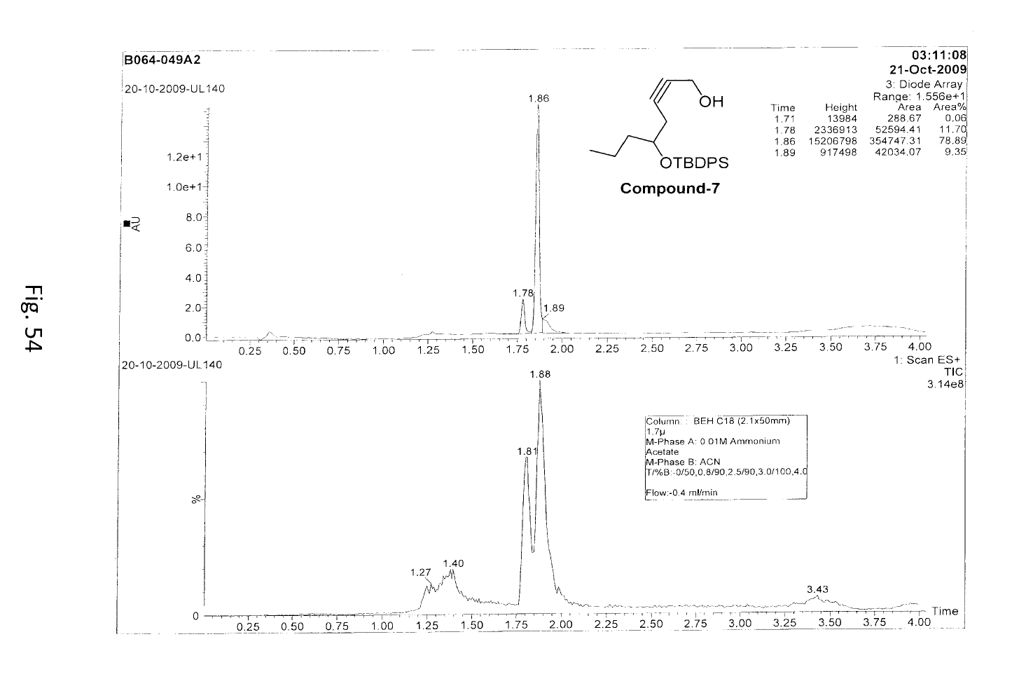

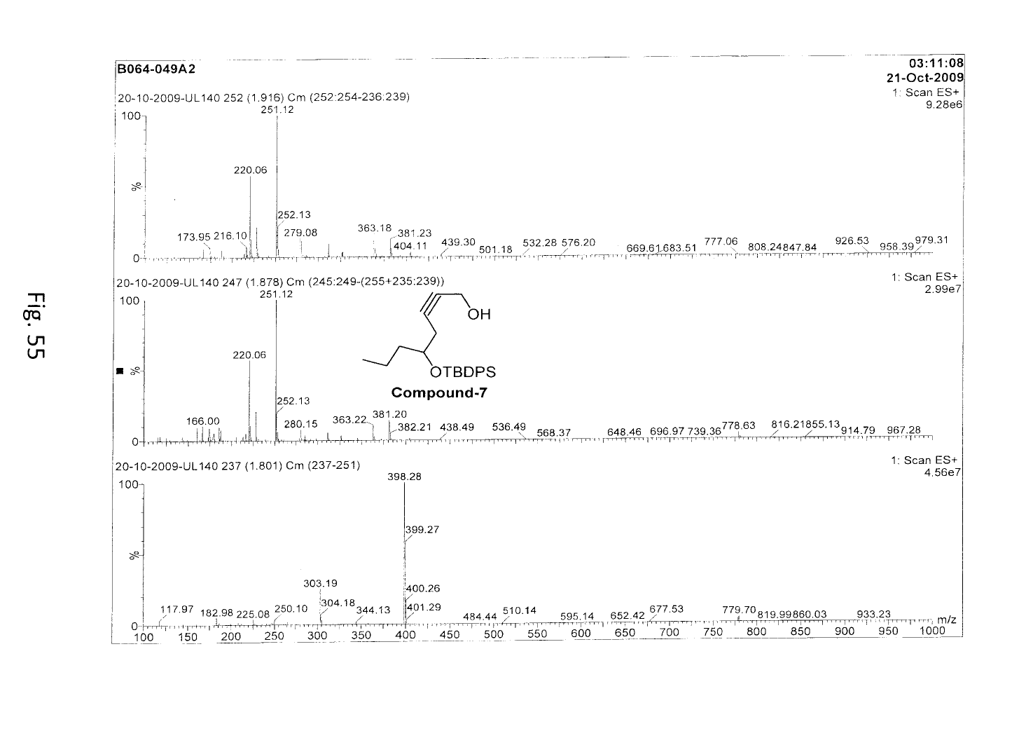

Characterization: ¹H NMR (400 MHz, CDCl₃) δ: 0.8 (t, 3H), 1.1 (s, 9H), 1.3 (m, 2H), 1.6 (m, 2H), 2.3 (m, 2H), 3.9 (m, 1H), 4.2 (m, 2H), 7.4 (m, 6H), 7.7 (m, 4H). LCMS: 79% purity, m/z=251 (m+1). (FIGS. 53-55).

### Step 7:

### LNB Reference No: B 064-051A2

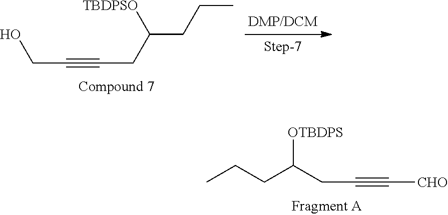

Procedure: To a suspension of compound 7 (12 g, 31.578 mmol) in DCM (100 ml) was added DMP (16 g, 34.736 mmol) at 0° C. and stirred for 30 min at RT until the starting material disappeared on TLC. (Solvent system 10% EtOAc pet ether, product R_{f}=0.5).

Work up: On completion of the reaction, the reaction mixture was diluted with DCM (100 ml) and washed with NaHCO₃ solution, water (200 ml×2) and brine (100 ml) dried over anhyd Na₂SO₄ and concentrated under reduced pressure then purified by silica gel (100-200 mesh) chromatography to afford Fragment A (8 g, 75%) as a light yellow liquid (B 064-051A2)

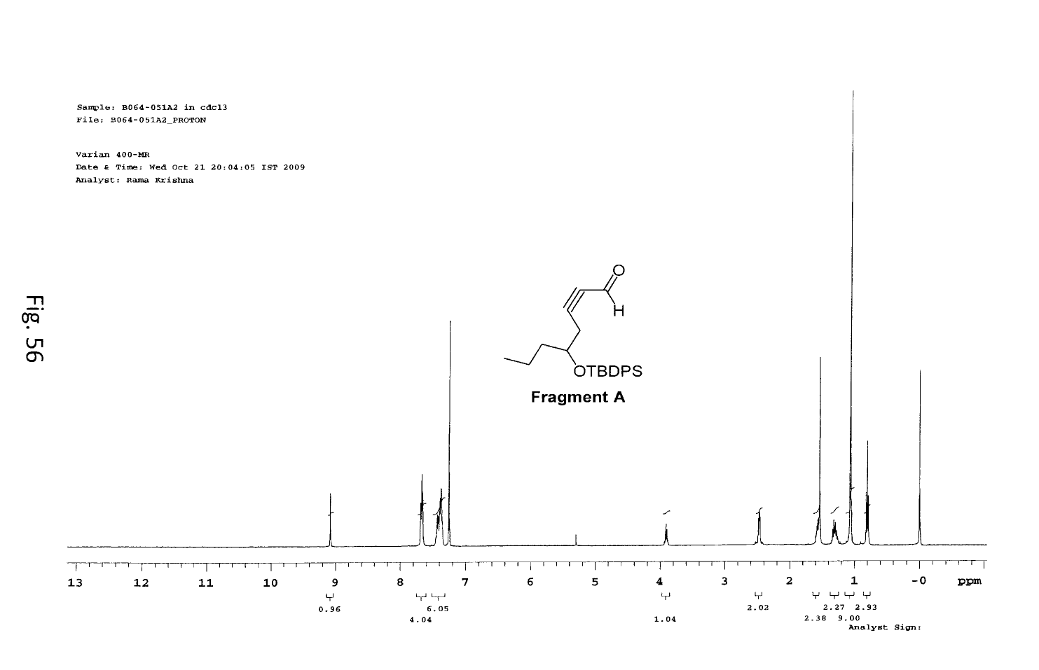

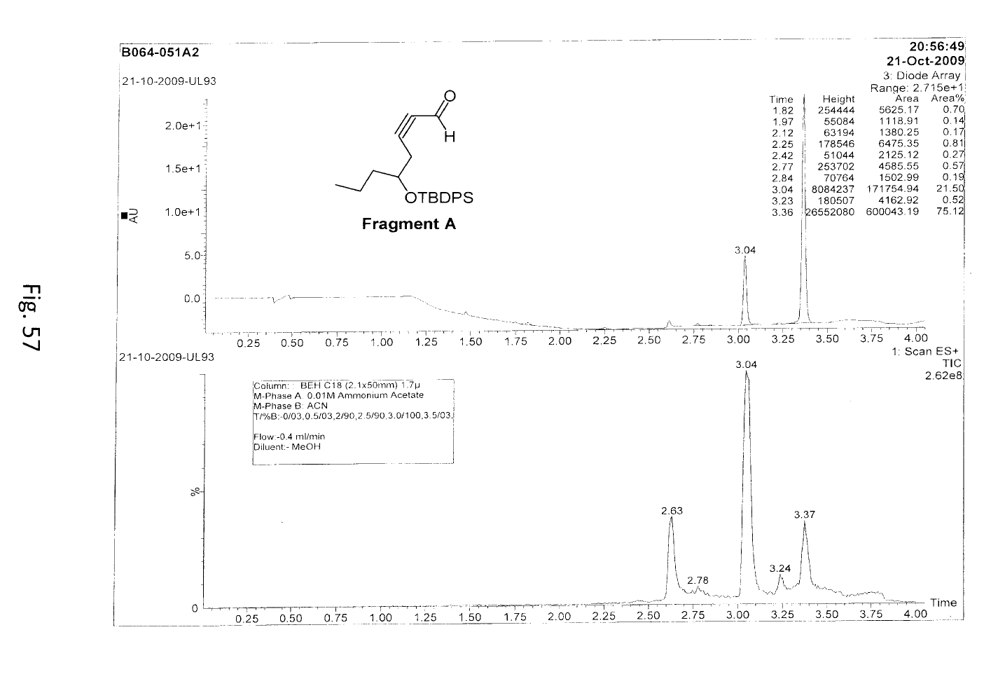

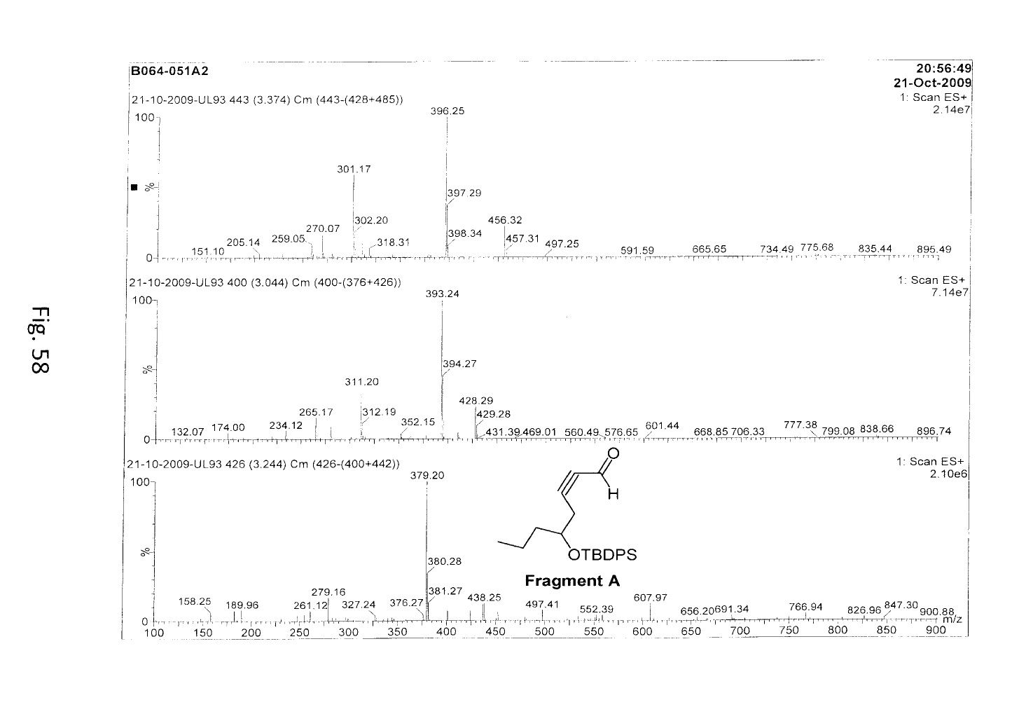

Characterization: ¹H NMR (400 MHz, CDCl₃) δ: 0.8 (t, 3H), 1.1 (s, 9H), 1.3 (m, 2H), 1.6 (m, 2H), 2.5 (m, 2H), 3.9 (m, 1H), 7.4 (m, 6H), 7.7 (m, 4H), 9.1 (s, 1H) LCMS: 75% purity, m/z=379 (m+1) (FIGS. 56-58).

### Step 8:

### LNB Reference No: GK-PHM-030A1

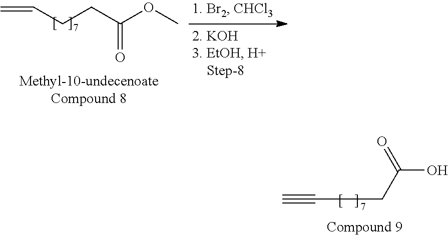

Procedure: To a stirred solution of compound 8 (25 g, 126.26 mmol) in chloroform (100 ml) was added bromine (30 ml, 631 mmol) dropwise and stirred for 2 h at RT. Then it was concentrated under reduced pressure and the residue was dissolved in ethanol (200 ml), added KOH (90 g) and stirred for 3 h at 80° C. until the starting material disappeared on TLC (20% EtOAc in pet ether R=0.6).

Work up: The reaction mixture was concentrated under reduced pressure, obtained crude was acidified with 6N HCl (30 ml), and extracted with ethyl acetate (350 ml). The organic layer was washed with water (100 ml) and brine (100 ml), dried over anhyd Na₂SO₄ and evaporated under reduced pressure to afford compound 9 (18 g, 78%) as a light brown oil (GK-PHM-030A1). It was directly used in the next step without any further purification.

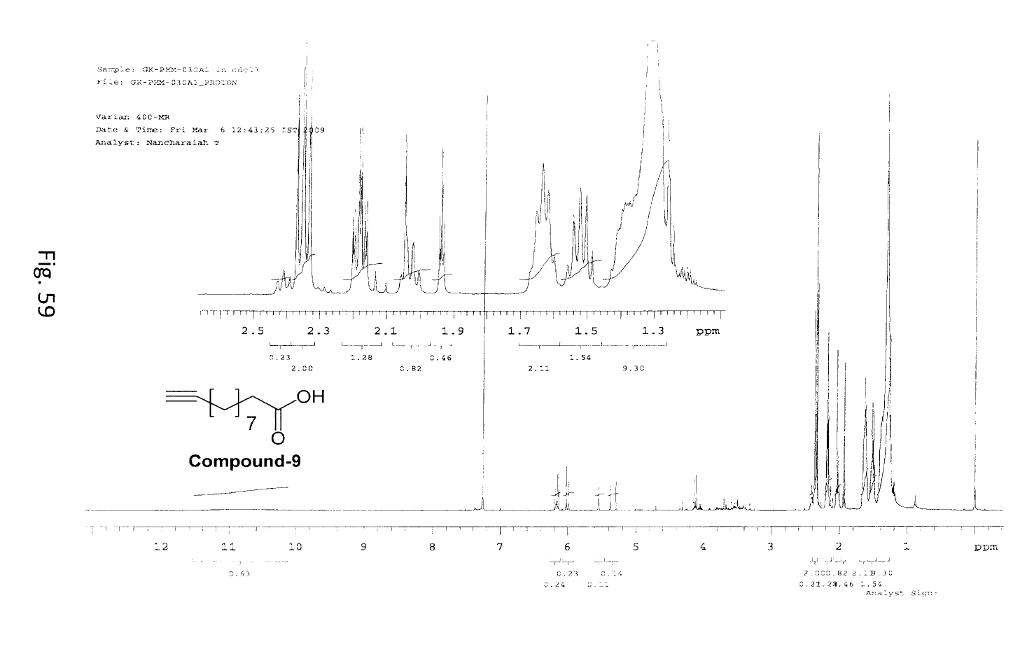

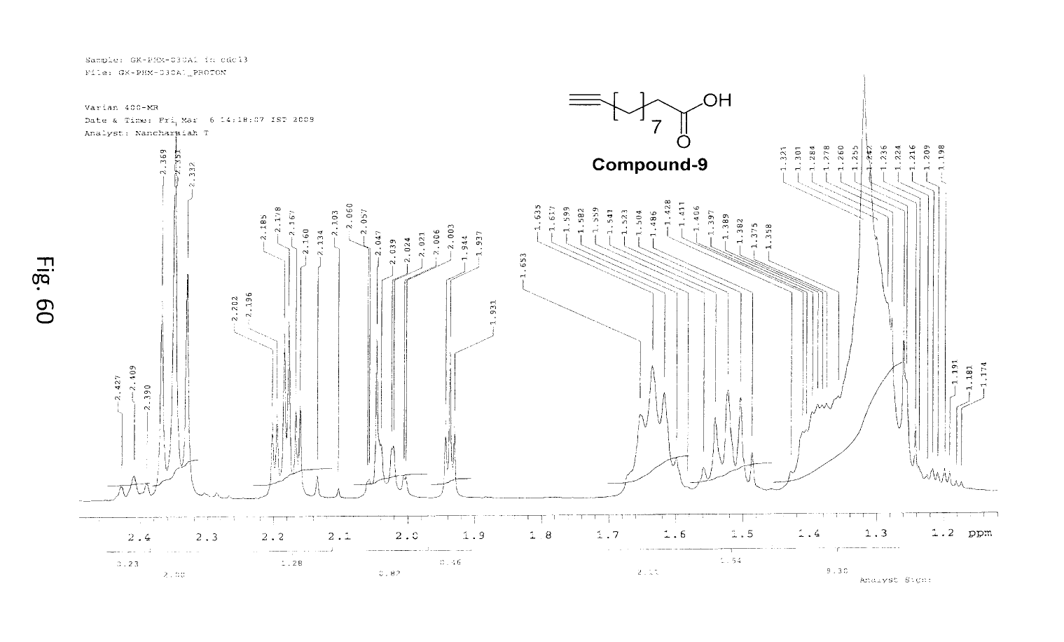

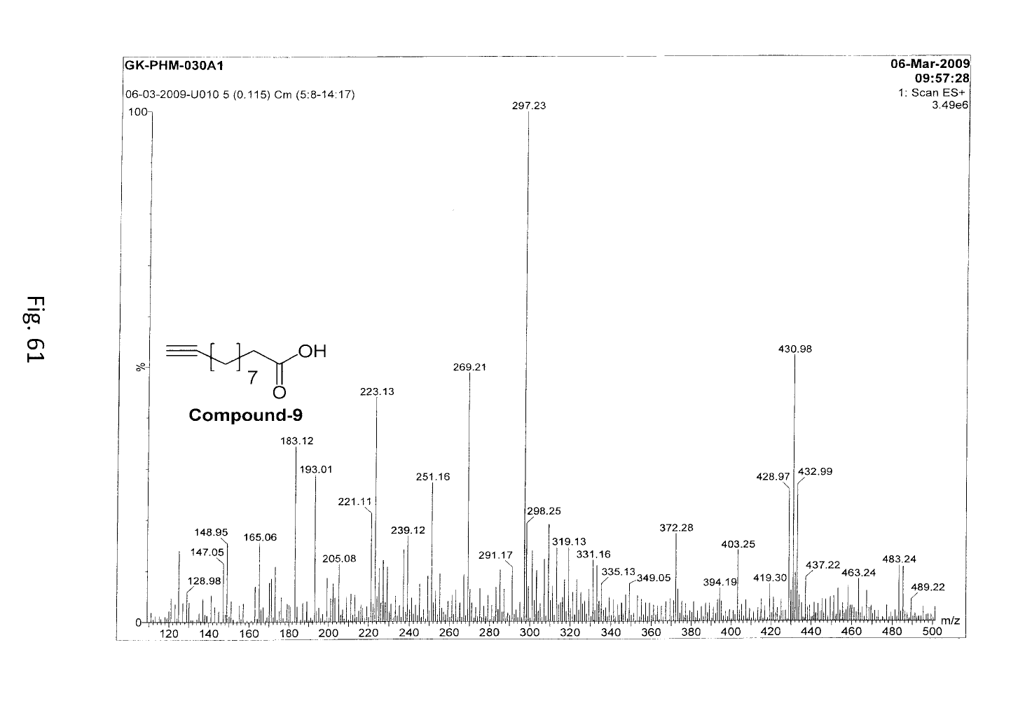

Characterization: ¹H NMR (400 MHz, CDCl₃) δ: 1.3 (m, 9H), 1.5 (m, 2H), 1.6-1.7 (m, 2H), 2.0 (m, 1H), 2.2 (m, 1H), 2.4 (m, 2H). Mass: m/z=183 (m+1) (FIGS. 59-61).

### Step 9:

### LNB Reference No: GK-PHM-032A2

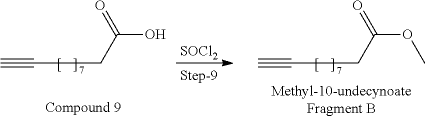

Procedure: To a stirred solution of compound 9 (18 g, 98.9 mmol) in methanol (200 ml) was added SOCl₂ (23.5 g, 197.8 mmol) drop wise at 0° C. then refluxed for overnight until the starting material disappeared on TLC. (20% EtOAc in pet ether R_{f}: 0.7).

Work up: The reaction mixture was concentrated and extracted with DCM (200 ml) then washed with NaHCO₃ solution, water (200 ml×2) and brine (100 ml) then dried over anh.Na₂SO₄ and concentrated under reduced pressure to get crude compound which was further purified by column chromatography using silica gel (100-200 mesh) to afford Fragment B (14 g, 72%) as a light yellow oil (GK-PHM-032A2).

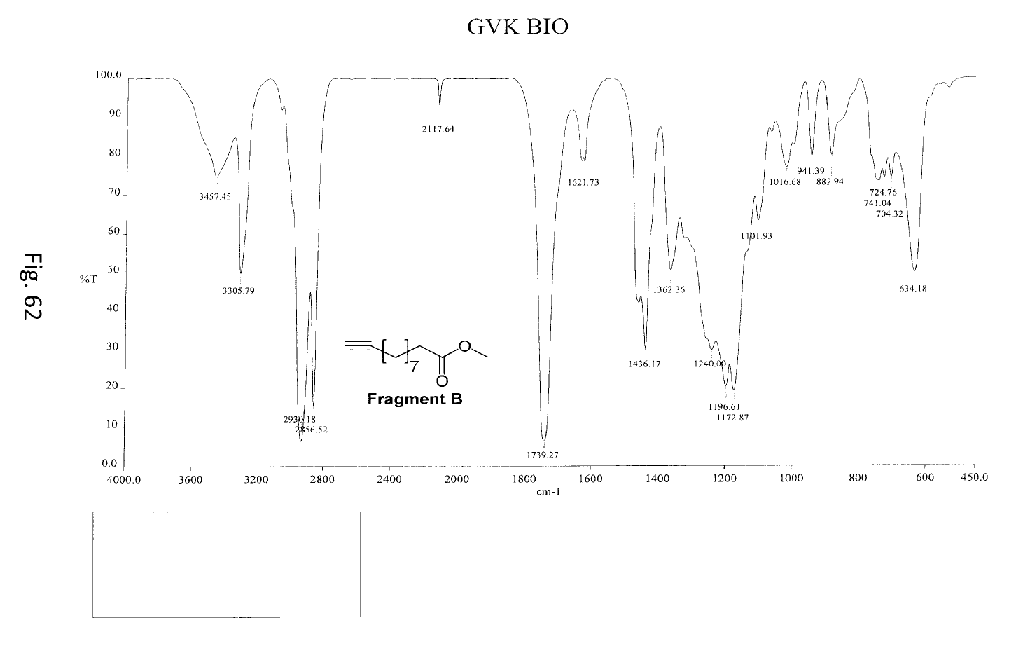

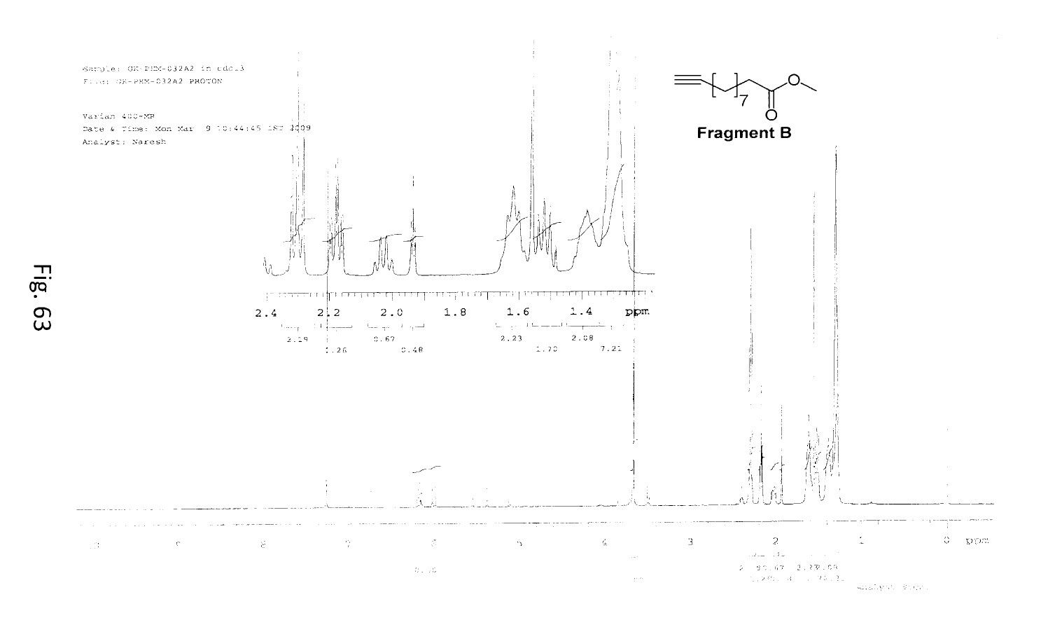

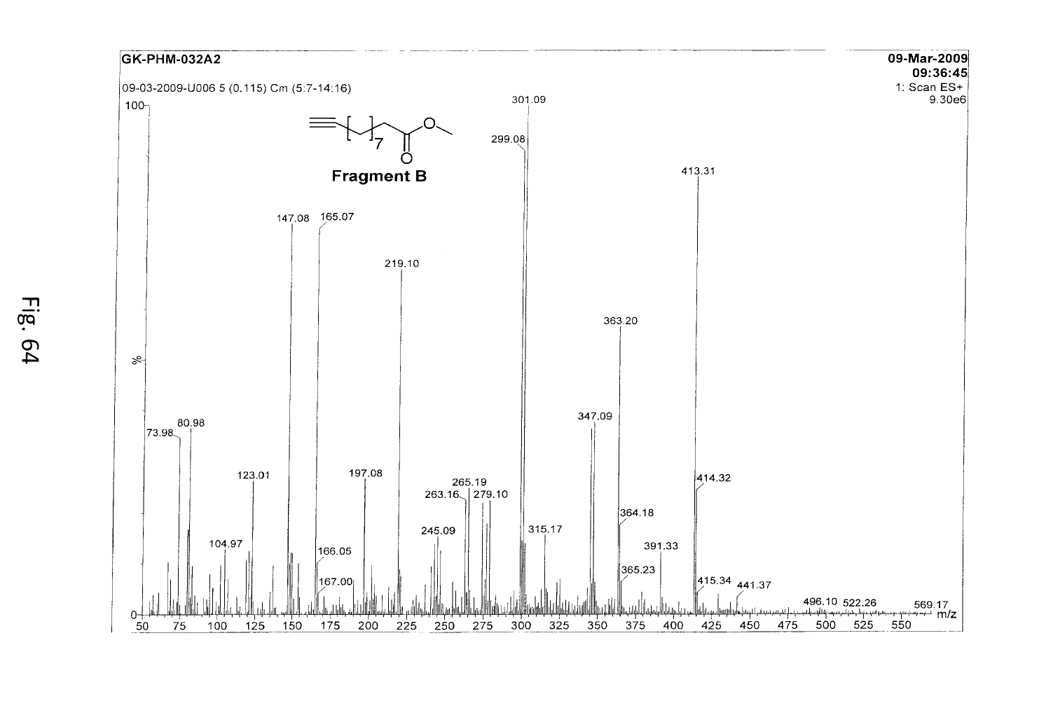

Characterization: ¹H NMR (400 MHz, CDCl₃) δ: 1.3 (m, 7H), 1.4 (m, 2H), 1.5-1.6 (m, 2H), 1.7 (m, 2H), 2.1 (m, 1H), 2.2 (m, 1H), 2.3 (m, 2H). IR (cm⁻¹): 634, 704, 741, 724, 1016, 1172, 1196, 1240, 1362, 1436, 1621, 1739, 2117, 2856, 2930, 3305, 3457. Mass: m/z=197 (m+1) (FIGS. 62-64).

### Step 10:

### LNB Reference No: B 064-015A2

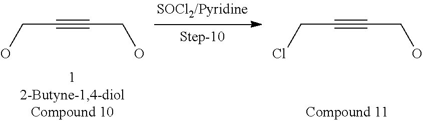

Procedure: To a solution of compound 10 (250 mg 2.906 mol) in benzene (400 ml) was added pyridine (262 ml, 3.196 mol) followed by SOCl₂ (225 ml, 3.196 mol) at 0° C. and stirred overnight at RT until the starting material disappeared on TLC (solvent system 20% EtOAc in pet ether, product R_{f}=0.5).

Work up: On completion of the reaction, the reaction mixture was quenched with ice cold water, extracted with EtOAc (200 ml×3) the combined organic layers were washed with NaHCO₃ solution, water (300 ml×2) brine (200 ml) dried over anh.Na₂SO₄ and concentrated under reduced pressure to afford compound 11 (100 g) in 30% yield.

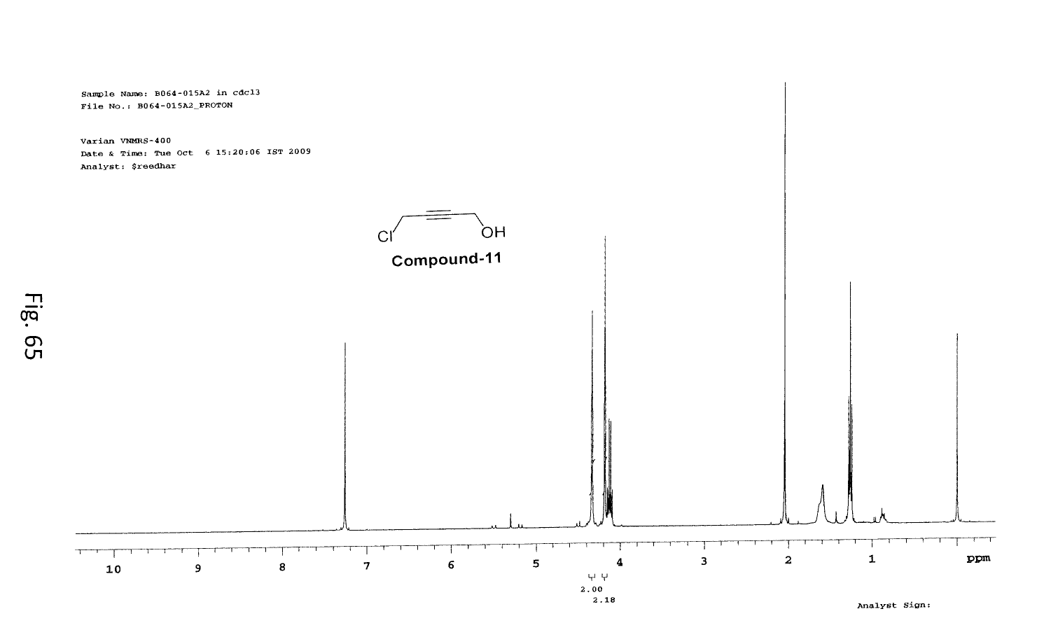

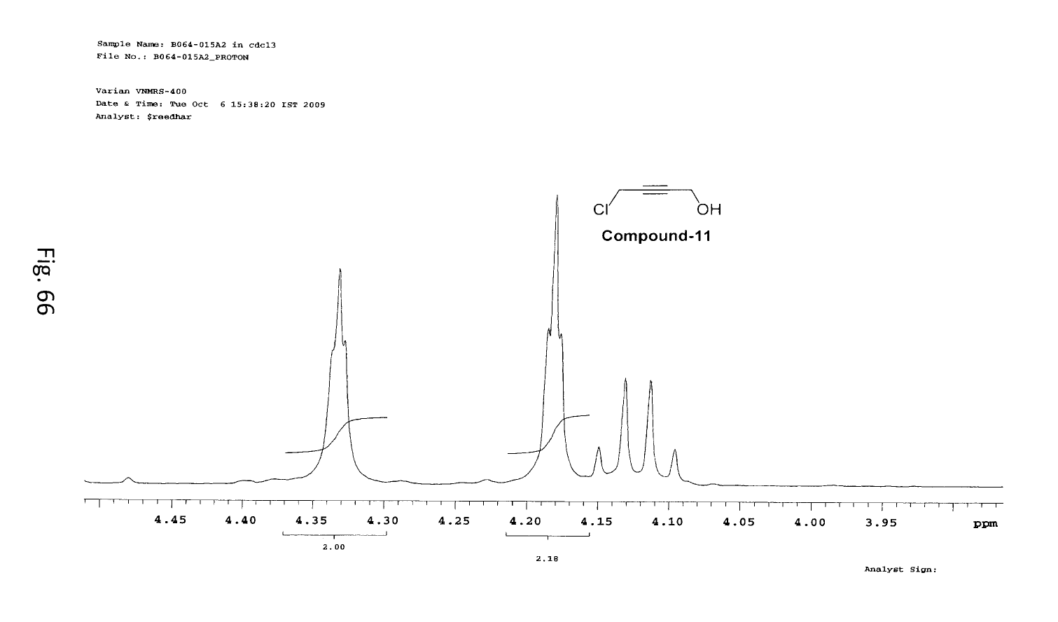

Characterization: ¹H NMR (400 MHz, CDCl₃) δ: 4.2 (S, 2H), 4.3 (s, 2H) (FIGS. 65-66).

### Step 11:

### LNB Reference No: B 064-064A2

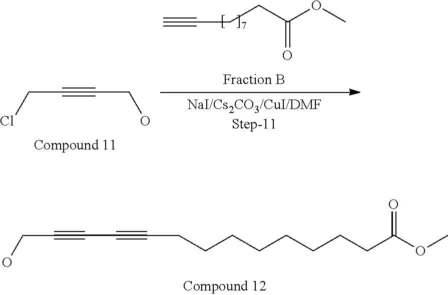

Procedure: To a solution of fraction B (7.54 g, 38.461 mmol) in dry DMF (70 ml) was added NaI (7.49 g, 49.99 mmol), Cs₂CO₃ (16.28 mmol) and CuI (9.52 g, 49.99 mmol) at 0° C. and stirred for 20 min. Then added compound 11 (4 g, 38.461 mmol) and stirred overnight at RT until the starting material disappeared on TLC (solvent system 30% EtOAc in pet ether, product R_{f}=0.3).

Work up: On completion of the reaction, the reaction mixture was quenched with NH₄Cl solution (100 ml) extracted with diethyl ether (200 ml×3) and combined organic layers were washed with water (100 ml×2) and brine (100 ml) then dried over anhyd Na₂SO₄ and concentrated under reduced pressure to afford crude compound which was further purified by silica gel (100-200 mesh) column chromatography to afford compound 12 (4.5 g, 43%) as a colorless oil (B 064-064A2).

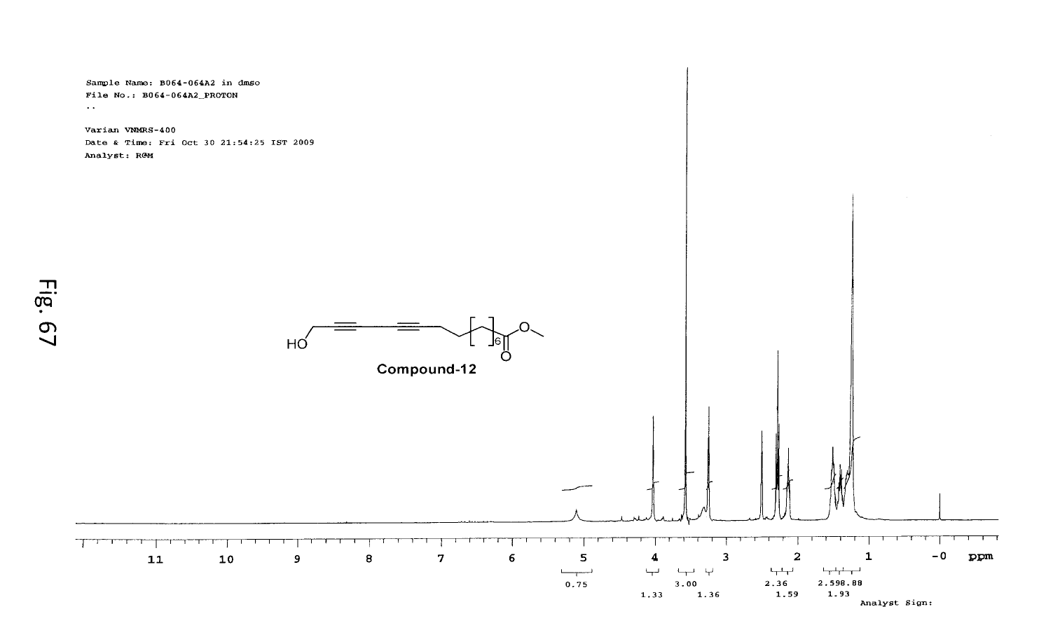

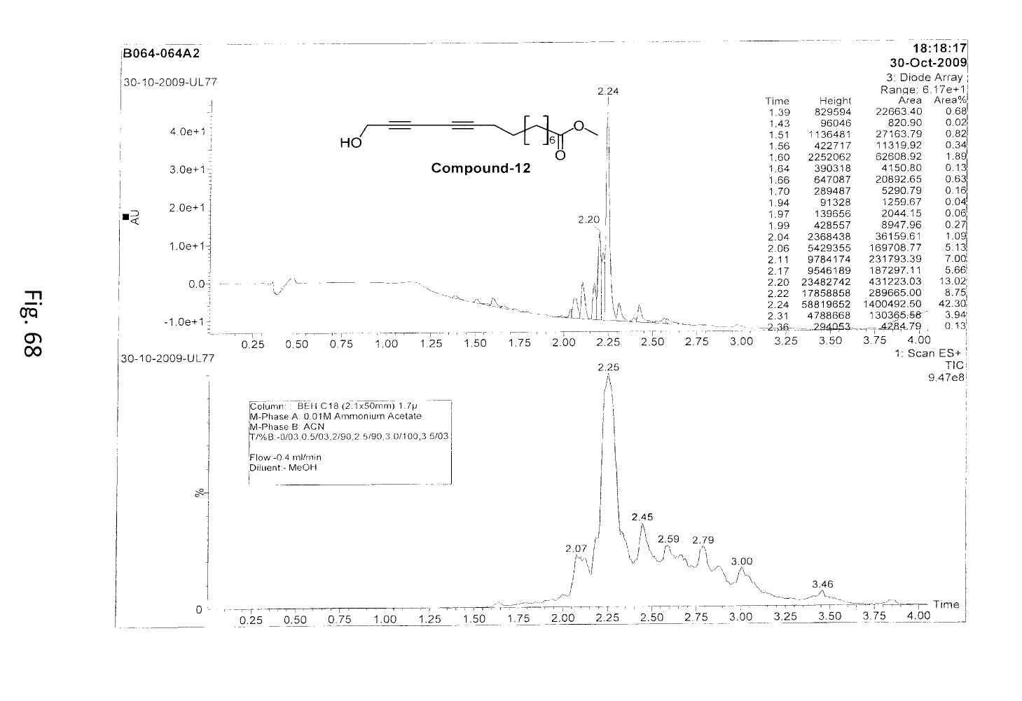

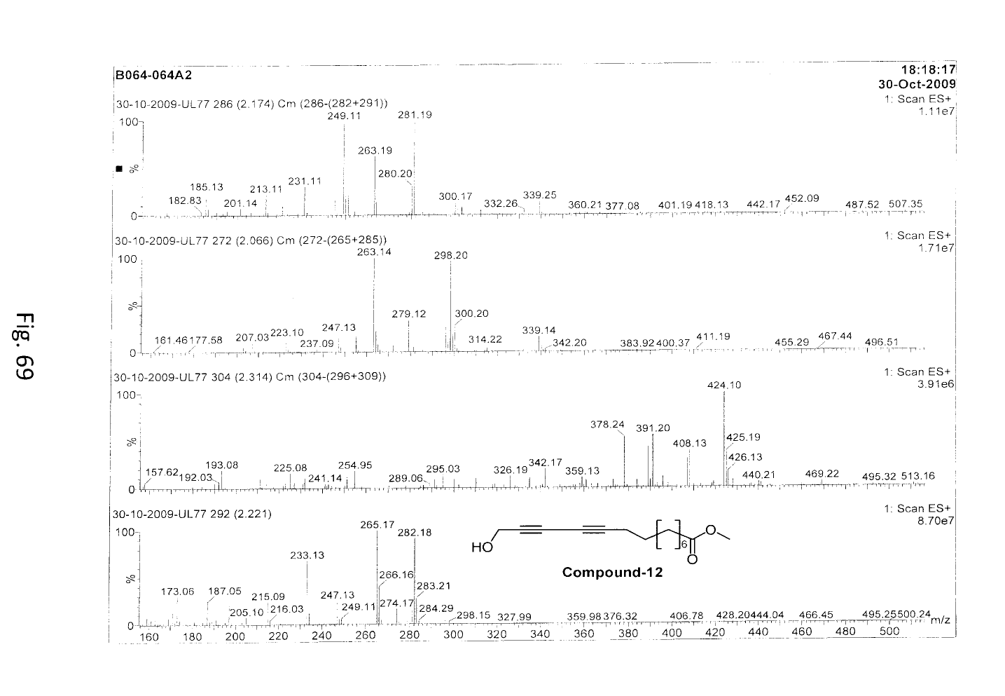

Characterization: ¹H NMR (400 MHz, DMSO) δ: 1.2 (m, 8H), 1.4 (m, 2H), 1.5 (m, 2H), 2.1 (m, 2H), 2.3 (m, 2H), 3.2 (m, 2H), 3.6 (s, 3H), 4.1 (m, 2H), 5.1 (bs, 1H). LCMS: 42% purity, m/z=265 (m+1) (FIGS. 67-69).

### Step 12:

### LNB Reference No: B 064-114A2

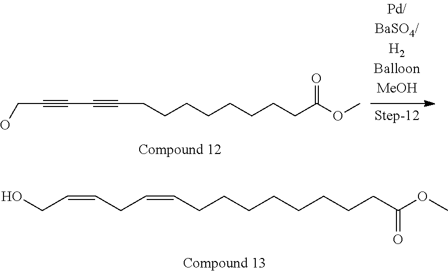

Procedure: Pd/BaSO₄ (250 mg) was added to a stirred solution of compound 12 (5 g) in dry methanol (50 ml) and stirred under H₂ pressure for 6 h at RT until the starting material disappeared on TLC (solvent system 30% EtOAc in pet ether, product R_{f}=0.4).

Work up: On completion of the reaction, the reaction mixture was filtered through celite bed and washed with methanol (20 ml×3) the filtrate was concentrated under reduced pressure to obtain crude product which was further purified by silica gel (100-200 mesh) column chromatography to afford compound 13 (3.6 g) in 68% yield as a colorless oil (B 064-114A2).

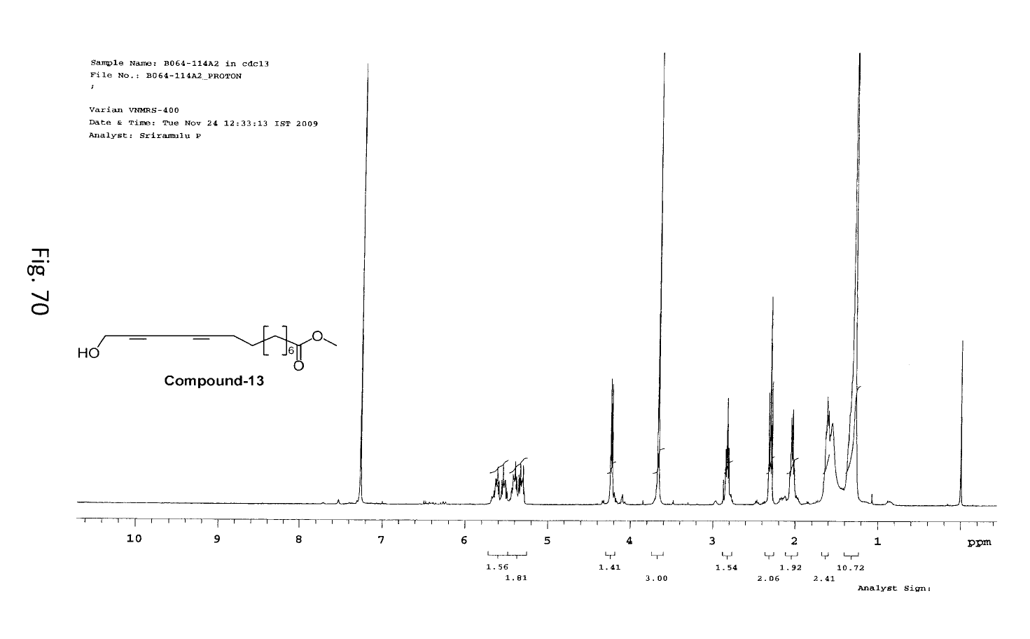

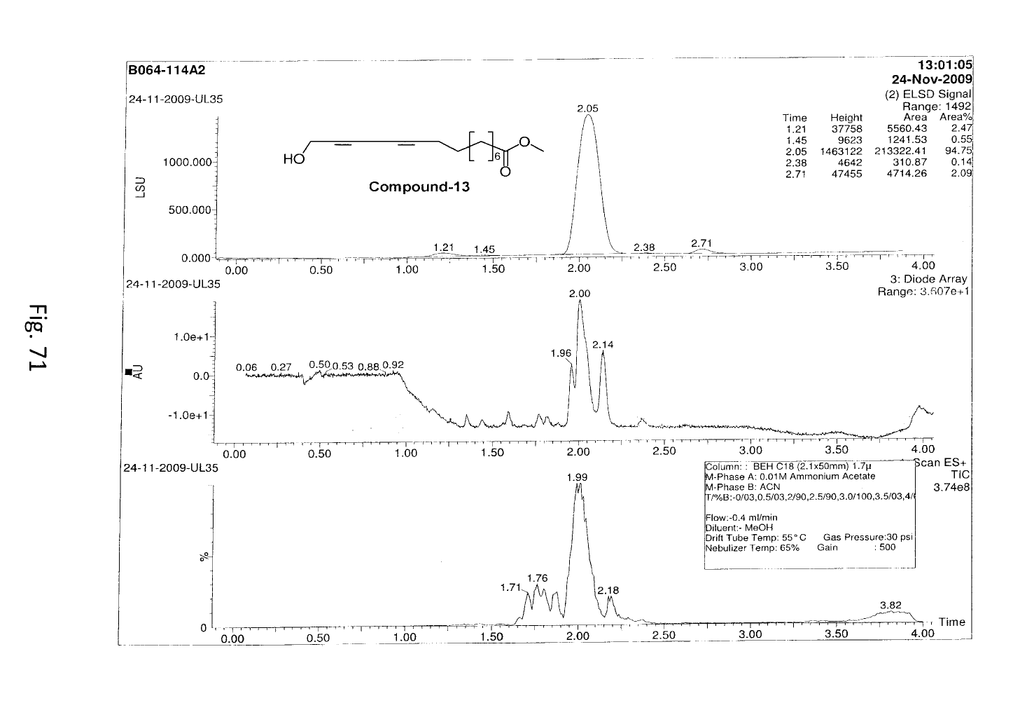

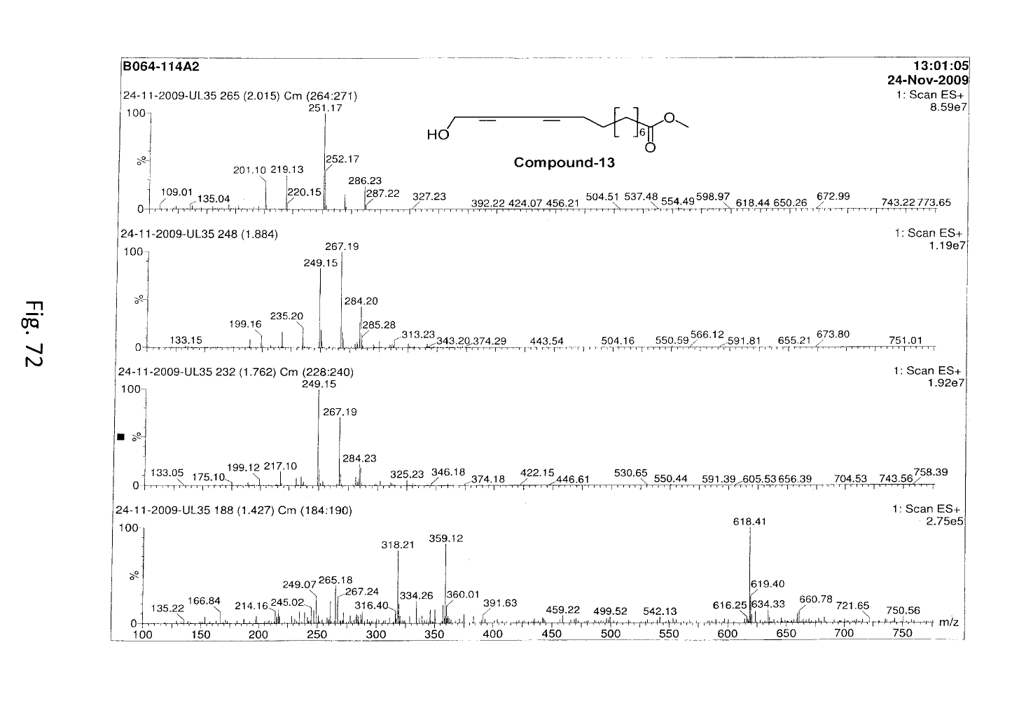

Characterization: ¹H NMR (400 MHz, CDCl₃) δ: 1.3 (m, 10H), 1.6 (m, 2H), 2.1 (m, 2H), 2.3 (m, 2H), 2.8 (m, 2H), 3.7 (s, 3H), 4.3 (m, 2H), 5.3-5.5, (m, 2H, J=7.1), 5.5-5.7 (m, 2H, J=6.4), LCMS: 94.75% purity, m/z=268 (m+1) (FIGS. 70-72).

### Step 13:

### LNB Reference No: B 064-123A2

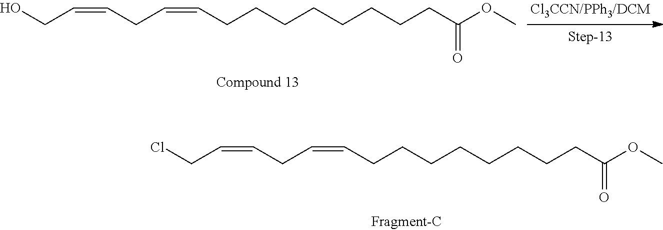

Procedure: To a stirred solution of compound 13 (4.1 g, 15.29 mmol) in DCM (60 ml) was added PPh₃ (5.21 g, 19.87 mmol) followed by trichloroacetonitrile (4.4 g, 30.59 mmol) at 0° C. and stirred for 6 h at RT until the starting material disappeared on TLC (solvent system 30% EtOAc in pet ether, product R_{f}=0.8).

Work up: On completion of the reaction, the reaction mixture was diluted with DCM (50 ml) and washed with water (100 ml×2) and brine (100 ml) then concentrated under reduced pressure to obtain crude compound which was further purified by silica gel (100-200 mesh) column chromatography to afford Fragment-C (3.1 g) in 70% yield as colorless oil (B 064-123A2).

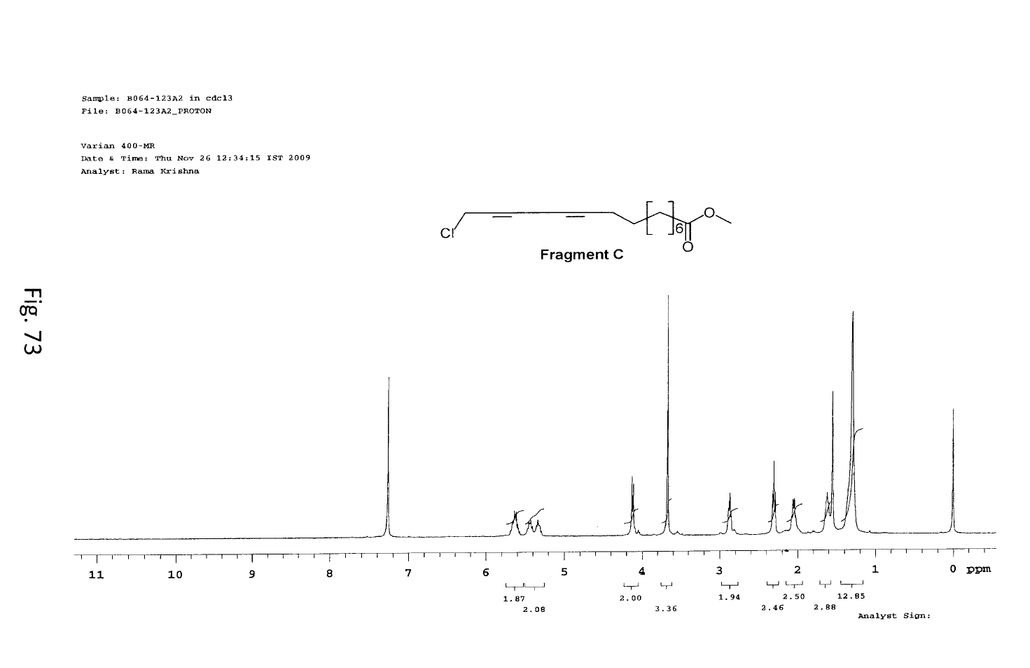

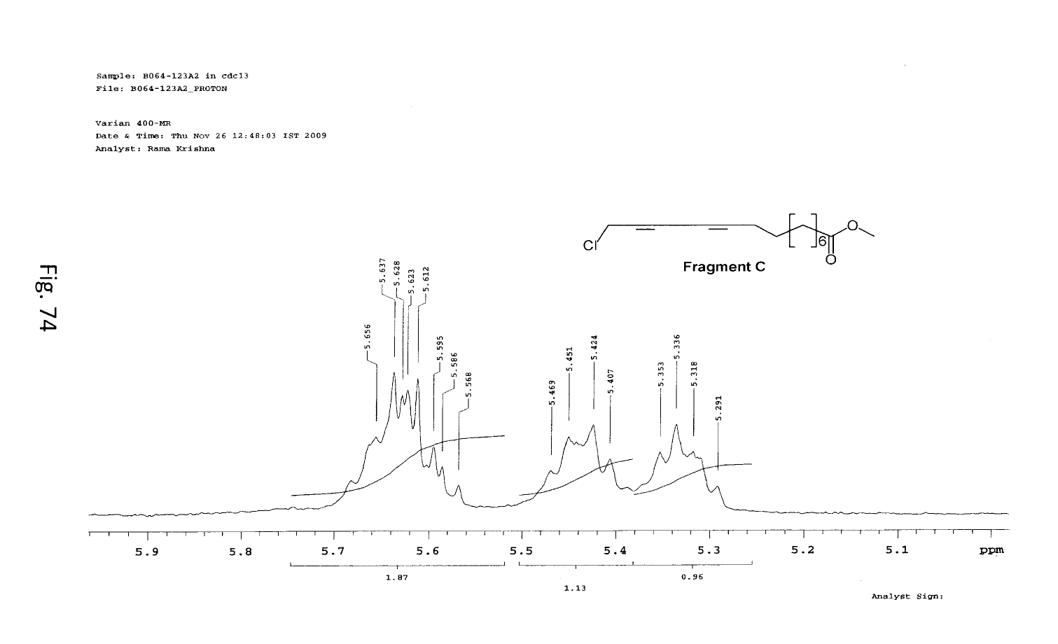

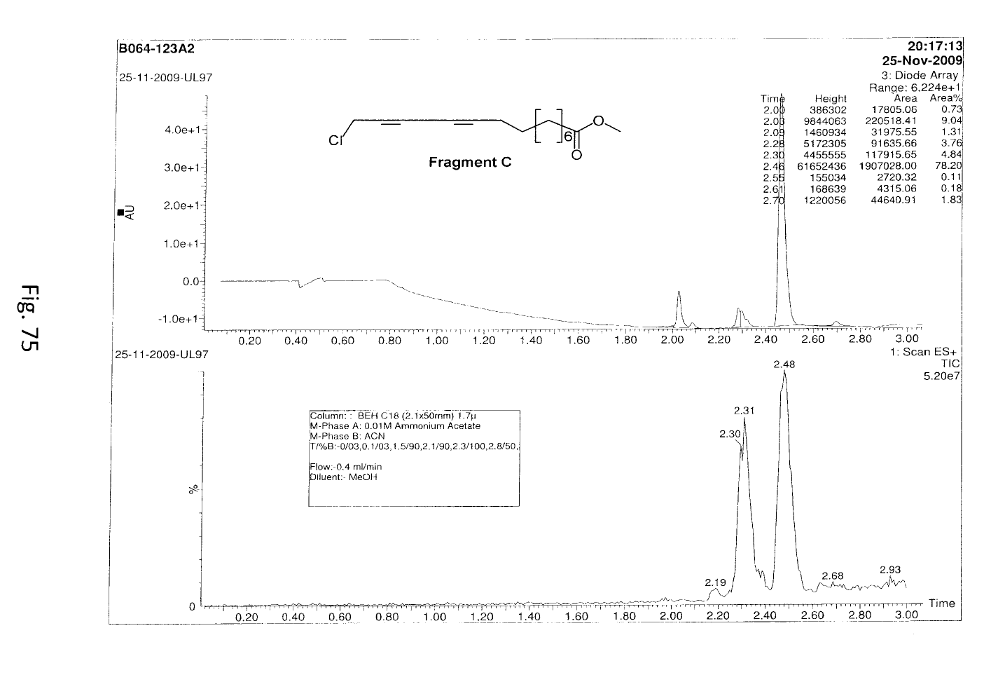

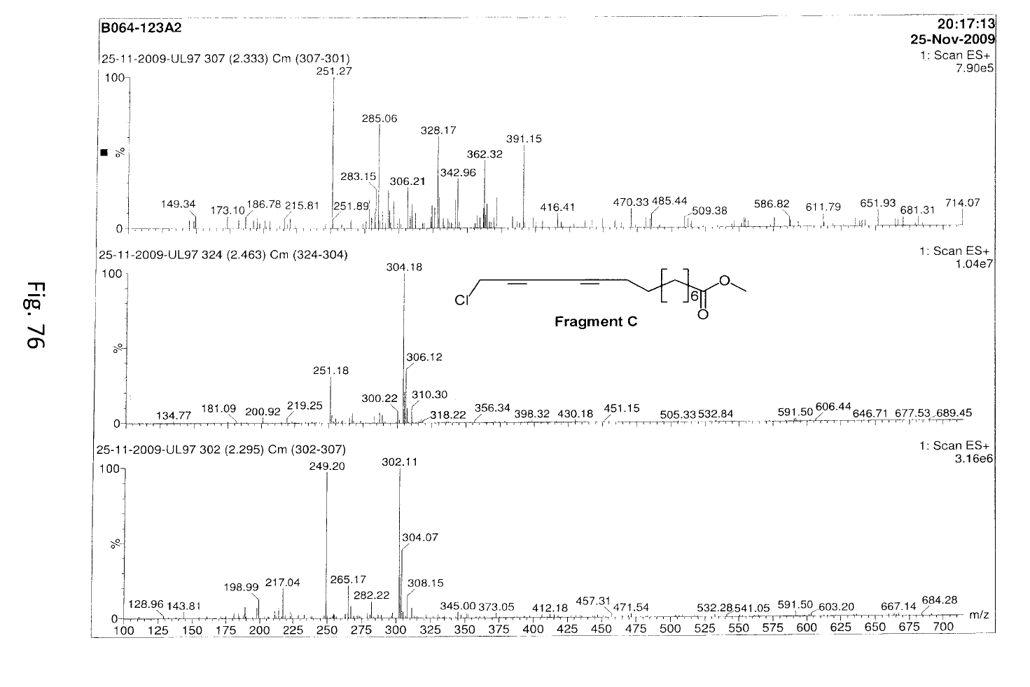

Characterization: ¹H NMR (400 MHz, CDCl₃) δ: 1.2 (m, 10H), 1.6 (m, 2H), 2 (m, 2H), 2.3 (m, 2H), 2.9 (m, 2H), 3.7 (s, 3H), 4.1 (m, 2H), 5.3-5.5 (m, 2H J=10.6), 5.7 (m, 2H J=10.8). LCMS: 78% purity, m/z=286 (m+1) (FIGS. 73-76).

### Step 14:

### LNB Reference No: B 064-055A1

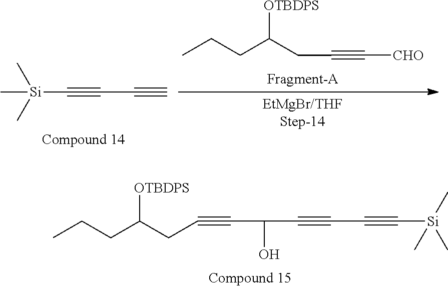

Procedure: To a stirred solution of compound 14 (2.55 g, 18.382 mmol) in dry THF (35 ml) was added EtMgBr (6.8 ml, 20.22 mmol) at 0° C. and stirred for 20 min then added Fragment A (6.9 g, 18.382 mmol) in dry THF (35 ml) stirring was continued for overnight at RT until the starting material disappeared on TLC (solvent system 20% EtOAc in pet ether, product R_{f}=0.5).

Work up: On completion of the reaction, the reaction mixture was quenched with NH₄Cl solution extracted with EtOAc (100 ml) washed with water (100 ml×2) and brine (100 ml) then solvent removed under reduced pressure to afford compound 15 (9.3 g, 98%) as brown oil (B 064-055A1).

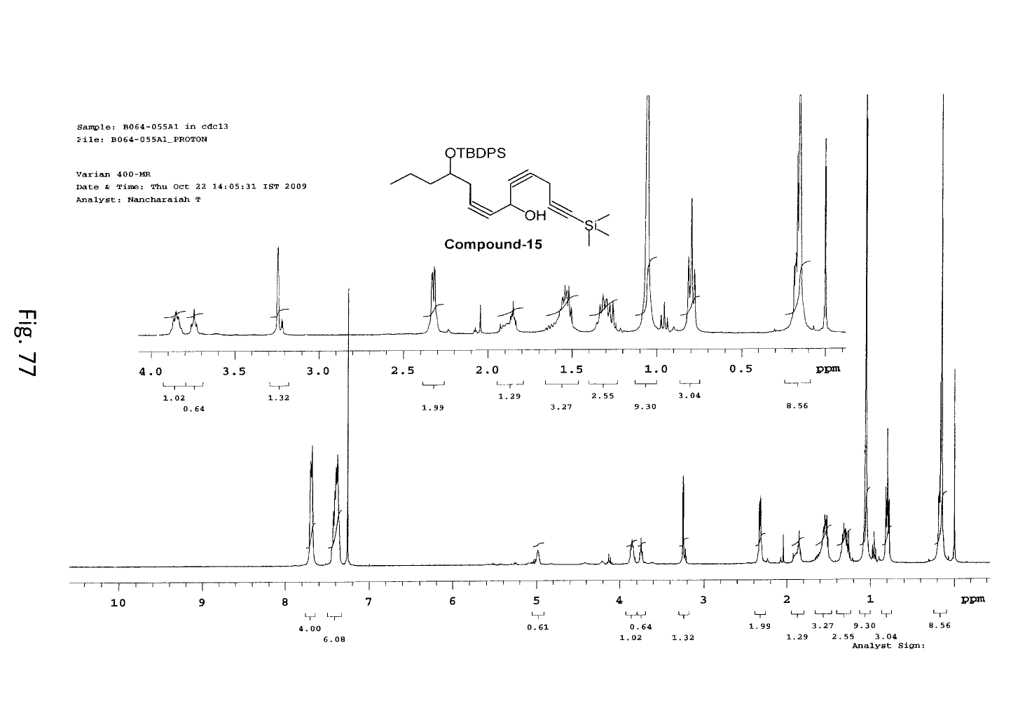

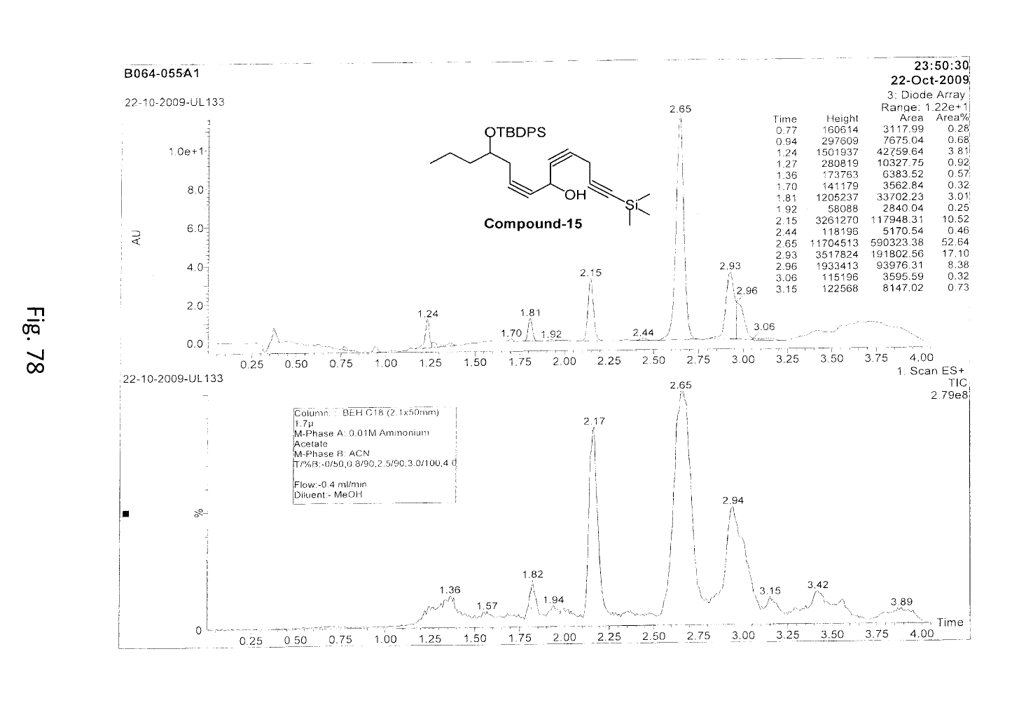

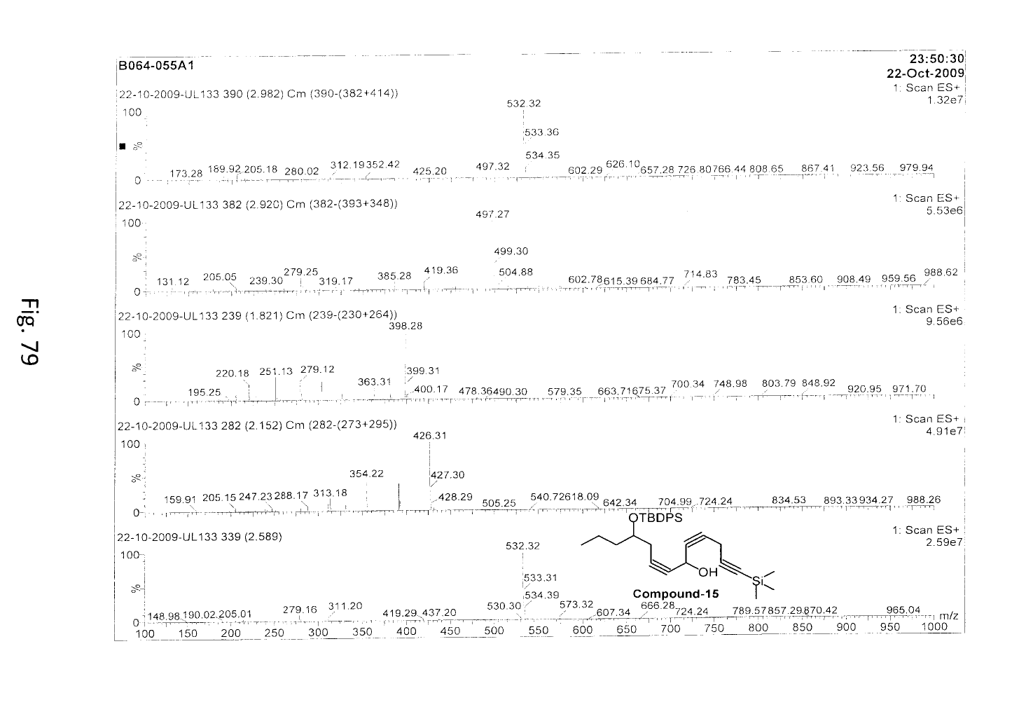

Characterization: ¹H NMR (400 MHz, CDCl₃) δ: 0.2 (s, 9H), 0.8 (t, 3H), 1.1 (s, 9H), 1.3 (m, 2H), 1.6 (m, 3H), 1.9 (m, 1H), 2.3 (m, 2H), 3.3 (m, 2H), 3.9 (m, 1H), 5 (bs, 1H), 7.4 (m, 6H), 7.7 (m, 4H). LCMS: 52.6% purity, m/z=531 (m+1) (FIGS. 77-79).

### Step 15:

### LNB Reference No: B 064-057A2

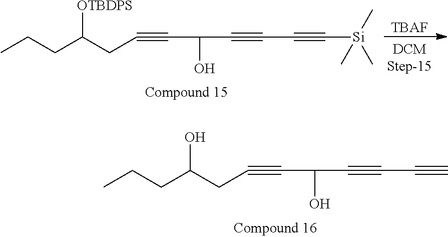

Procedure: To a stirred solution of compound 15 (4.6 g, 8.949 mmol) in dry THF (50 ml) was added TBAF (22 ml, 22.37 mmol) at 0° C. and stirred for overnight at RT until the starting material disappeared on TLC (solvent system 50% EtOAc in pet ether, product R_{f}=0.5).

Work up: On completion of the reaction, the reaction mixture was quenched with water and extracted with EtOAc (100 ml) the organic layer was washed with water (100 ml×2) brine (100 ml) then evaporated under reduced pressure to afford compound 16 (2.45 g, 64%) as brown oil (B 064-057A2).

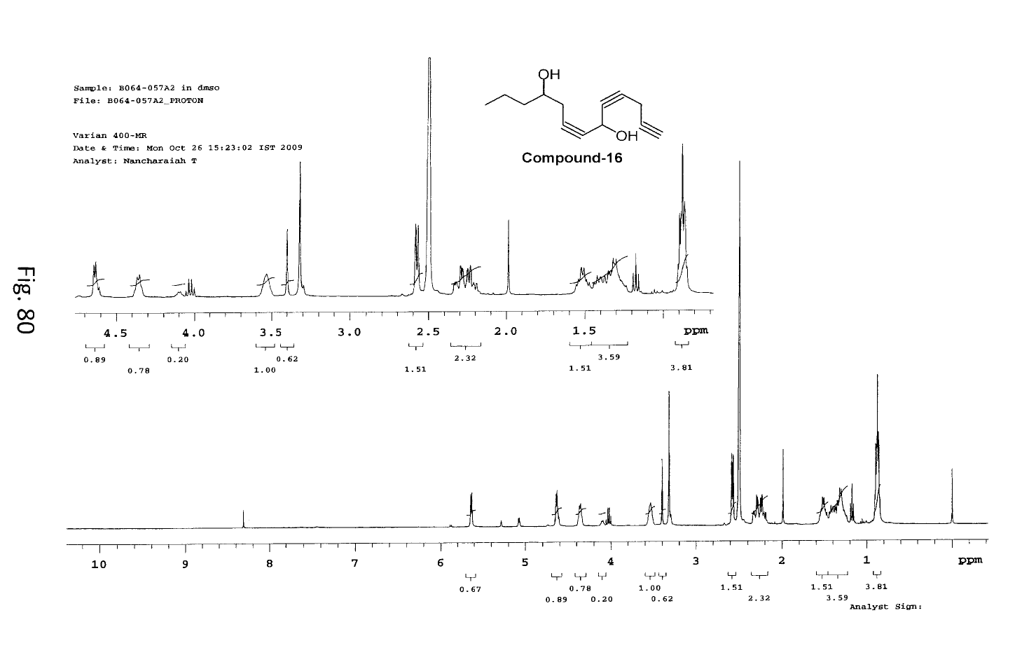

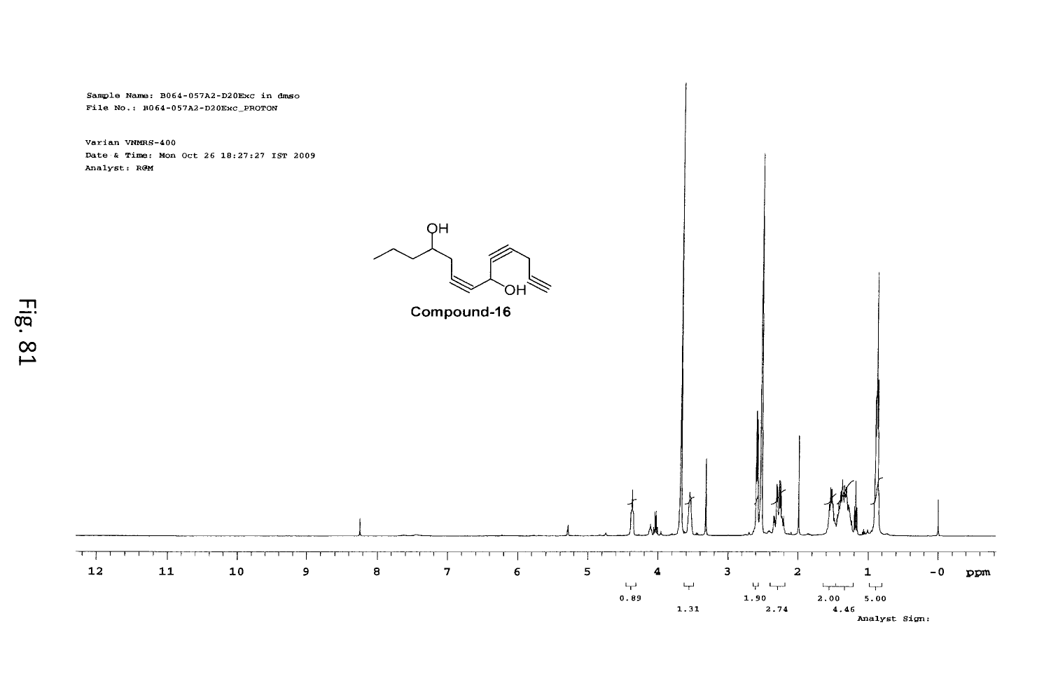

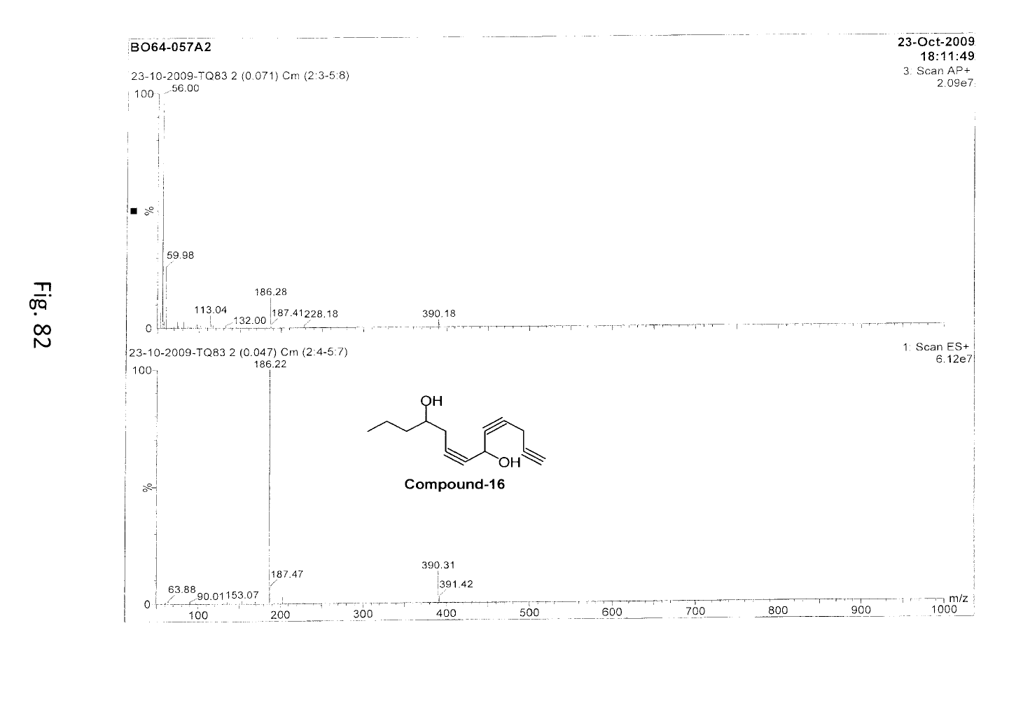

Characterization: ¹H NMR (400 MHz, DMSO) δ: 0.9 (t, 3H), 1.2-1.6 (m, 5H), 2.3 (m, 2H), 2.6 (m, 2H), 3.5 (bs, 1H), 4.4 (m, 1H), 4.6 (m, 1H), 5.6 (m, 1H) (FIGS. 80-82).

### Step 16:

### LNB Reference No: B 064-125A2

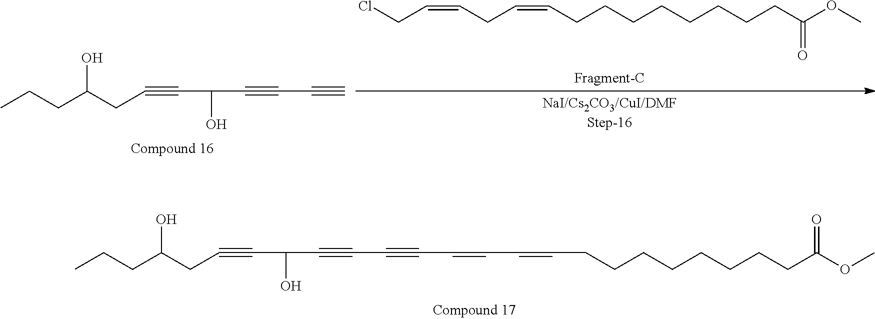

Procedure: To a stirred solution of compound 16 (0.911 g, 4.465 mmol) in dry DMF (10 ml) was added NaI (0.87 g, 5.804 mmol) Cs₂CO₃ (1.85 g, 5.804 mmol) and CuI (1.16 g, 5.804 mmol) at 0° C. and stirred for 20 min then added Fragment-C (1.27 g, 4.465 mmol) in dry DMF (10 ml) and continued stirring at RT for overnight until the starting material disappeared on TLC (solvent system 50% EtOAc in pet ether, product R_{f}=0.5).

Work up: On completion of the reaction, the reaction mixture was quenched with NH₄Cl solution and extracted with diethyl ether (50 ml×2) and washed with water (100 ml×2) and brine (100 ml) dried over anhyd Na₂SO₄ and concentrated under reduced pressure to obtain crude product which was further purified by Silica gel (100-200 mesh) column chromatography to afford compound 17 (1.22 g, 60%) as light brown oil (B 064-125A2).

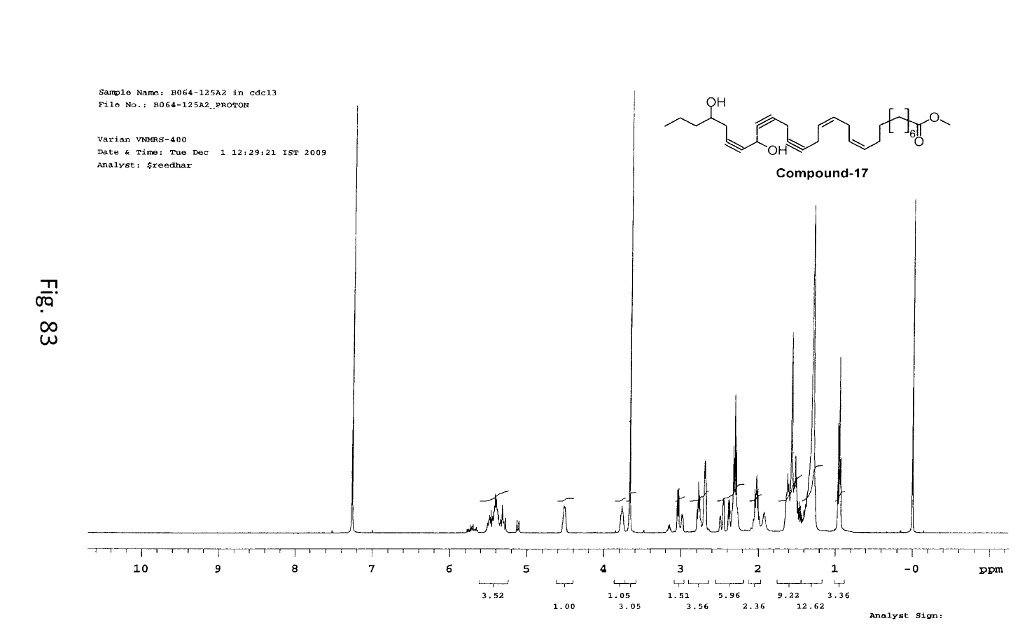

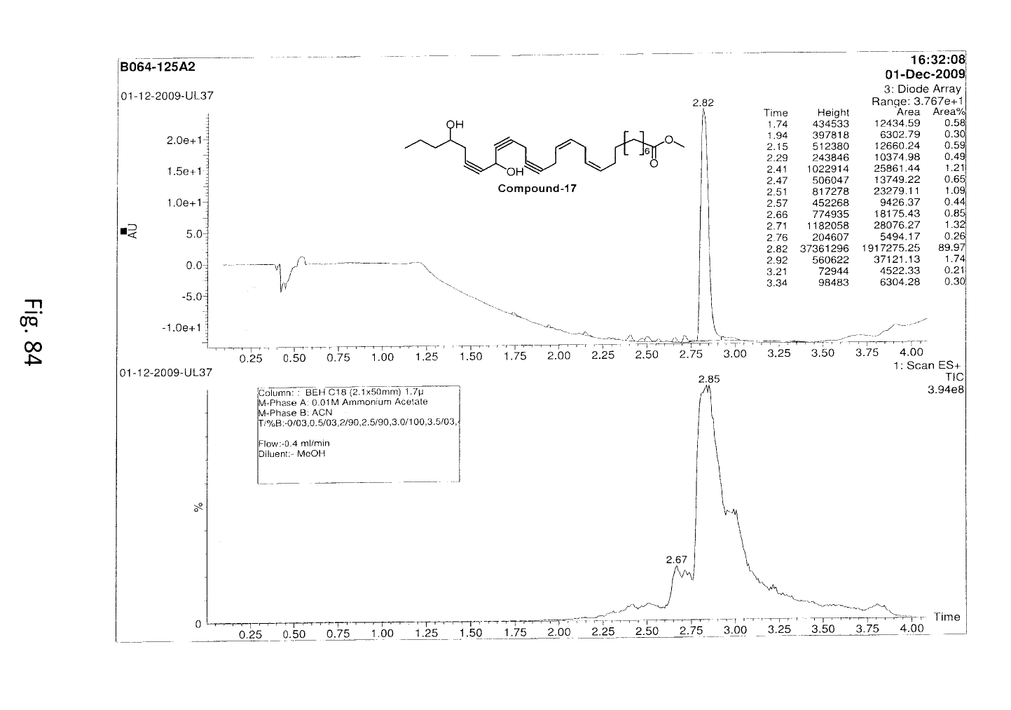

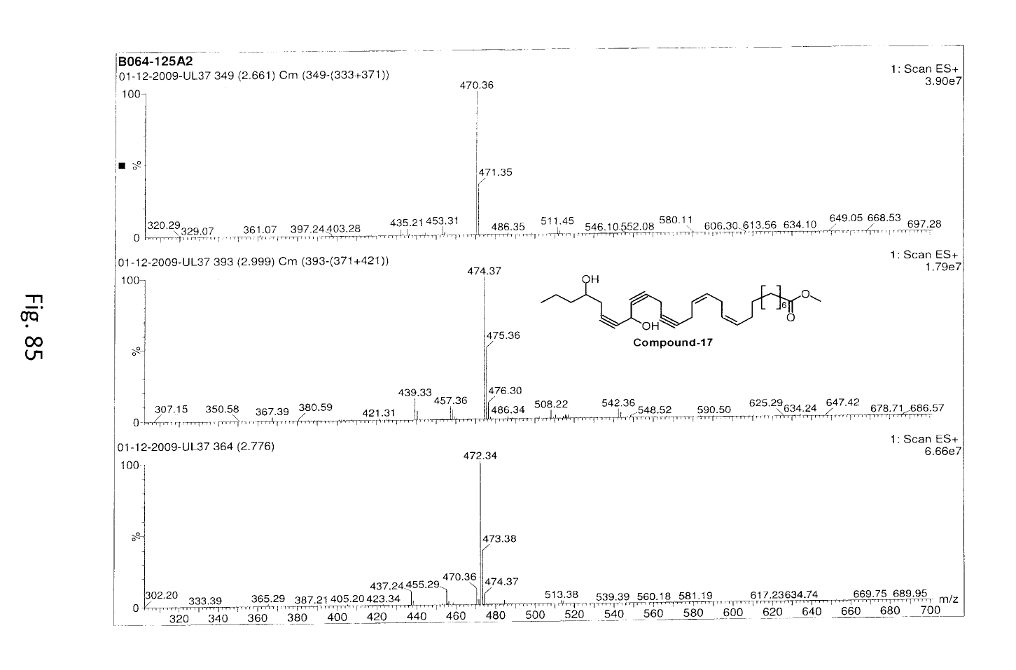

Characterization: ¹H NMR (400 MHz, CDCl₃) δ: 0.9 (t, 3H), 1.3 (m, 10H), 1.6 (m, 10H), 2.1 (m, 2H), 2.3-2.4 (m, 3H), 2.7-2.8 (m, 3H), 3.1 (m, 2H), 3.7 (s, 3H), 3.8 (bs, 1H), 4.5 (bs, 1H), 5.3-5.6 (m, 4H, J=6.4). LCMS: 90% purity, m/z=473 (m+1) (FIGS. 83-85).

### Step 17:

### LNB Reference No: B 064-131 A2-Fr-2

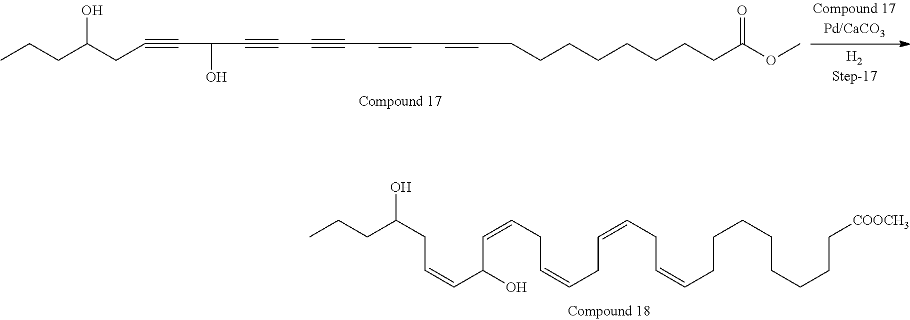

Procedure: To a stirred solution of compound 17 (730 mg) in dry methanol (18 ml) was added Pd on CaCO₃ (140 mg) and stirred under H₂ pressure for 1 h at room temperature until the starting material disappeared on TLC (solvent system 30% EtOAc in pet ether, product R_{f}=0.5).

Work up: On completion of the reaction, the reaction mix was filtered through celite bed and washed with methanol (20 ml×2) and concentrated under reduced pressure at 30° C. to afford crude compound 18 (1.2 g) which was further purified by preparative HPLC to afford pure compound 18 (120 mg) in 10% yield as colorless oil (B 064-131A2-Fr-2).

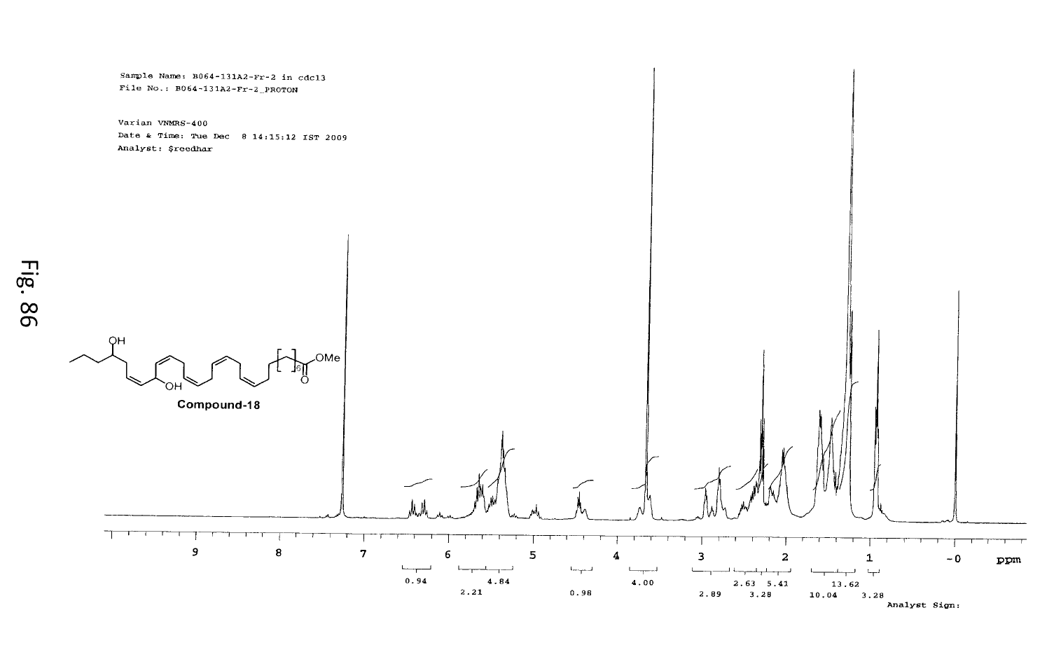

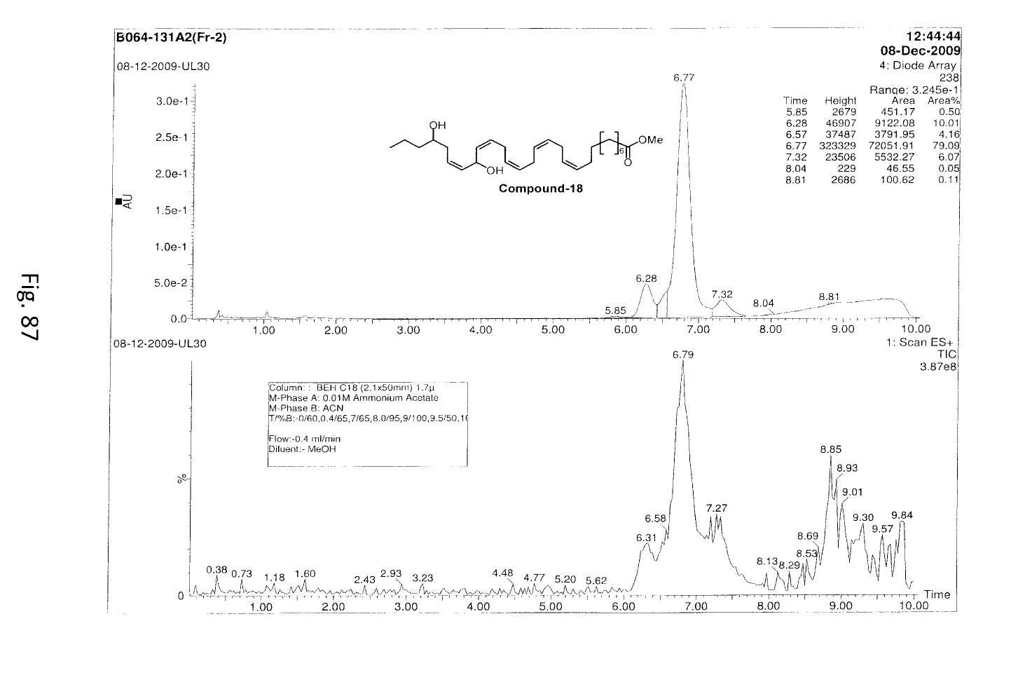

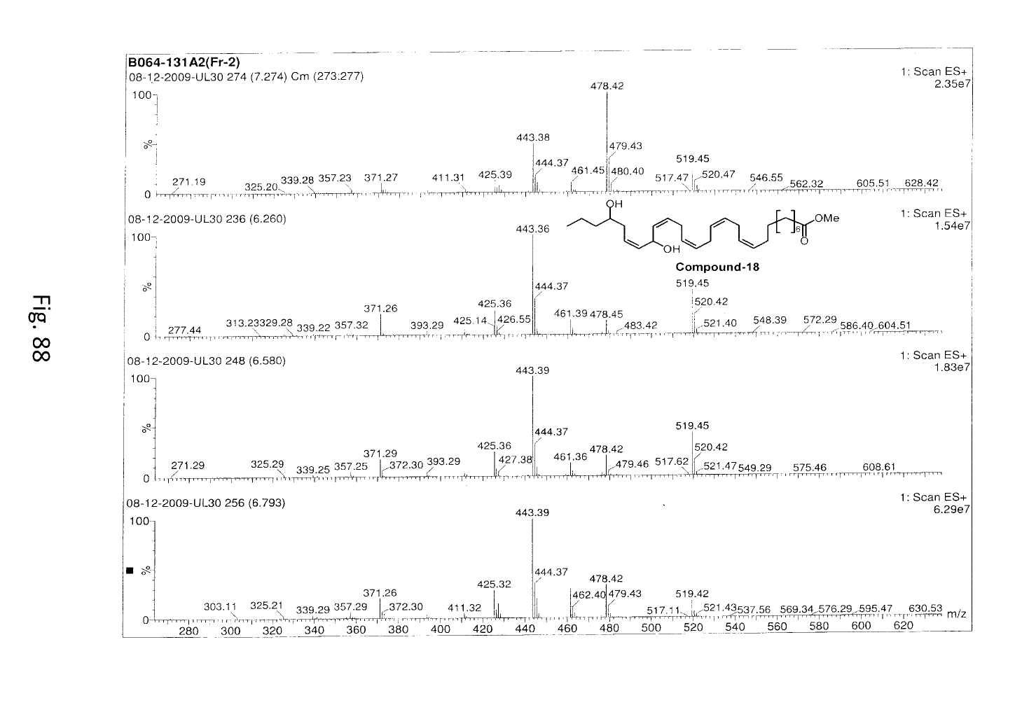

Characterization: ¹H NMR (400 MHz, CDCl₃) δ: 0.9 (t, 3H) 1.3 (m, 8H) 1.6 (m, 9H) 2.1 (m, 5H) 2.3 (m, 4H) 2.5 (m, 3H) 2.8-3.0 (m, 3H) 3.6 (s, 3H) 4.5 (m, 1H) 5.4 (m, 6H) 5.7-5.8 (m, 2H) 6.3-6.5 (m, 1H J=11). LCMS: 99% purity, m/z=461 (m+1) (FIGS. 86-88).

### Step 18:

### LNB Reference No: B 064-136A2

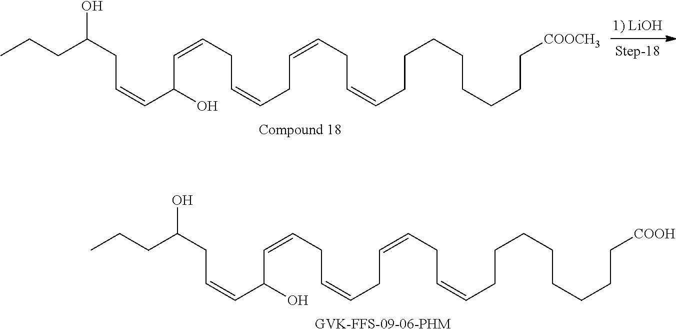

Procedure: LiOH.H₂O (55 mg, 1.3 mmol) was added to a stirred solution of compound 18 (120 mg, 0.26 mmol) in methanol (10 ml) and water (5 ml)) at 0° C. and continued stirring for overnight at RT until the starting material disappeared on TLC (solvent system 30% EtOAc in pet ether, product R_{f}=0.1).

Work up: On completion of the reaction, solvent was distilled out under reduced pressure at 30° C., then acidified with ether. HCl solution and concentrated to afford 160 mg of crude product which was further purified by preparative HPLC to afford D046-124 (11 mg, 9%) as a colorless oil (B 064-136A2).

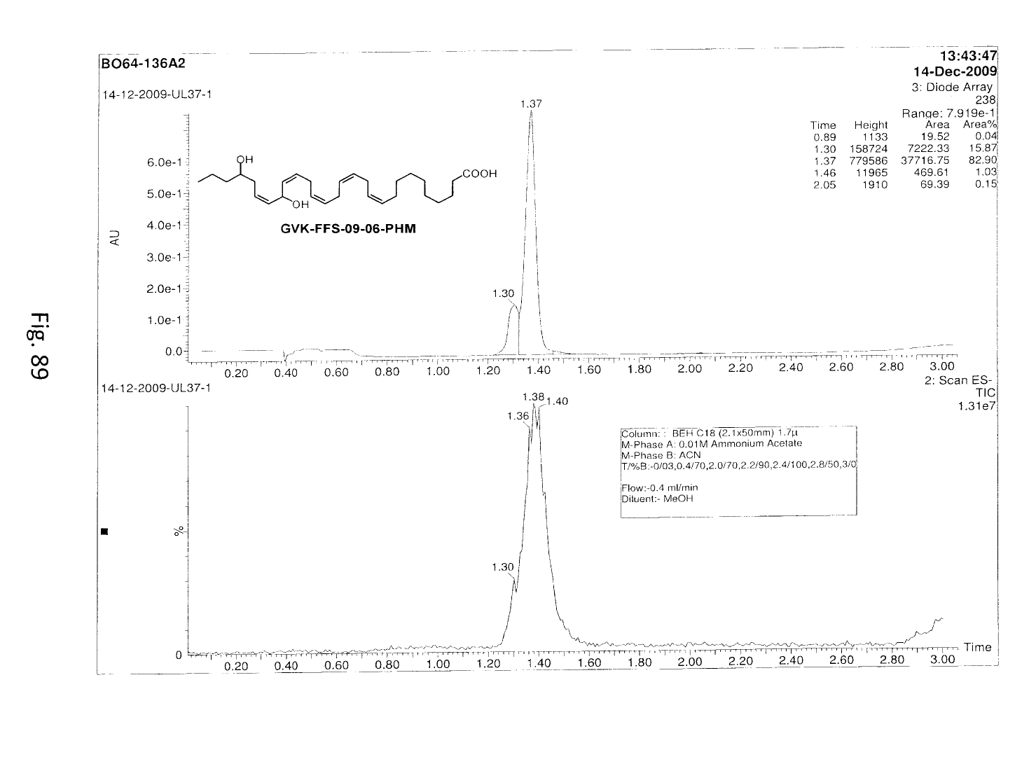

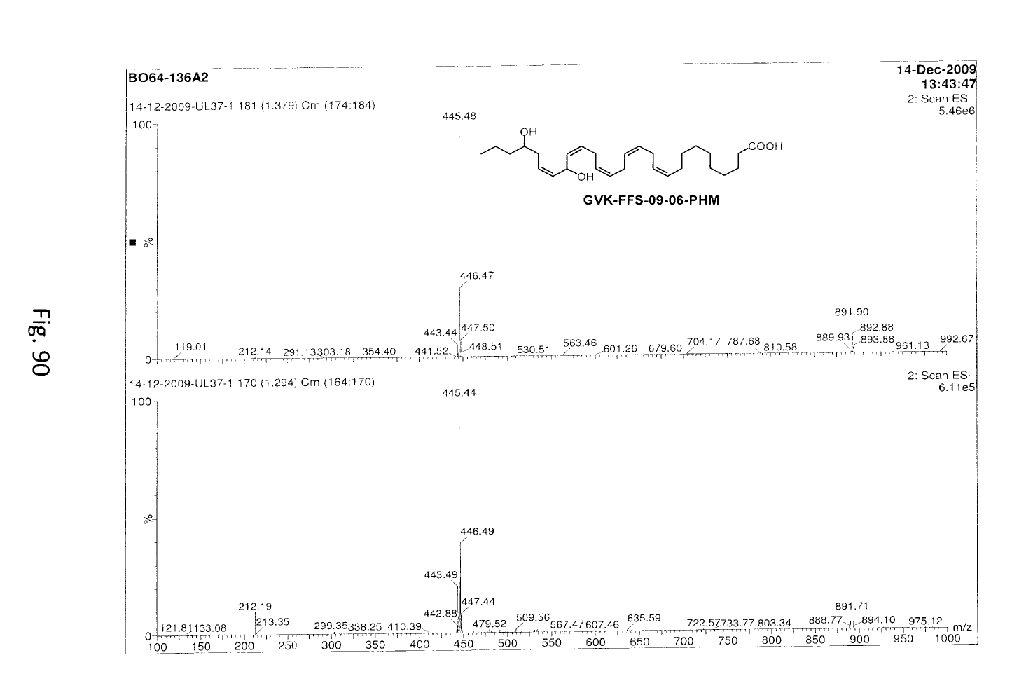

Characterization: LCMS: 98.7% purity, m/z=445 (m-1) (FIGS. 89-90).

One or more currently preferred embodiments have been described by way of example. It will be apparent to persons skilled in the art that a number of variations and modifications can be made without departing from the scope of the invention as defined in the claims.

### REFERENCES

1. Roy H K, Backman V, Goldberg M J. Colon cancer screening: the good, the bad, and the ugly. Arch Intern Med 2006; 166:2177-9.
2. Ouyang D L, Chen J J, Getzenberg R H, Schoen R E. Noninvasive testing for colorectal cancer: a review. Am J Gastroenterol 2005; 100:1393-403.
3. Davies R J, Miller R, Coleman N. Colorectal cancer screening: prospects for molecular stool analysis. Nat Rev Cancer 2005; 5:199-209.
4. Kleivi K, Lind G E, Diep C B, Meling G I, Brandal L T, Nesland J M, Myklebost O, Rognum T O, Giercksky K E, Skotheim R I, Lothe R A. Gene expression profiles of primary colorectal carcinomas, liver metastases, and carcinomatoses. Mol Cancer 2007; 6:2.
5. Solmi R, Ugolini G, Rosati G, Zanotti S, Lauriola M, Montroni I, del Governatore M, Caira A, Taffurelli M, Santini D, Coppola D, Guidotti L, Carinci P, Strippoli P. Microarray-based identification and RT-PCR test screening for epithelial specific mRNAs in peripheral blood of patients with colon cancer. BMC Cancer 2006; 6:250.
6. Komori T, Takemasa I, Higuchi H, Yamasaki M, Ikeda M, Yamamoto H, Ohue M, Nakamori S, Sekimoto M, Matsubara K, Monden M. Identification of differentially expressed genes involved in colorectal carcinogenesis using a cDNA microarray. J Exp Clin Cancer Res 2004; 23:521-7.
7. Hegde P, Qi R, Gaspard R, Abernathy K, Dharap S, Earle-Hughes J, Gay C, Nwokekeh N U, Chen T, Saeed A I, Sharov V, Lee N H, Yeatman T J, Quackenbush J. Identification of tumor markers in models of human colorectal cancer using a 19,200-element complementary DNA microarray. Cancer Res 2001; 61:7792-7.
8. Kitahara O, Furukawa Y, Tanaka T, Kihara C, Ono K, Yanagawa R, Nita M E, Takagi T, Nakamura Y, Tsunoda T. Alterations of gene expression during colorectal carcinogenesis revealed by cDNA microarrays after laser-capture microdissection of tumor tissues and normal epithelia. Cancer Res 2001; 61:3544-9.
9. Notterman D A, Alon U, Sierk A J, Levine A J. Transcriptional gene expression profiles of colorectal adenoma, adenocarcinoma, and normal tissue examined by oligonucleotide arrays. Cancer Res 2001; 61:3124-30.
10. Takemasa I, Higuchi H, Yamamoto H, Sekimoto M, Tomita N, Nakamori S, Matoba R, Monden M, Matsubara K. Construction of preferential cDNA microarray specialized for human colorectal carcinoma: molecular sketch of colorectal cancer. Biochem Biophys Res Commun 2001; 285:1244-9.
11. Backert S, Gelos M, Kobalz U, Hanski M L, Bohm C, Mann B, Lovin N, Gratchev A, Mansmann U, Moyer M P, Riecken E O, Hanski C. Differential gene expression in colon carcinoma cells and tissues detected with a cDNA array. Int J Cancer 1999; 82:868-74.
12. Mori Y, Cai K, Cheng Y, Wang S, Paun B, Hamilton J P, Jin Z, Sato F, Berki A T, Kan T, Ito T, Mantzur C, Abraham J M, Meltzer S J. A genome-wide search identifies epigenetic silencing of somatostatin, tachykinin-1, and 5 other genes in colon cancer. Gastroenterology 2006; 131:797-808.
13. Chen W D, Han Z J, Skoletsky J, Olson J, Sah J, Myeroff L, Platzer P, Lu S, Dawson D, Willis J, Pretlow T P, Lutterbaugh J, Kasturi L, Willson J K, Rao J S, Shuber A, Markowitz S D. Detection in fecal DNA of colon cancer-specific methylation of the nonexpressed vimentin gene. J Natl Cancer Inst 2005; 97:1124-32.
14. Leung W K, To K F, Man E P, Chan M W, Bai A H, Hui A J, Chan F K, Sung J J. Quantitativedetection of promoter hypermethylation in multiple genes in the serum of patients with colorectal cancer. Am J Gastroenterol 2005; 100:2274-9.
15. Ward D G, Suggett N, Cheng Y, Wei W, Johnson H, Billingham L J, Ismail T, Wakelam M J, Johnson P J, Martin A. Identification of serum biomarkers for colon cancer by proteomic analysis. Br J Cancer 2006; 94:1898-905.
16. Lou J, Fatima N, Xiao Z, Stauffer S, Smythers G, Greenwald P, Ali I U. Proteomic profiling identifies cyclooxygenase-2-independent global proteomic changes by celecoxib in colorectal cancer cells. Cancer Epidemiol Biomarkers Prey 2006; 15:1598-606.
17. Mazzanti R, Solazzo M, Fantappie O, Elfering S, Pantaleo P, Bechi P, Cianchi F, Ettl A, Giulivi C. Differential expression proteomics of human colon cancer. Am J Physiol Gastrointest Liver Physiol 2006; 290:G1329-38.
18. Roblick U J, Hirschberg D, Habermann J K, Palmberg C, Becker S, Kruger S, Gustafsson M, Bruch H P, Franzen B, Ried T, Bergmann T, Auer G, Jornvall H. Sequential proteome alterations during genesis and progression of colon cancer. Cell Mol Life Sci 2004; 61:1246-55.
19. de la Chapelle A. Genetic predisposition to colorectal cancer. Nat Rev Cancer 2004; 4:769-80.
20. Marshall J R. Prevention of colorectal cancer: diet, chemoprevention, and lifestyle. Gastroenterol Clin North Am 2008; 37:73-82, vi.
21. Fearnhead N S, Wilding J L, Bodmer W F. Genetics of colorectal cancer: hereditary aspects and overview of colorectal tumorigenesis. Br Med Bull 2002; 64:27-43.
22. McGarr S E, Ridlon J M, Hylemon P B. Diet, anaerobic bacterial metabolism, and colon cancer: a review of the literature. J Clin Gastroenterol 2005; 39:98-109.
23. Aharoni A, Ric de Vos C H, Verhoeven H A, Maliepaard C A, Kruppa G, Bino R, Goodenowe D B. Nontargeted metabolome analysis by use of Fourier Transform Ion Cyclotron Mass Spectrometry. Omics 2002; 6:217-34.
24. Dettmer K, Aronov P A, Hammock B D. Mass spectrometry-based metabolomics. Mass Spectrom Rev 2007; 26:51-78.
25. Want E J, Nordstrom A, Morita H, Siuzdak G. From exogenous to endogenous: the inevitable imprint of mass spectrometry in metabolomics. J Proteome Res 2007; 6:459-68.
26. Pinto D M, Boyd R K, Volmer D A. Ultra-high resolution for mass spectrometric analysis of complex and low-abundance mixtures—the emergence of FTICR-MS as an essential analytical tool. Anal Bioanal Chem 2002; 373:378-89.
27. Breitling R, Ritchie S, Goodenowe D, Stewart M L, Barrett M P. Ab initio prediction of metabolic networks using Fourier transform mass spectrometry data. Metabolomics 2006; 2:155-164.
28. Zytkovicz T H, Fitzgerald E F, Marsden D, Larson C A, Shih V E, Johnson D M, Strauss A W, Comeau A M, Eaton R B, Grady G F. Tandem mass spectrometric analysis for amino, organic, and fatty acid disorders in newborn dried blood spots: a two-year summary from the New England Newborn Screening Program. Clin Chem 2001; 47:1945-55.
29. Hong S, Gronert K, Devchand P R, Moussignac R L, Serhan C N. Novel docosatrienes and 17S -resolvins generated from docosahexaenoic acid in murine brain, human blood, and glial cells. Autacoids in anti-inflammation. J Biol Chem 2003; 278:14677-87.
30. Hong S, Lu Y, Yang R, Gotlinger K H, Petasis N A, Serhan C N. Resolvin D1, protectin D1, and related docosahexaenoic acid-derived products: Analysis via electrospray/low energy tandem mass spectrometry based on spectra and fragmentation mechanisms. J Am Soc Mass Spectrom 2007; 18:128-44.
31. Serhan C N, Hong S, Gronert K, Colgan S P, Devchand P R, Mirick G, Moussignac R L. Resolvins: a family of bioactive products of omega-3 fatty acid transformation circuits initiated by aspirin treatment that counter proinflammation signals. J Exp Med 2002; 196:1025-37.
32. Lu Y, Hong S, Yang R, Uddin J, Gotlinger K H, Petasis N A, Serhan C N. Identification of endogenous resolvin E1 and other lipid mediators derived from eicosapentaenoic acid via electrospray low-energy tandem mass spectrometry: spectra and fragmentation mechanisms. Rapid Commun Mass Spectrom 2007; 21:7-22.
33. Murphy R C, Fiedler J, Hevko J. Analysis of nonvolatile lipids by mass spectrometry. Chem Rev 2001; 101:479-526.
34. Poulos A, Beckman K, Johnson D W, Paton B C, Robinson B S, Sharp P, Usher S, Singh H. Very long-chain fatty acids in peroxisomal disease. Adv Exp Med Biol 1992; 318:331-40.
35. Johnson D W, Trinh M U. Analysis of isomeric long-chain hydroxy fatty acids by tandem mass spectrometry: application to the diagnosis of long-chain 3-hydroxyacyl CoA dehydrogenase deficiency. Rapid Commun Mass Spectrom 2003; 17:171-5.
36. Lim J Y, Cho J Y, Paik Y H, Chang Y S, Kim H G. Diagnostic application of serum proteomic patterns in gastric cancer patients by ProteinChip surface-enhanced laser desorption/ionization time-of-flight mass spectrometry. Int J Biol Markers 2007; 22:281-6.
37. Su Y, Shen J, Qian H, Ma H, Ji J, Ma L, Zhang W, Meng L, Li Z, Wu J, Jin G, Zhang J, Shou C. Diagnosis of gastric cancer using decision tree classification of mass spectral data. Cancer Sci 2007; 98:37-43.
38. Chen Y D, Zheng S, Yu J K, Hu X. Artificial neural networks analysis of surface-enhanced laser desorption/ionization mass spectra of serum protein pattern distinguishes colorectal cancer from healthy population. Clin Cancer Res 2004; 10:8380-5.
39. Ringner M, Peterson C, Khan J. Analyzing array data using supervised methods. Pharmacogenomics 2002; 3:403-15.
40. Baggerly K A, Morris J S, Coombes K R. Reproducibility of SELDI-TOF protein patterns in serum: comparing datasets from different experiments. Bioinformatics 2004; 20:777-85.
41. L. V. H. Meta-Analysis. Journal of Educational Statistics 1992; 17:279-296.
42. Fisher R A. Statistical methods for research workers Oliver & Boyd, 1932.
43. Marinangeli C P, Kassis A N, Jain D, Ebine N, Cunnane S C, Jones P J. Comparison of composition and absorption of sugarcane policosanols. Br J Nutr 2007; 97:381-8.
44. Wang M F, Lian H Z, Mao L, Zhou J P, Gong H J, Qian B Y, Fang Y, Li J. Comparison of various extraction methods for policosanol from rice bran wax and establishment of chromatographic fingerprint of policosanol. J Agric Food Chem 2007; 55:5552-8.
45. Collins J F, Lieberman D A, Durbin T E, Weiss D G. Accuracy of screening for fecal occult blood on a single stool sample obtained by digital rectal examination: a comparison with recommended sampling practice. Ann Intern Med 2005; 142:81-5.
46. Das U N. Essential fatty acids: biochemistry, physiology and pathology. Biotechnol J, 2006; 1:420-39.
47. Das U N. Folic acid and polyunsaturated fatty acids improve cognitive function and prevent depression, dementia, and Alzheimer's disease—but how and why? Prostaglandins Leukot Essent Fatty Acids 2008; 78:11-9.
48. Arita M, Yoshida M, Hong S, Tjonahen E, Glickman J N, Petasis N A, Blumberg R S, Serhan C N. Resolvin E1, an endogenous lipid mediator derived from omega-3 eicosapentaenoic acid, protects against 2,4,6-trinitrobenzene sulfonic acid-induced colitis. Proc Natl Acad Sci USA 2005; 102:7671-6.
49. Goh J, Baird A W, O'Keane C, Watson R W, Cottell D, Bernasconi G, Petasis N A, Godson C, Brady H R, MacMathuna P. Lipoxin A(4) and aspirin-triggered 15-epi-lipoxin A(4) antagonize TNF-alpha-stimulated neutrophil-enterocyte interactions in vitro and attenuate TNF-alpha-induced chemokine release and colonocyte apoptosis in human intestinal mucosa ex vivo J Immunol 2001; 167:2772-80.
50. Gewirtz A T, Collier-Hyams L S, Young A N, Kucharzik T, Guilford W J, Parkinson J F, Williams I R, Neish A S, Madara J L. Lipoxin a4 analogs attenuate induction of intestinal epithelial proinflammatory gene expression and reduce the severity of dextran sodium sulfate-induced colitis. J Immunol 2002; 168:5260-7.
51. Serhan C N. Controlling the resolution of acute inflammation: a new genus of dual anti-inflammatory and proresolving mediators. J Periodontol 2008; 79:1520-6.
52. Schwab J M, Chiang N, Arita M, Serhan C N. Resolvin E1 and protectin D1 activate inflammation-resolution programmes. Nature 2007; 447:869-74.
53. Serhan C N, Gotlinger K, Hong S, Lu Y, Siegelman J, Baer T, Yang R, Colgan S P, Petasis N A. Anti-inflammatory actions of neuroprotectin D1/protectin D1 and its natural stereoisomers: assignments of dihydroxy-containing docosatrienes. J Immunol 2006; 176:1848-59.
54. Serhan C N. Novel chemical mediators in the resolution of inflammation: resolvins and protectins. Anesthesiol Clin 2006; 24:341-64.
55. Schwab J M, Serhan C N. Lipoxins and new lipid mediators in the resolution of inflammation. Curr Opin Pharmacol 2006; 6:414-20.

1. A compound of formula (I): 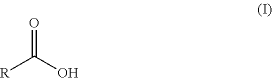
wherein R represents a hydroxy substituted C₂₄-C₄₀ straight chain aliphatic group containing at least one double bond in the carbon chain; and at least one carbon in the chain is substituted with a hydroxy group.

2. The compound of claim 1, wherein R is a C₂₈-C₃₆ aliphatic group.

3. The compound of claim 2, wherein 2, 3 or 4 carbons in the chain are substituted with a hydroxy group.

4. The compound of claim 1, selected from the following group of structures:

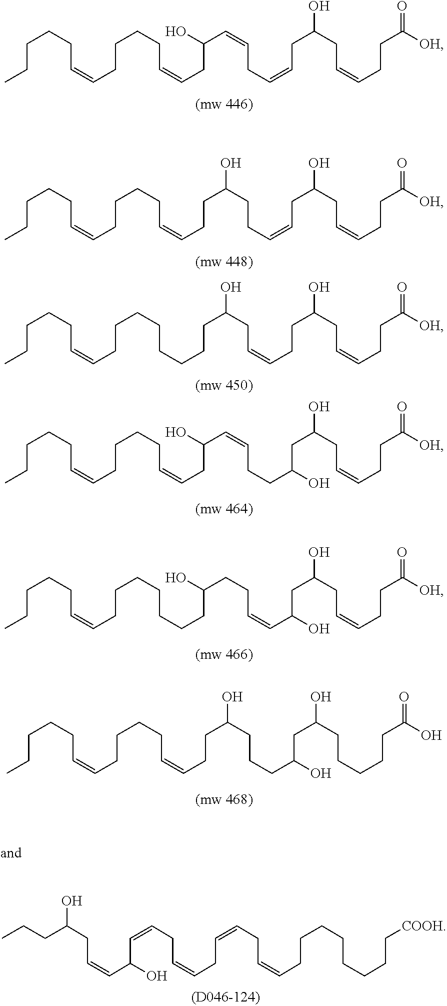

5. The compound of claim 1, which is compound D046-124 of the following structure:

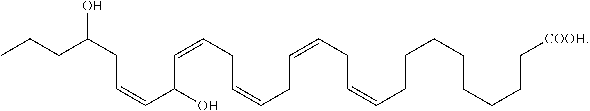

6. A method of treating or preventing colorectal cancer (CRC) in a subject, comprising administering to the subject in an amount sufficient to treat or prevent CRC a compound of formula (I) as defined in claim 1.

7-8. (canceled)

9. The method of claim 6, wherein the compound is selected from the following group of structures:

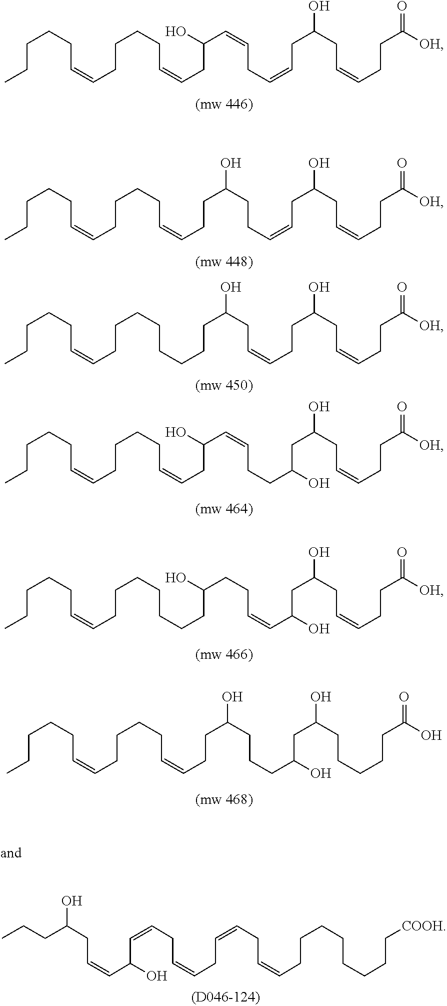

10. (canceled)

11. A method of inhibiting tumor growth in a subject, comprising administering to the subject in an amount sufficient to inhibit growth of the tumor a compound of formula (I) as defined in claim 1.

12-13. (canceled)

14. The method of claim 11, wherein the compound is selected from the following group of structures:

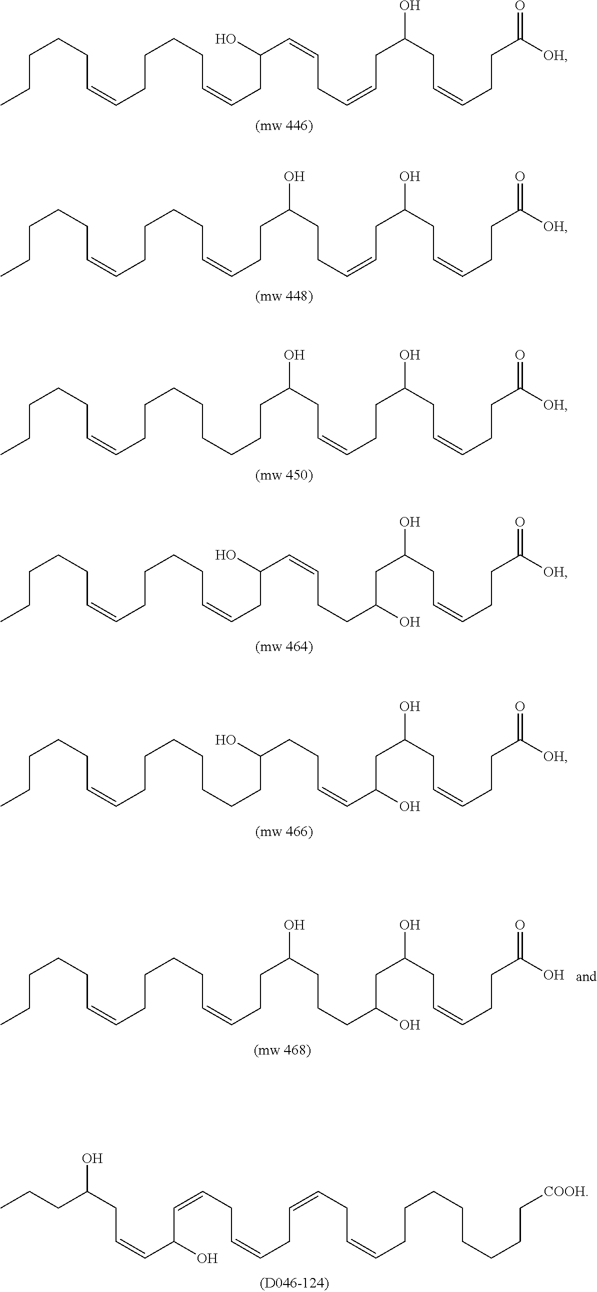

15. (canceled)

16. A method of treating or preventing a gastrointestinal (GI) disorder in a subject, comprising administering to the subject in an amount sufficient to treat, prevent or mitigate the GI disorder in the subject a compound of formula (I) as defined in claim 1.

17-18. (canceled)

19. The method of claim 16, wherein the compound is selected from the following group of structures:

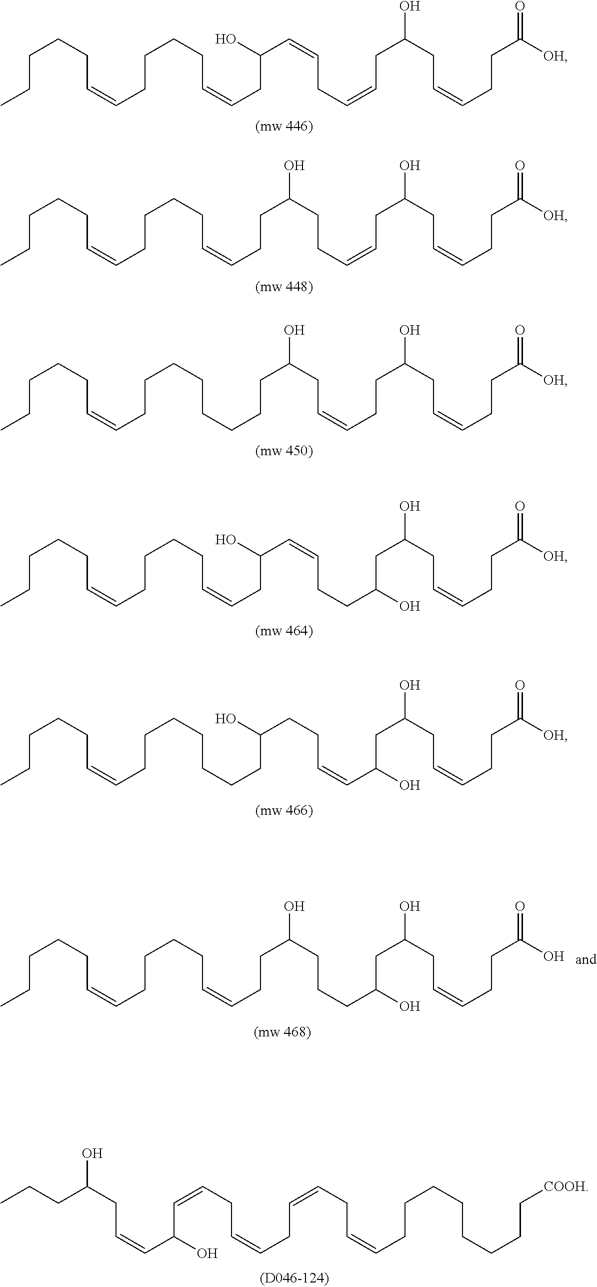

20. (canceled)

21. A method of treating or preventing inflammation and/or an inflammation-related disorder in a subject in need thereof, comprising administering to the subject in an amount effective to prevent said inflammation and/or inflammation-related disorder a compound of formula (I) as defined in claim 1.

22-23. (canceled)

24. The method of claim 21, wherein the compound is selected from the following group of structures:

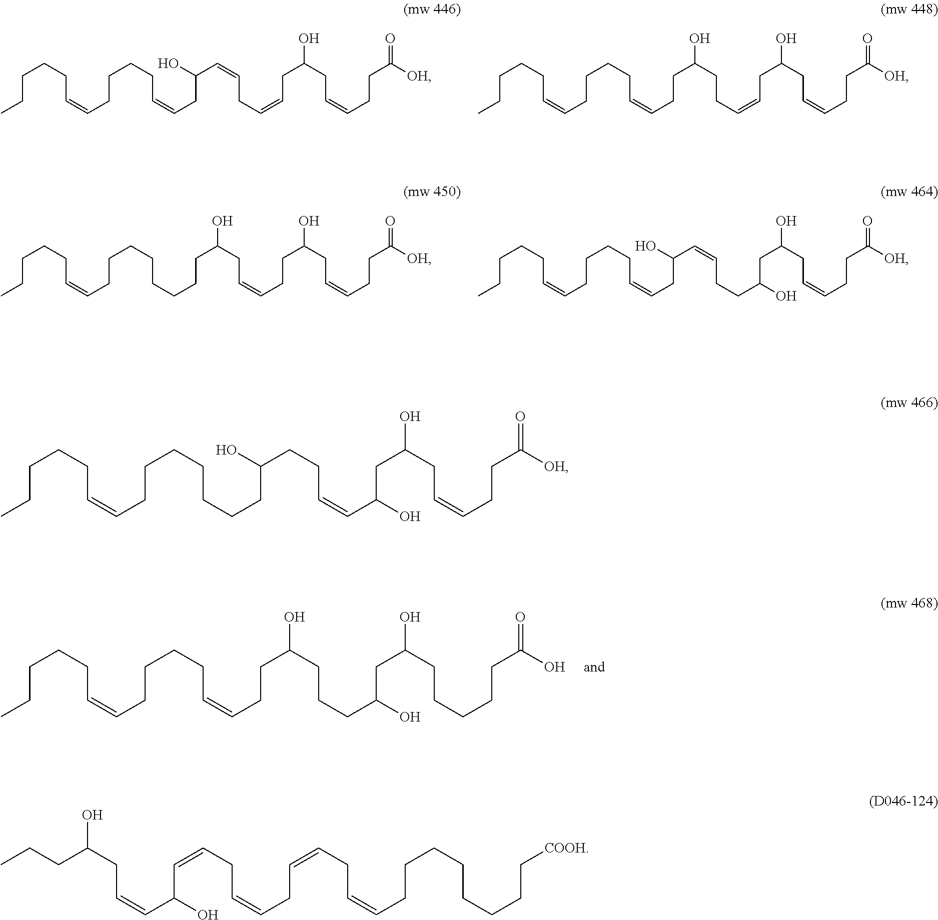

25. (canceled)

26. A method of treating or preventing a hydroxylated polyunsaturated ultra long-chain fatty acid (hPULCFA) deficiency disorder (hPDD) in a subject, comprising administering in an amount sufficient to treat or prevent hPDD in the subject a compound of formula (I) as defined in claim 1.

27-28. (canceled)

29. The method of claim 26, wherein the compound is selected from the following group of structures:

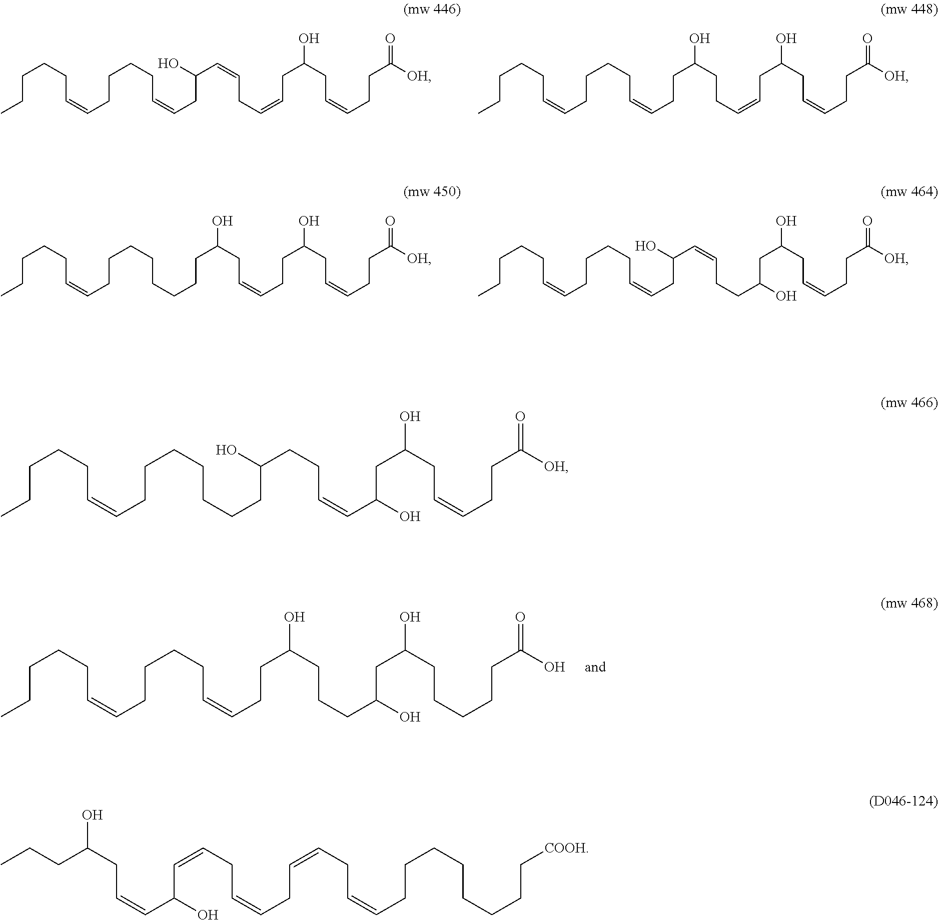

30. (canceled)

31. The method of claim 26, wherein the amount of compound administered is effective to elevate or restore hPULCFA levels.

32. A method for diagnosing a subject's CRC health state or change in health state, or for diagnosing CRC or the risk of CRC in a subject, comprising steps of:
a) analyzing a sample from the subject to quantify in said sample the amount of a compound of formula (I) as defined in claim 1;
b) comparing the quantified amount of the compound in the subject sample to a corresponding amount of the compound in one or more than one reference sample to determine the presence or absence of an increase or decrease in the amount of the compound in the subject sample; and
c) using said increase or decrease for diagnosing the subject's CRC health state or change in health state, or for diagnosing CRC or the risk of CRC in the subject.

33-34. (canceled)

35. The method of claim 32, wherein the compound is selected from the following group of structures:

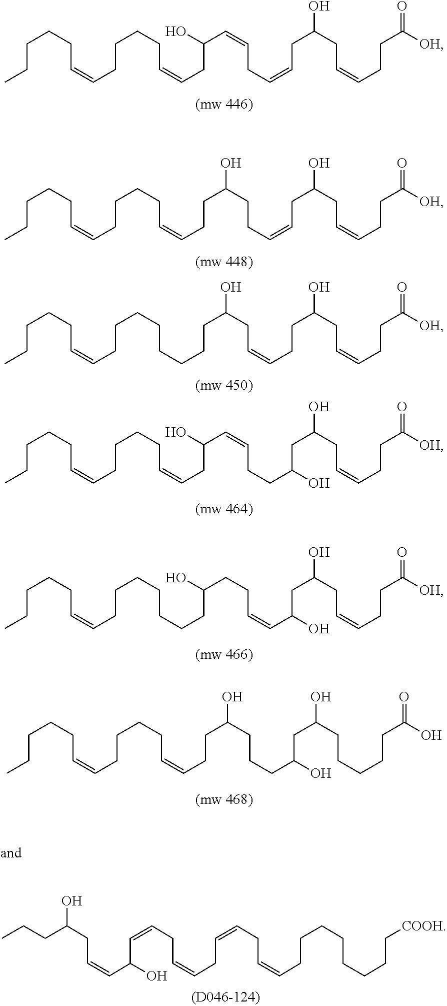

36. The method of claim 32, wherein the sample is a blood sample from said subject and is analyzed in step a) by mass spectrometry to obtain accurate mass intensity data for said compound, and the accurate mass intensity data is compared in step b) to corresponding accurate mass intensity data obtained from the one or more than one reference sample to identify an increase or decrease in accurate mass intensity.

37. The method of claim 32, wherein the sample is a blood sample from said subject and is analyzed in step a) by tandem mass spectrometry, NMR or ELISA.

38. A method of diagnosing a hPULCFA Deficiency Disorder (hPDD) in a subject, comprising:
a) analyzing a sample from the subject to quantify in the sample the amount of a compound of formula (I) as defined in claim 1;
b) comparing the quantified amount of the compound in the subject sample to a corresponding amount of the compound in one or more than one reference sample to determine the presence or absence of an increase or decrease in the amount of the compound in the subject sample; and
c) using the increase or decrease for diagnosing hPDD in the subject.

39-40. (canceled)

41. The method of claim 38, wherein the compound is selected from the following group of structures:

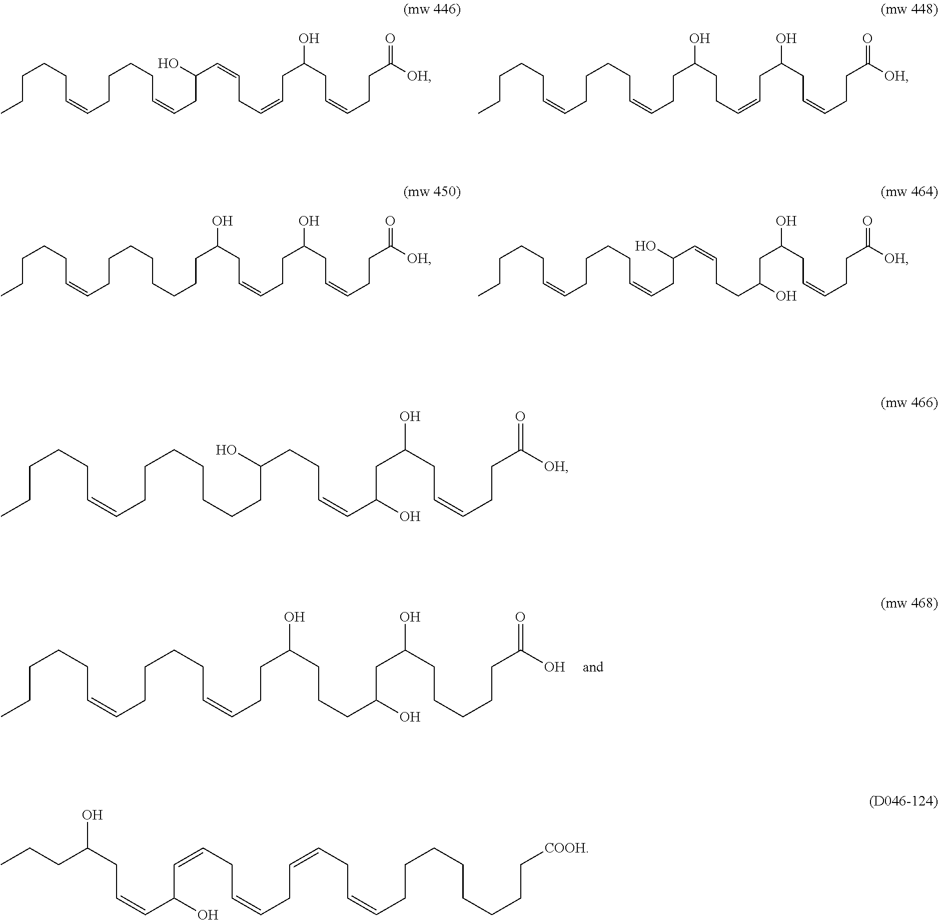

42. The method of claim 38, wherein the sample is a blood sample from said subject and is analyzed in step a) by mass spectrometry to obtain accurate mass intensity data for said compound, and the accurate mass intensity data is compared in step b) to corresponding accurate mass intensity data obtained from the one or more than one reference sample to identify an increase or decrease in accurate mass intensity.

43. The method of claim 38, wherein the sample is a blood sample from said subject and is analyzed in step a) by tandem mass spectrometry, NMR or ELISA.

44. A method of diagnosing inflammation or an inflammatory disease comprising:
a) analyzing a sample from the subject to quantify in the sample the amount of a compound of formula (I) as defined in claim 1;
b) comparing the quantified amount of the compound in the subject sample to a corresponding amount of the compound in one or more than one reference sample to determine the presence or absence of an increase or decrease in the amount of the compound in the subject sample; and
c) using the increase or decrease for diagnosing inflammation or an inflammatory disease in the subject.

45-46. (canceled)

47. The method of claim 44, wherein the compound is selected from the following group of structures:

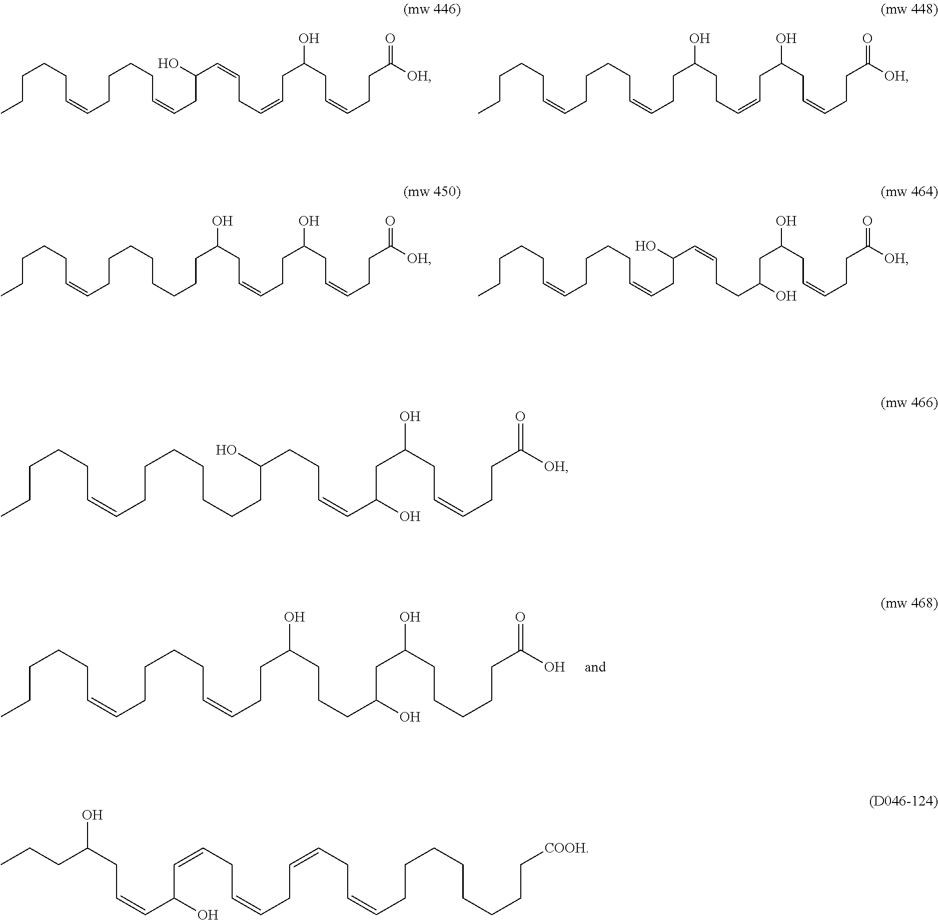

48. The method of claim 44, wherein the sample is a blood sample from said subject and is analyzed in step a) by mass spectrometry to obtain accurate mass intensity data for said compound, and the accurate mass intensity data is compared in step b) to corresponding accurate mass intensity data obtained from the one or more than one reference sample to identify an increase or decrease in accurate mass intensity.

49. The method of claim 44, wherein the sample is a blood sample from said subject and is analyzed in step a) by tandem mass spectrometry, NMR or ELISA.

50. The method of claim 44, wherein the inflammation is caused by, or the inflammatory disease includes, a GI disorder selected from IBD, Crohn's, and/or colitis.

51. A method of monitoring the effect of an anti-inflammatory drug comprising:
a) analyzing a sample from a subject treated with said anti-inflammatory drug to quantify in the sample the amount of a compound of formula (I) as defined in claim 1;
and
b) comparing the quantified amount of the compound in the subject sample to a corresponding amount of the compound in one or more than one reference sample to determine the presence or absence of an increase or decrease in the amount of the compound in the subject sample;
wherein an increase or decrease in the amount of the compound in the subject sample indicates an effect caused by the anti-inflammatory drug in the subject.

52-53. (canceled)

54. The method of claim 51, wherein the compound is selected from the following group of structures:

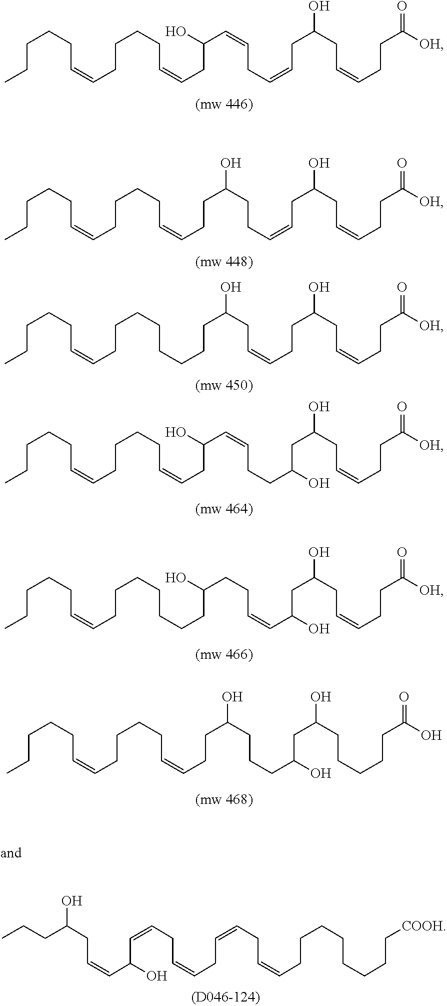

55. The method of claim 51, wherein the sample is a blood sample from said subject and is analyzed in step a) by mass spectrometry to obtain accurate mass intensity data for said compound, and the accurate mass intensity data is compared in step b) to corresponding accurate mass intensity data obtained from the one or more than one reference sample to identify an increase or decrease in accurate mass intensity.

56. The method of claim 51, wherein the sample is a blood sample from said subject and is analyzed in step a) by tandem mass spectrometry, NMR or ELISA.

57. The method of claim 51, wherein the subject treated with said anti-inflammatory drug has an inflammation and/or an inflammatory condition or disease.

58. The compound according to claim 1, labeled with a detection agent.

59. A standard comprising the compound of claim 1, or a mixture of any two or more thereof, labeled with a detection agent.

60. The standard according to claim 59, wherein the detection agent is a stable isotope or a radioisotope, an enzyme or a protein that enables detection in vitro or in vivo.

61. A kit comprising a standard according to claim 59 and instructions for quantitating an analyte or performing a diagnostic test.

62. A pharmaceutical composition comprising a pharmaceutically acceptable carrier or excipient and a compound of formula (I) as defined in claim 1.

63-64. (canceled)

65. The composition of claim 62, wherein the compound is selected from the following group of structures:

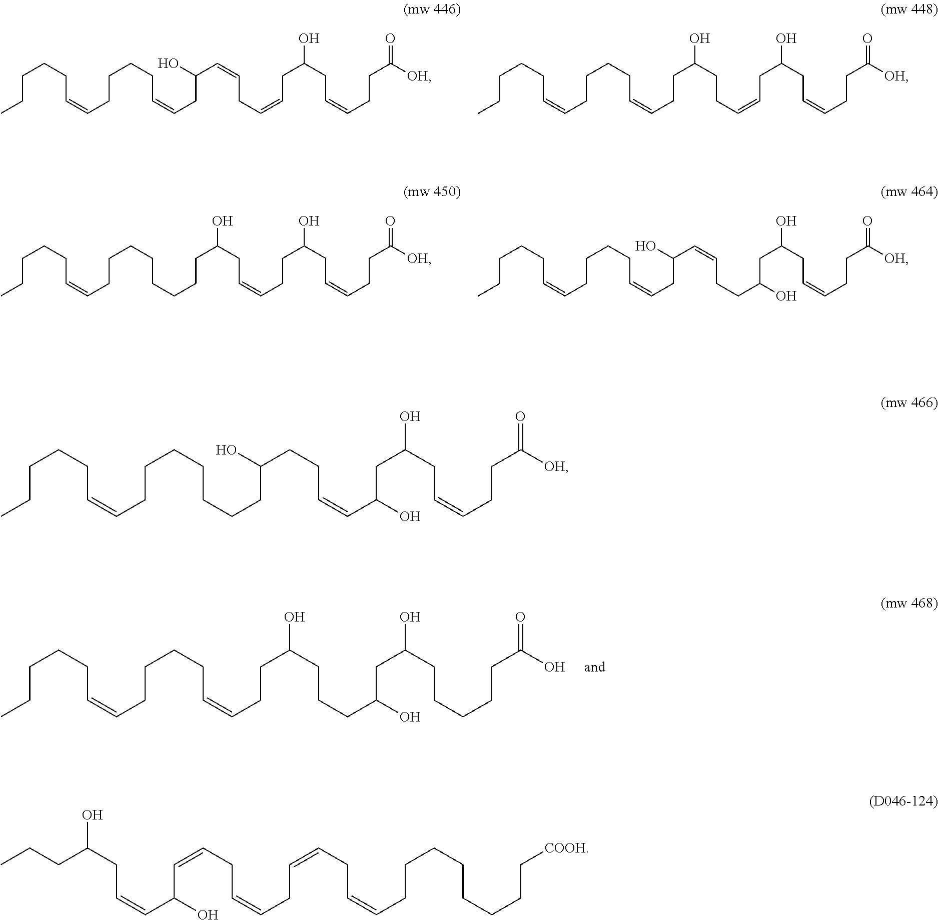

66. (canceled)

67. A combination comprising two or more compounds as defined by formula (I) as defined in claim 1.

68-69. (canceled)

70. The combination of claim 67, wherein the two or more compounds are selected from the following group of structures:

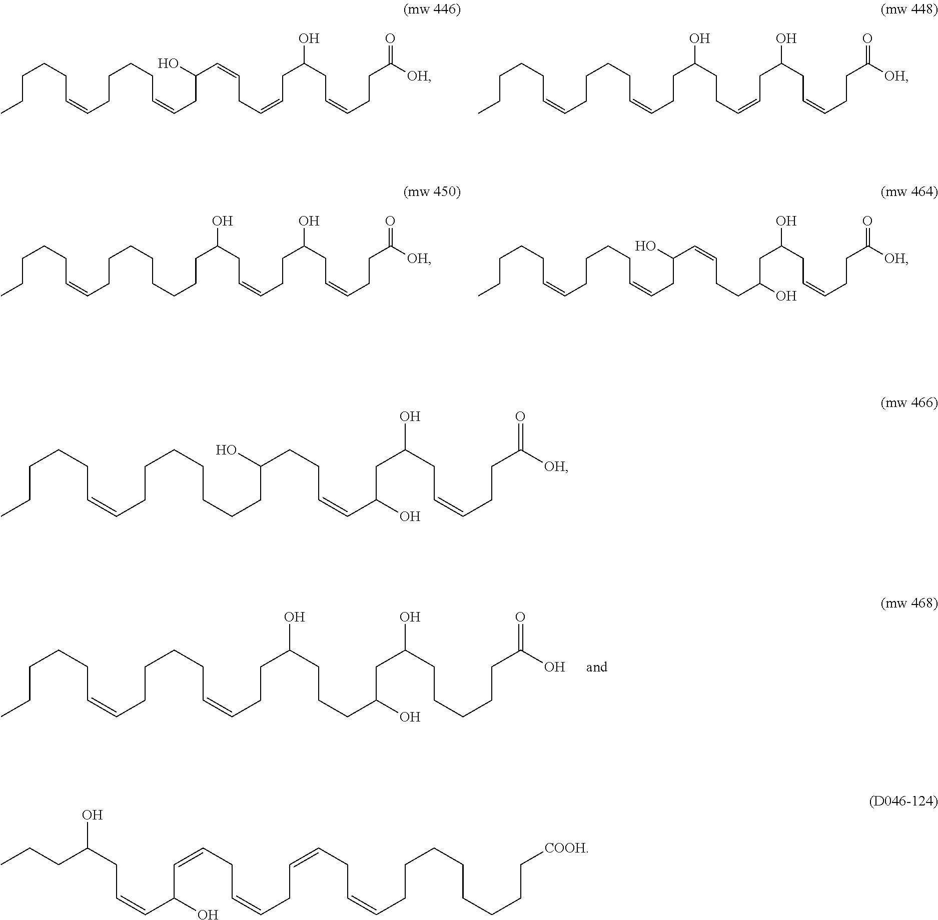

71. The combination of claim 67, which is formulated as a pharmaceutical combination, a nutritional supplement, a nutraceutical or a functional food.

72-102. (canceled)

103. A compound selected from the group consisting of:

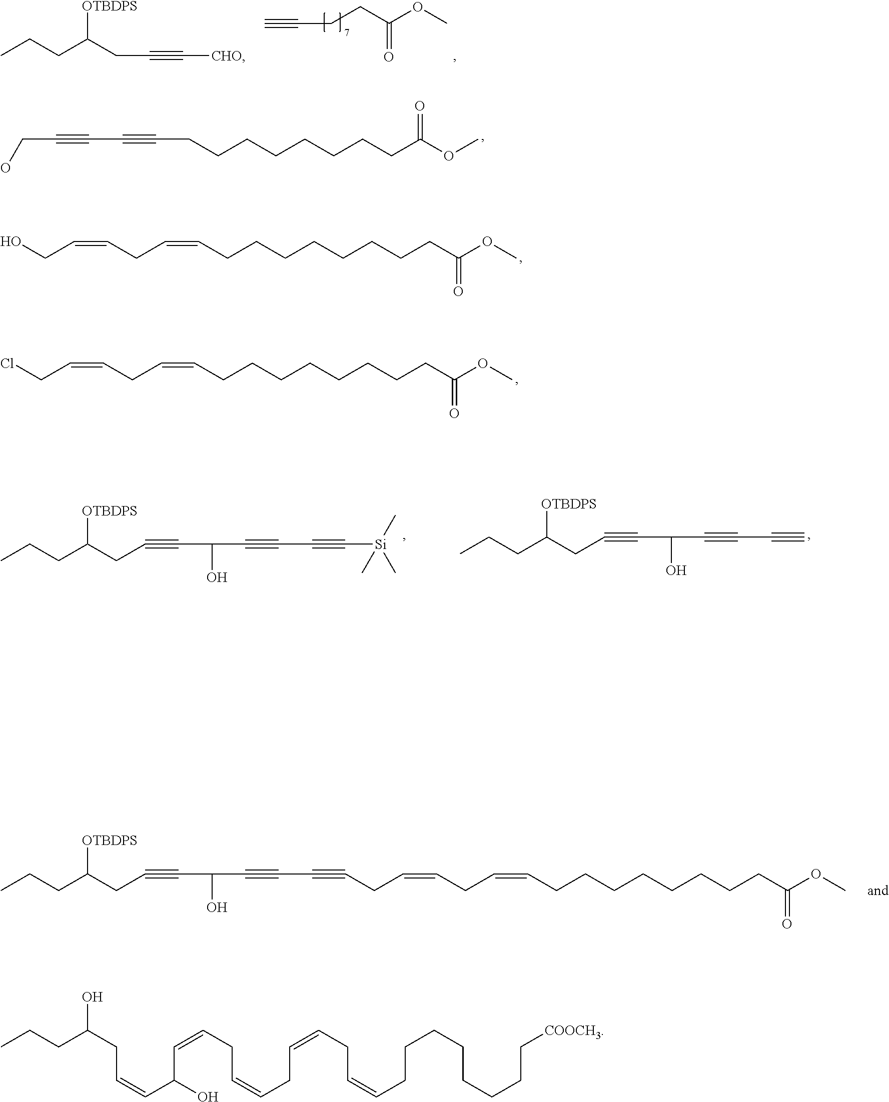

104. The compound of claim 103, wherein said compound is an intermediate in the synthesis of compound D046-124:

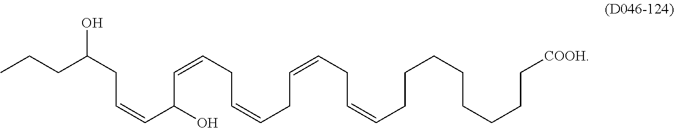

105. A method of preparing the compound D046-124 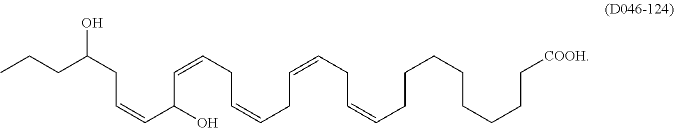
comprising:
(i) reacting a compound of formula (II): 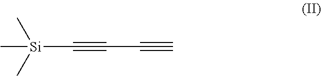
with a compound of formula (III): 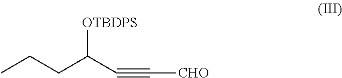
under conditions to produce a compound of formula (IV): 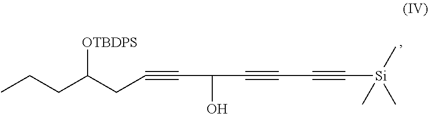
(ii) removing the TBDPS group to produce a compound of formula (V): 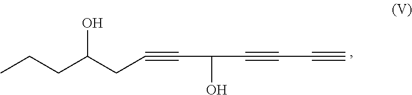
(iii) reacting the compound of formula (V) with a compound of formula (VI): 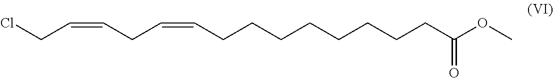
under conditions to produce a compound of formula (VII): 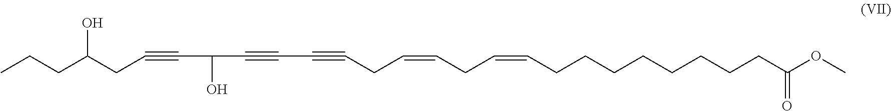
(iv) reacting the compound of formula (VII) with a catalyst under conditions to produce a compound of formula (VIII): 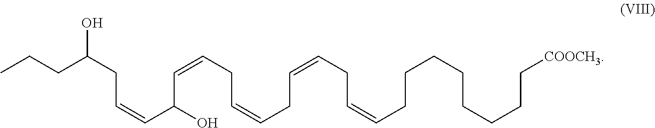
and
(v) hydrolyzing the terminal ester functional group of the compound of formula (VIII) to a carboxylic acid group thereby producing the title compound.

106. The method of claim 105, further comprising one or more purification steps to isolate the title compound.

107. The method of claim 105, wherein the compound of formula (VII) is reacted in step (iv) in the presence of a Pd catalyst with calcium carbonate under hydrogen at 1 Atm pressure to selectively convert triple bonds to double bonds and thereby produce the compound of formula (VIII).
